SEQUENCE LISTING (281 sequences)

SEQ ID NO: 1 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 2 - protein, 987 aa, Homo sapiens
MELRVLLCWASLAAALEETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKCAQLTVNLTRFPETVPRELVVPVAGSCVVDAVPAPGPSPSLYCREDGQWAEQPVTGCSCAPGFEAAEGNTKCRACAQGTFKPLSGEGSCQPCPANSHSNTIGSAVCQCRVGYFRARTDPRGAPCTTPPSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLALIAGTAVVGVVLVLVVIVVAVLCLRKQSNGREAEYSDKHGQYLIGHGTKVYIDPFTYEDPNEAVREFAKEIDVSYVKIEEVIGAGEFGEVCRGRLKAPGKKESCVAIKTLKGGYTERQRREFLSEASIMGQFEHPNIIRLEGVVTNSMPVMILTEFMENGALDSFLRLNDGQFTVIQLVGMLRGIASGMRYLAEMSYVHRDLAARNILVNSNLVCKVSDFGLSRFLEENSSDPTYTSSLGGKIPIRWTAPEAIAFRKFTSASDAWSYGIVMWEVMSFGERPYWDMSNQDVINAIEQDYRLPPPPDCPTSLHQLMLDCWQKDRNARPRFPQVVSALDKMIRNPASLKIVARENGGASHPLLDQRQPHYSAFGSVGEWLRAIKMGRYEESFAAAGFGSFELVSQISAEDLLRIGVTLAGHQKKILASVQHMKSQAKPGTPGGTGGPAPQY

SEQ ID NO: 3 - protein, 205 aa, Homo sapiens
MEFLWAPLLGLCCSLAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITHSPQAHDNPQEKRLAADDPEVRVLHSIGHSAAPRLFPLAWTVLLLPLLLLQTP

SEQ ID NO: 4 - protein, 333 aa, Homo sapiens
MAVRRDSVWKYCWGVLMVLCRTAISKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQDASSAGSTRNKDPTRRPELEAGTNGRSSTTSPFVKPNPGSSTDGNSAGHSGNNILGSEVALFAGIASGCIIFIVIIITLVVLLLKYRRRHRKHSPQHTTTLSLSTLATPKRSGNNNGSEPSDIIIPLRTADSVFCPHYEKVSGDYGHPVYIVQEMPPQSPANIYYKV

SEQ ID NO: 5 - DNA, 1849 bp, Homo sapiens
acttagaggcgcctggtcgggaagggcctggtcagctgcgtccggcggaggcagctgctgacccagctgtggactgtgccgggggcgggggacggaggggcaggagccctgggctccccgtggcgggggctgtatcatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttg

SEQ ID NO: 6 - DNA, 4369 bp, Homo sapiens
ttccagcgcagctcagcccctgcccggcccggcccgcccggctccgcgccgcagtctccctccctcccgctccgtccccgctcgggctcccaccatccccgcccgcgaggagagcactcggcccggcggcgcgagcagagccactccagggagggggggagaccgcgagcggccggctcagcccccgccacccggggcgggaccccgaggccccggagggaccccaactccagccacgtcttgctgcgcgcccgcccggcgcggccactgccagcacgctccgggcccgccgcccgcgcgcgcggcacagacgcggggccacacttggcgccgccgcccggtgccccgcacgctcgcatgggcccgcgctgagggccccgacgaggagtcccgcgcggagtatcggcgtccacccgcccagggagagtcagacctgggggggcgagggccccccaaactcagttcggatcctacccgagtgaggcggcgccatggagctccgggtgctgctctgctgggcttcgttggccgcagctttggaagagaccctgctgaacacaaaattggaaactgctgatctgaagtgggtgacattccctcaggtggacgggcagtgggaggaactgagcggcctggatgaggaacagcacagcgtgcgcacctacgaagtgtgtgacgtgcagcgtgccccgggccaggcccactggcttcgcacaggttgggtcccacggcggggcgccgtccacgtgtacgccacgctgcgcttcaccatgctcgagtgcctgtccctgcctcgggctgggcgctcctgcaaggagaccttcaccgtcttctactatgagagcgatgcggacacggccacggccctcacgccagcctggatggagaacccctacatcaaggtggacacggtggccgcggagcatctcacccggaagcgccctggggccgaggccaccgggaaggtgaatgtcaagacgctgcgtctgggaccgctcagcaaggctggcttctacctggccttccaggaccagggtgcctgcatggccctgctatccctgcacctcttctacaaaaagtgcgcccagctgactgtgaacctgactcgattcccggagactgtgcctcgggagctggttgtgcccgtggccggtagctgcgtggtggatgccgtccccgcccctggccccagccccagcctctactgccgtgaggatggccagtgggccgaacagccggtcacgggctgcagctgtgctccggggttcgaggcagctgaggggaacaccaagtgccgagcctgtgcccagggcaccttcaagcccctgtcaggagaagggtcctgccagccatgcccagccaatagccactctaacaccattggatcagccgtctgccagtgccgcgtcgggtacttccgggcacgcacagacccccggggtgcaccctgcaccacccctccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctgattgcgggcacggcagtcgtgggtgtggtcctggtcctggtggtcattgtggtcgcagttctctgcctcaggaagcagagcaatgggagagaagcagaatattcggacaaacacggacagtatctcatcggacatggtactaaggtctacatcgaccccttcacttatgaagaccctaatgaggctgtgagggaatttgcaaaagagatcgatgtctcctacgtcaagattgaagaggtgattggtgcaggtgagtttggcgaggtgtgccgggggcggctcaaggccccagggaagaaggagagctgtgtggcaatcaagaccctgaagggtggctacacggagcggcagcggcgtgagtttctgagcgaggcctccatcatgggccagttcgagcaccccaatatcatccgcctggagggcgtggtcaccaacagcatgcccgtcatgattctcacagagttcatggagaacggcgccctggactccttcctgcggctaaacgacggacagttcacagtcatccagctcgtgggcatgctgcggggcatcgcctcgggcatgcggtaccttgccgagatgagctacgtccaccgagacctggctgctcgcaacatcctagtcaacagcaacctcgtctgcaaagtgtctgactttggcctttcccgattcctggaggagaactcttccgatcccacctacacgagctccctgggaggaaagattcccatccgatggactgccccggaggccattgccttccggaagttcacttccgccagtgatgcctggagttacgggattgtgatgtgggaggtgatgtcatttggggagaggccgtactgggacatgagcaatcaggacgtgatcaatgccattgaacaggactaccggctgcccccgcccccagactgtcccacctccctccaccagctcatgctggactgttggcagaaagaccggaatgcccggccccgcttcccccaggtggtcagcgccctggacaagatgatccggaaccccgccagcctcaaaatcgtggcccgggagaatggcggggcctcacaccctctcctggaccagcggcagcctcactactcagcttttggctctgtgggcgagtggcttcgggccatcaaaatgggaagatacgaagaaagtttcgcagccgctggctttggctccttcgagctggtcagccagatctctgctgaggacctgctccgaatcggagtcactctggcgggacaccagaagaaaatcttggccagtgtccagcacatgaagtcccaggccaagccgggaaccccgggtgggacaggaggaccggccccgcagtactgacctgcaggaactccccaccccagggacaccgcctccccattttccggggcagagtggggactcacagaggcccccagccctgtgccccgctggattgcactttgagcccgtggggtgaggagttggcaatttggagagacaggatttgggggttctgccataataggaggggaaaatcaccccccagccacctcggggaactccagaccaagggtgagggcgcctttccctcaggactgggtgtgaccagaggaaaaggaagtgcccaacatctcccagcctccccaggtgcccccctcaccttgatgggtgcgttcccgcagaccaaagagagtgtgactcccttgccagctccagagtgggggggctgtcccagggggcaagaaggggtgtcagggcccagtgacaaaatcattggggtttgtagtcccaacttgctgctgtcaccaccaaactcaatcatttttttcccttgtaaatgcccctcccccagctgctgccttcatattgaaggtttttgagttttgtttttggtcttaatttttctccccgttccctttttgtttcttcgttttgtttttctaccgtccttgtcataactttgtgttggagggaacctgtttcactatggcctcctttgcccaagttgaaacaggggcccatcatcatgtctgtttccagaacagtgccttggtcatcccacatccccggaccccgcctgggacccccaagctgtgtcctatgaaggggtgtggggtgaggtagtgaaaagggcggtagttggtggtggaacccagaaacggacgccggtgcttggaggggttcttaaattatatttaaaaaagtaactttttgtataaataaaagaaaatgggacgtgtcccagctccaggggtaaaaaaaaaaaaaaaaaa

SEQ ID NO: 7 - DNA, 1590 bp, Homo sapiens
gccagatctgtgagcccagcgctgactgcgccgcggagaaagccagtgggaacccagacccataggagacccgcgtccccgctcggcctggccaggccccgcgctatggagttcctctgggcccctctcttgggtctgtgctgcagtctggccgctgctgatcgccacaccgtcttctggaacagttcaaatcccaagttccggaatgaggactacaccatacatgtgcagctgaatgactacgtggacatcatctgtccgcactatgaagatcactctgtggcagacgctgccatggagcagtacatactgtacctggtggagcatgaggagtaccagctgtgccagccccagtccaaggaccaagtccgctggcagtgcaaccggcccagtgccaagcatggcccggagaagctgtctgagaagttccagcgcttcacacctttcaccctgggcaaggagttcaaagaaggacacagctactactacatctccaaacccatccaccagcatgaagaccgctgcttgaggttgaaggtgactgtcagtggcaaaatcactcacagtcctcaggcccatgacaatccacaggagaagagacttgcagcagatgacccagaggtgcgggttctacatagcatcggtcacagtgctgccccacgcctcttcccacttgcctggactgtgctgctccttccacttctgctgctgcaaaccccgtgaaggtgtatgccacacctggccttaaagagggacaggctgaagagagggacaggcactccaaacctgtcttggggccactttcagagcccccagccctgggaaccactcccaccacaggcataagctatcacctagcagcctcaaaacgggtcagtattaaggttttcaaccggaaggaggccaaccagcccgacagtgccatccccaccttcacctcggagggatggagaaagaagtggagacagtcctttcccaccattcctgcctttaagccaaagaaacaagctgtgcaggcatggtcccttaaggcacagtgggagctgagctggaaggggccacgtggatgggcaaagcttgtcaaagatgccccctccaggagagagccaggatgcccagatgaactgactgaaggaaaagcaagaaacagtttcttgcttggaagccaggtacaggagaggcagcatgcttgggctgacccagcatctcccagcaagacctcatctgtggagctgccacagagaagtttgtagccaggtactgcattctctcccatcctggggcagcactccccagagctgtgccagcaggggggctgtgccaacctgttcttagagtgtagctgtaagggcagtgcccatgtgtacattctgcctagagtgtagcctaaagggcagggcccacgtgtatagtatctgtatataagttgctgtgtgtctgtcctgatttctacaactggagtttttttatacaatgttctttgtctcaaaataaagcaatgtgttttttcggacatgcttttctgccactccatattaaaacatatgaccattgagtccctgctaaaaaaaaaaaaaaaaaaaaaaaaaa

SEQ ID NO: 8 - DNA, 4335 bp, Homo sapiens
gcgcggagctgggagtggcttcgccatggctgtgagaagggactccgtgtggaagtactgctggggtgttttgatggttttatgcagaactgcgatttccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaagatgcaagttctgctggatcaaccaggaataaagatccaacaagacgtccagaactagaagctggtacaaatggaagaagttcgacaacaagtccctttgtaaaaccaaatccaggttctagcacagacggcaacagcgccggacattcggggaacaacatcctcggttccgaagtggccttatttgcagggattgcttcaggatgcatcatcttcatcgtcatcatcatcacgctggtggtcctcttgctgaagtaccggaggagacacaggaagcactcgccgcagcacacgaccacgctgtcgctcagcacactggccacacccaagcgcagcggcaacaacaacggctcagagcccagtgacattatcatcccgctaaggactgcggacagcgtcttctgccctcactacgagaaggtcagcggggactacgggcacccggtgtacatcgtccaggagatgcccccgcagagcccggcgaacatttactacaaggtctgagagggaccctggtggtacctgtgctttcccagaggacacctaatgtcccgatgcctcccttgagggtttgagagcccgcgtgctggagaattgactgaagcacagcaccgggggagagggacactcctcctcggaagagcccgtcgcgctggacagcttacctagtcttgtagcattcggccttggtgaacacacacgctccctggaagctggaagactgtgcagaagacgcccattcggactgctgtgccgcgtcccacgtctcctcctcgaagccatgtgctgcggtcactcaggcctctgcagaagccaagggaagacagtggtttgtggacgagagggctgtgagcatcctggcaggtgccccaggatgccacgcctggaagggccggcttctgcctggggtgcatttcccccgcagtgcataccggacttgtcacacggacctcgggctagttaaggtgtgcaaagatctctagagtttagtccttactgtctcactcgttctgttacccagggctctgcagcacctcacctgagacctccactccacatctgcatcactcatggaacactcatgtctggagtcccctcctccagccgctggcaacaacagcttcagtccatgggtaatccgttcatagaaattgtgtttgctaacaaggtgccctttagccagatgctaggctgtctgcgaagaaggctaggagttcatagaagggagtggggctggggaaagggctggctgcaattgcagctcactgctgctgcctctgaaacagaaagttggaaaggaaaaaagaaaaaagcaattaggtagcacagcactttggttttgctgagatcgaagaggccagtaggagacacgacagcacacacagtggattccagtgcatggggaggcactcgctgttatcaaatagcgatgtgcaggaagaaaagcccctcttcattccggggaacaaagacgggtattgttgggaaaggaacaggcttggagggaagggagaaagtaggccgctgatgatatattcgggcaggactgttgtggtactggcaataagatacacagctccgagctgtaggagagtcggtctgctttggatgattttttaagcagactcagctgctatacttatcacattttattaaacacagggaaagcatttaggagaatagcagagagccaaatctgacctaaaagttgaaaagccaaaggtcaaacaggctgtaattccatcatcatcgttgttattaaagaatccttatctataaaaggtaggtcagatccccctccccccaggttcctccttcccctcccgattgagccttacgacactttggtttatgcggtgctgtccgggtgccagggctgcagggtcggtactgatggaggctgcagcgcccggtgctctgtgtcaaggtgaagcacatacggcagacctcttagagtccttaagacggaagtaaattatgatgtccagggggagaaggaagataggacgtatttataataggtatatagaacacaagggatataaaatgaaagatttttactaatatatattttaaggttgcacacagtacacaccagaagatgtgaaattcatttgtggcaattaagtggtcccaatgctcagcgcttaaaaaaacaaattggacagctacttctgggaaaaacaacatcattccaaaaagaacaataatgagagcaaatgcaaaaataaccaagtcctccgaaggcatctcacggaaccgtagactaggaagtacgagccccacagagcaggaagccgatgtgactgcatcatatatttaacaatgacaagatgttccggcgtttatttctgcgttgggttttcccttgccttatgggctgaagtgttctctagaatccagcaggtcacactgggggcttcaggtgacgatttagctgtggctccctcctcctgtcctcccccgcaccccctcccttctgggaaacaagaagagtaaacaggaaacctactttttatgtgctatgcaaaatagacatctttaacatagtcctgttactatggtaacactttgctttctgaattggaagggaaaaaaaatgtagcgacagcattttaaggttctcagacctccagtgagtacctgcaaaaatgagttgtcacagaaattatgatcctctatttcctgaacctggaaatgatgttggtccaaagtgcgtgtgtgtatgtgtgagtgggtgcgtggtatacatgtgtacatatatgtataatatatatctacaatatatattatatatatctatatcatatttctgtggagggttgccatggtaaccagccacagtacatatgtaattctttccatcaccccaacctctcctttctgtgcattcatgcaagagtttcttgtaagccatcagaagttacttttaggatgggggagaggggcgagaaggggaaaaatgggaaatagtctgattttaatgaaatcaaatgtatgtatcatcagttggctacgttttggttctatgctaaactgtgaaaaatcagatgaattgataaaagagttccctgcaaccaattgaaaagtgttctgtgcgtctgttttgtgtctggtgcagaatatgacaatctaccaactgtccctttgtttgaagttggtttagctttggaaagttactgtaaatgccttgcttgtatgatcgtccctggtcacccgactttggaatttgcaccatcatgtttcagtgaagatgctgtaaataggttcagattttactgtctatggatttggggtgttacagtagccttattcacctttttaataaaaatacacatgaaaacaagaaagaaatggcttttcttacccagattgtgtacatagagcaatgttggttttttataaagtctaagcaagatgttttgtataaaatctgaattttgcaatgtatttagctacagcttgtttaacggcagtgtcattcccctttgcactgtaatgaggaaaaaatggtataaaaggttgccaaattgctgcatatttgtgccgtaattatgtaccatgaatatttatttaaaatttcgttgtccaatttgtaagtaacacagtattatgcctgagttataaatatttttttctttctttgttttattttaatagcctgtcataggttttaaatctgctttagtttcacattgcagttagccccagaaaatgaaatccgtgaagtcacattccacatctgtttcaaactgaatttgttcttaaaaaaataaaatatttttttcctatggaaaaaaaaaaaaaaaaaa

SEQ ID NO: 9 - DNA, 642 bp, Homo sapiens
agcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggacccc

SEQ ID NO: 10 - DNA, 651 bp, Homo sapiens
ccttcggctccgcggagcgtggtttcccgcctgaacggctcctccctgcacctggaatggagtgcccccctggagtctggtggccgagaggacctcacctacgccctccgctgccgggagtgccgacccggaggctcctgtgcgccctgcgggggagacctgacttttgaccccggcccccgggacctggtggagccctgggtggtggttcgagggctacgtcctgacttcacctatacctttgaggtcactgcattgaacggggtatcctccttagccacggggcccgtcccatttgagcctgtcaatgtcaccactgaccgagaggtacctcctgcagtgtctgacatccgggtgacgcggtcctcacccagcagcttgagcctggcctgggctgttccccgggcacccagtggggctgtgctggactacgaggtcaaataccatgagaagggcgccgagggtcccagcagcgtgcggttcctgaagacgtcagaaaaccgggcagagctgcgggggctgaagcggggagccagctacctggtgcaggtacgggcgcgctctgaggccggctacgggcccttcggccaggaacatcacagccagacccaactggatgagagcgagggctggcgggagcagctggccctg

SEQ ID NO: 11 - DNA, 417 bp, Homo sapiens
ctggccgctgctgatcgccacaccgtcttctggaacagttcaaatcccaagttccggaatgaggactacaccatacatgtgcagctgaatgactacgtggacatcatctgtccgcactatgaagatcactctgtggcagacgctgccatggagcagtacatactgtacctggtggagcatgaggagtaccagctgtgccagccccagtccaaggaccaagtccgctggcagtgcaaccggcccagtgccaagcatggcccggagaagctgtctgagaagttccagcgcttcacacctttcaccctgggcaaggagttcaaagaaggacacagctactactacatctccaaacccatccaccagcatgaagaccgctgcttgaggttgaaggtgactgtcagtggcaaaatcactcac

SEQ ID NO: 12 - DNA, 426 bp, Homo sapiens
tccaaatcgatagttttagagcctatctattggaattcctcgaactccaaatttctacctggacaaggactggtactatacccacagataggagacaaattggatattatttgccccaaagtggactctaaaactgttggccagtatgaatattataaagtttatatggttgataaagaccaagcagacagatgcactattaagaaggaaaatacccctctcctcaactgtgccaaaccagaccaagatatcaaattcaccatcaagtttcaagaattcagccctaacctctggggtctagaatttcagaagaacaaagattattacattatatctacatcaaatgggtctttggagggcctggataaccaggagggaggggtgtgccagacaagagccatgaagatcctcatgaaagttggacaa

SEQ ID NO: 13 - protein, 214 aa, Homo sapiens
SKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDP

SEQ ID NO: 14 - protein, 217 aa, Homo sapiens
PSAPRSVVSRLNGSSLHLEWSAPLESGGREDLTYALRCRECRPGGSCAPCGGDLTFDPGPRDLVEPWVVVRGLRPDFTYTFEVTALNGVSSLATGPVPFEPVNVTTDREVPPAVSDIRVTRSSPSSLSLAWAVPRAPSGAVLDYEVKYHEKGAEGPSSVRFLKTSENRAELRGLKRGASYLVQVRARSEAGYGPFGQEHHSQTQLDESEGWREQLAL

SEQ ID NO: 15 - protein, 139 aa, Homo sapiens
LAAADRHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQHEDRCLRLKVTVSGKITH

SEQ ID NO: 16 - protein, 142 aa, Homo sapiens
SKSIVLEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTSNGSLEGLDNQEGGVCQTRAMKILMKVGQ

SEQ ID NO: 17 - protein, 138 aa, Homo sapiens
APPRLICDSRVLERYLLEAKEAENITRVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 18 - protein, 147 aa, Homo sapiens
APPRLICDSRVLERYLLEAKEAENITTGCAEHCSLNENITVPDTKVNFYAWKRMEVGQQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 19 - protein, 137 aa, Homo sapiens
APPRLICDSRVLERYLLEAKEAENITTGCAEHCSLNENITVPDTKVNFYALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 20 - protein, 135 aa, Homo sapiens
APPRLICDSRVLERYLLEAKEAENITTGCAEHCSLNENITVPDTKVNFYAWKRMEPWEPLQLHVDKAVSGLRSLTTLLRALGAQKEAISPPDAASAAPLRTITADTFRKLFRVYSNFLRGKLKLYTGEACRTGDR

SEQ ID NO: 21 - protein, 60 aa, Homo sapiens
APPRLICDSRVLERYLLEAKEAENITTGCAEHCSLNENITVPPGVGQLFPAVGAPAAACG

SEQ ID NO: 22 - protein, 41 aa, Homo sapiens
APPRLICDSRVLERYLLEAKEAENITTGCAEHCSLNENNHC

SEQ ID NO: 23 - protein, 26 aa, Homo sapiens
APPRLICDSRVLERYLLEAKEAENIT

SEQ ID NO: 24 - protein, 26 aa, Homo sapiens
APPRLICDSRVLEAYLLEAKEAENIT

SEQ ID NO: 25 - protein, 26 aa, Homo sapiens
APPRLICDSRVLEEYLLEAKEAENIT

SEQ ID NO: 26 - protein, 16 aa, Homo sapiens
APPRLICDSRVLERYL

SEQ ID NO: 27 - protein, 6 aa, Homo sapiens
APPRLI

SEQ ID NO: 28 - protein, 106 aa, Homo sapiens
APPRLICDSRVLERYILEAKEAENVTMGCAEGPRLSENITVPDTKVNFYAWKRMEKELMSPPDTTPPAPLRTLTVDTFCKLFRVYANFLRGKLKLYTGEVCRRGDR

SEQ ID NO: 29 - protein, 153 aa, Homo sapiens
APPRLICEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 30 - protein, 162 aa, Homo sapiens
APPRLICDSRVLERYLLEAKEAENITTGCAEHCSLNENITVPDFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 31 - protein, 158 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDLTTLLRALGAQKEAISPPDAASAAPLRTITADTFRKLFRVYSNFLRGKLKLYTGEACRTGDR

SEQ ID NO: 32 - protein, 158 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFGKLKLYTGEACRTGDR

SEQ ID NO: 33 - protein, 160 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEA

SEQ ID NO: 34 - protein, 159 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGE

SEQ ID NO: 35 - protein, 158 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTG

SEQ ID NO: 36 - protein, 157 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYT

SEQ ID NO: 37 - protein, 156 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLY

SEQ ID NO: 38 - protein, 155 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKL

SEQ ID NO: 39 - protein, 154 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLK

SEQ ID NO: 40 - protein, 153 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKL

SEQ ID NO: 41 - protein, 152 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGK

SEQ ID NO: 42 - protein, 151 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRG

SEQ ID NO: 43 - protein, 150 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLR

SEQ ID NO: 44 - protein, 149 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFL

SEQ ID NO: 45 - protein, 148 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNF

SEQ ID NO: 46 - protein, 147 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSN

SEQ ID NO: 47 - protein, 146 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYS

SEQ ID NO: 48 - protein, 145 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVY

SEQ ID NO: 49 - protein, 144 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRV

SEQ ID NO: 50 - protein, 143 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFR

SEQ ID NO: 51 - protein, 142 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLF

SEQ ID NO: 52 - protein, 141 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKL

SEQ ID NO: 53 - protein, 140 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRK

SEQ ID NO: 54 - protein, 139 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFR

SEQ ID NO: 55 - protein, 138 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTF

SEQ ID NO: 56 - protein, 137 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADT

SEQ ID NO: 57 - protein, 136 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITAD

SEQ ID NO: 58 - protein, 135 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITA

SEQ ID NO: 59 - protein, 134 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTIT

SEQ ID NO: 60 - protein, 133 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTI

SEQ ID NO: 61 - protein, 132 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRT

SEQ ID NO: 62 - protein, 131 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLR

SEQ ID NO: 63 - protein, 130 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPL

SEQ ID NO: 64 - protein, 129 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAP

SEQ ID NO: 65 - protein, 128 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAA

SEQ ID NO: 66 - protein, 127 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASA

SEQ ID NO: 67 - protein, 126 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAAS

SEQ ID NO: 68 - protein, 125 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAA

SEQ ID NO: 69 - protein, 124 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDA

SEQ ID NO: 70 - protein, 123 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPD

SEQ ID NO: 71 - protein, 122 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPP

SEQ ID NO: 72 - protein, 121 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISP

SEQ ID NO: 73 - protein, 120 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAIS

SEQ ID NO: 74 - protein, 119 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAI

SEQ ID NO: 75 - protein, 118 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEA

SEQ ID NO: 76 - protein, 117 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKE

SEQ ID NO: 77 - protein, 116 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQK

SEQ ID NO: 78 - protein, 115 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQ

SEQ ID NO: 79 - protein, 114 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGA

SEQ ID NO: 80 - protein, 113 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALG

SEQ ID NO: 81 - protein, 112 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRAL

SEQ ID NO: 82 - protein, 111 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRA

SEQ ID NO: 83 - protein, 110 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLR

SEQ ID NO: 84 - protein, 109 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLL

SEQ ID NO: 85 - protein, 108 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTL

SEQ ID NO: 86 - protein, 107 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTT

SEQ ID NO: 87 - protein, 106 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLT

SEQ ID NO: 88 - protein, 105 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSL

SEQ ID NO: 89 - protein, 104 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRS

SEQ ID NO: 90 - protein, 103 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLR

SEQ ID NO: 91 - protein, 102 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGL

SEQ ID NO: 92 - protein, 101 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSG

SEQ ID NO: 93 - protein, 100 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVS

SEQ ID NO: 94 - protein, 99 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAV

SEQ ID NO: 95 - protein, 98 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKA

SEQ ID NO: 96 - protein, 97 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDK

SEQ ID NO: 97 - protein, 96 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVD

SEQ ID NO: 98 - protein, 95 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHV

SEQ ID NO: 99 - protein, 94 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLH

SEQ ID NO: 100 - protein, 93 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQL

SEQ ID NO: 101 - protein, 92 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQ

SEQ ID NO: 102 - protein, 91 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPL

SEQ ID NO: 103 - protein, 90 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEP

SEQ ID NO: 104 - protein, 89 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWE

SEQ ID NO: 105 - protein, 88 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPW

SEQ ID NO: 106 - protein, 87 aa, Homo sapiens
APPRLICDSRVLERYLLEAKEAENITTGCAEHCSLNENITVPDTKVNFYAWKRMEVGQQAVEVWQGLALLSEAVLRGQALLVNSSQP

SEQ ID NO: 107 - protein, 86 aa, Homo sapiens
APPRLICDSRVLERYLLEAKEAENITTGCAEHCSLNENITVPDTKVNFYAWKRMEVGQQAVEVWQGLALLSEAVLRGQALLVNSSQ

SEQ ID NO: 108 - protein, 85 aa, Homo sapiens
APPRLICDSRVLERYLLEAKEAENITTGCAEHCSLNENITVPDTKVNFYAWKRMEVGQQAVEVWQGLALLSEAVLRGQALLVNSS

SEQ ID NO: 109 - protein, 84 aa, Homo sapiens
APPRLICDSRVLERYLLEAKEAENITTGCAEHCSLNENITVPDTKVNFYAWKRMEVGQQAVEVWQGLALLSEAVLRGQALLVNS

SEQ ID NO: 110 - protein, 83 aa, Homo sapiens
APPRLICDSRVLERYLLEAKEAENITTGCAEHCSLNENITVPDTKVNFYAWKRMEVGQQAVEVWQGLALLSEAVLRGQALLVN

SEQ ID NO: 111 - protein, 82 aa, Homo sapiens
APPRLICDSRVLERYLLEAKEAENITTGCAEHCSLNENITVPDTKVNFYAWKRMEVGQQAVEVWQGLALLSEAVLRGQALLV

SEQ ID NO: 112 - protein, 81 aa, Homo sapiens
APPRLICDSRVLERYLLEAKEAENITTGCAEHCSLNENITVPDTKVNFYAWKRMEVGQQAVEVWQGLALLSEAVLRGQALL

SEQ ID NO: 113 - protein, 80 aa, Homo sapiens
APPRLICDSRVLERYLLEAKEAENITTGCAEHCSLNENITVPDTKVNFYAWKRMEVGQQAVEVWQGLALLSEAVLRGQAL

SEQ ID NO: 114 - protein, 79 aa, Homo sapiens
APPRLICDSRVLERYLLEAKEAENITTGCAEHCSLNENITVPDTKVNFYAWKRMEVGQQAVEVWQGLALLSEAVLRGQA

SEQ ID NO: 115 - protein, 78 aa, Homo sapiens
APPRLICDSRVLERYLLEAKEAENITTGCAEHCSLNENITVPDTKVNFYAWKRMEVGQQAVEVWQGLALLSEAVLRGQ

SEQ ID NO: 116 - protein, 77 aa, Homo sapiens
APPRLICDSRVLERYLLEAKEAENITTGCAEHCSLNENITVPDTKVNFYAWKRMEVGQQAVEVWQGLALLSEAVLRG

SEQ ID NO: 117 - protein, 76 aa, Homo sapiens
APPRLICDSRVLERYLLEAKEAENITTGCAEHCSLNENITVPDTKVNFYAWKRMEVGQQAVEVWQGLALLSEAVLR

SEQ ID NO: 118 - protein, 75 aa, Homo sapiens
APPRLICDSRVLERYLLEAKEAENITTGCAEHCSLNENITVPDTKVNFYAWKRMEVGQQAVEVWQGLALLSEAVL

SEQ ID NO: 119 - protein, 74 aa, Homo sapiens
APPRLICDSRVLERYLLEAKEAENITTGCAEHCSLNENITVPDTKVNFYAWKRMEVGQQAVEVWQGLALLSEAV

SEQ ID NO: 120 - protein, 73 aa, Homo sapiens
APPRLICDSRVLERYLLEAKEAENITTGCAEHCSLNENITVPDTKVNFYAWKRMEVGQQAVEVWQGLALLSEA

SEQ ID NO: 121 - protein, 72 aa, Homo sapiens
APPRLICDSRVLERYLLEAKEAENITTGCAEHCSLNENITVPDTKVNFYAWKRMEVGQQAVEVWQGLALLSE

SEQ ID NO: 122 - protein, 71 aa, Homo sapiens
APPRLICDSRVLERYLLEAKEAENITTGCAEHCSLNENITVPDTKVNFYAWKRMEVGQQAVEVWQGLALLS

SEQ ID NO: 123 - protein, 70 aa, Homo sapiens
APPRLICDSRVLERYLLEAKEAENITTGCAEHCSLNENITVPDTKVNFYAWKRMEVGQQAVEVWQGLALL

SEQ ID NO: 124 - protein, 69 aa, Homo sapiens
APPRLICDSRVLERYLLEAKEAENITTGCAEHCSLNENITVPDTKVNFYAWKRMEVGQQAVEVWQGLAL

SEQ ID NO: 125 - protein, 68 aa, Homo sapiens
APPRLICDSRVLERYLLEAKEAENITTGCAEHCSLNENITVPDTKVNFYAWKRMEVGQQAVEVWQGLA

SEQ ID NO: 126 - protein, 67 aa, Homo sapiens
APPRLICDSRVLERYLLEAKEAENITTGCAEHCSLNENITVPDTKVNFYAWKRMEVGQQAVEVWQGL

SEQ ID NO: 127 - protein, 66 aa, Homo sapiens
APPRLICDSRVLERYLLEAKEAENITTGCAEHCSLNENITVPDTKVNFYAWKRMEVGQQAVEVWQG

SEQ ID NO: 128 - protein, 65 aa, Homo sapiens
APPRLICDSRVLERYLLEAKEAENITTGCAEHCSLNENITVPDTKVNFYAWKRMEVGQQAVEVWQ

SEQ ID NO: 129 - protein, 64 aa, Homo sapiens
APPRLICDSRVLERYLLEAKEAENITTGCAEHCSLNENITVPDTKVNFYAWKRMEVGQQAVEVW

SEQ ID NO: 130 - protein, 63 aa, Homo sapiens
APPRLICDSRVLERYLLEAKEAENITTGCAEHCSLNENITVPDTKVNFYAWKRMEVGQQAVEV

SEQ ID NO: 131 - protein, 62 aa, Homo sapiens
APPRLICDSRVLERYLLEAKEAENITTGCAEHCSLNENITVPDTKVNFYAWKRMEVGQQAVE

SEQ ID NO: 132 - protein, 61 aa, Homo sapiens
APPRLICDSRVLERYLLEAKEAENITTGCAEHCSLNENITVPDTKVNFYAWKRMEVGQQAV

SEQ ID NO: 133 - protein, 60 aa, Homo sapiens
APPRLICDSRVLERYLLEAKEAENITTGCAEHCSLNENITVPDTKVNFYAWKRMEVGQQA

SEQ ID NO: 134 - protein, 59 aa, Homo sapiens
APPRLICDSRVLERYLLEAKEAENITTGCAEHCSLNENITVPDTKVNFYAWKRMEVGQQ

SEQ ID NO: 135 - protein, 58 aa, Homo sapiens
APPRLICDSRVLERYLLEAKEAENITTGCAEHCSLNENITVPDTKVNFYAWKRMEVGQ

SEQ ID NO: 136 - protein, 57 aa, Homo sapiens
APPRLICDSRVLERYLLEAKEAENITTGCAEHCSLNENITVPDTKVNFYAWKRMEVG

SEQ ID NO: 137 - protein, 56 aa, Homo sapiens
APPRLICDSRVLERYLLEAKEAENITTGCAEHCSLNENITVPDTKVNFYAWKRMEV

SEQ ID NO: 138 - protein, 55 aa, Homo sapiens
APPRLICDSRVLERYLLEAKEAENITTGCAEHCSLNENITVPDTKVNFYAWKRME

SEQ ID NO: 139 - protein, 54 aa, Homo sapiens
APPRLICDSRVLERYLLEAKEAENITTGCAEHCSLNENITVPDTKVNFYAWKRM

SEQ ID NO: 140 - protein, 53 aa, Homo sapiens
APPRLICDSRVLERYLLEAKEAENITTGCAEHCSLNENITVPDTKVNFYAWKR

SEQ ID NO: 141 - protein, 52 aa, Homo sapiens
APPRLICDSRVLERYLLEAKEAENITTGCAEHCSLNENITVPDTKVNFYAWK

SEQ ID NO: 142 - protein, 51 aa, Homo sapiens
APPRLICDSRVLERYLLEAKEAENITTGCAEHCSLNENITVPDTKVNFYAW

SEQ ID NO: 143 - protein, 50 aa, Homo sapiens
APPRLICDSRVLERYLLEAKEAENITTGCAEHCSLNENITVPDTKVNFYA

SEQ ID NO: 144 - protein, 49 aa, Homo sapiens
APPRLICDSRVLERYLLEAKEAENITTGCAEHCSLNENITVPDTKVNFY

SEQ ID NO: 145 - protein, 48 aa, Homo sapiens
APPRLICDSRVLERYLLEAKEAENITTGCAEHCSLNENITVPDTKVNF

SEQ ID NO: 146 - protein, 47 aa, Homo sapiens
APPRLICDSRVLERYLLEAKEAENITTGCAEHCSLNENITVPDTKVN

SEQ ID NO: 147 - protein, 46 aa, Homo sapiens
APPRLICDSRVLERYLLEAKEAENITTGCAEHCSLNENITVPDTKV

SEQ ID NO: 148 - protein, 45 aa, Homo sapiens
APPRLICDSRVLERYLLEAKEAENITTGCAEHCSLNENITVPDTK

SEQ ID NO: 149 - protein, 44 aa, Homo sapiens
APPRLICDSRVLERYLLEAKEAENITTGCAEHCSLNENITVPDT

SEQ ID NO: 150 - protein, 43 aa, Homo sapiens
APPRLICDSRVLERYLLEAKEAENITTGCAEHCSLNENITVPD

SEQ ID NO: 151 - protein, 42 aa, Homo sapiens
APPRLICDSRVLERYLLEAKEAENITTGCAEHCSLNENITVP

SEQ ID NO: 152 - protein, 41 aa, Homo sapiens
APPRLICDSRVLERYLLEAKEAENITTGCAEHCSLNENITV

SEQ ID NO: 153 - protein, 40 aa, Homo sapiens
APPRLICDSRVLERYLLEAKEAENITTGCAEHCSLNENIT

SEQ ID NO: 154 - protein, 39 aa, Homo sapiens
APPRLICDSRVLERYLLEAKEAENITTGCAEHCSLNENI

SEQ ID NO: 155 - protein, 38 aa, Homo sapiens
APPRLICDSRVLERYLLEAKEAENITTGCAEHCSLNEN

SEQ ID NO: 156 - protein, 37 aa, Homo sapiens
APPRLICDSRVLERYLLEAKEAENITTGCAEHCSLNE

SEQ ID NO: 157 - protein, 36 aa, Homo sapiens
APPRLICDSRVLERYLLEAKEAENITTGCAEHCSLN

SEQ ID NO: 158 - protein, 35 aa, Homo sapiens
APPRLICDSRVLERYLLEAKEAENITTGCAEHCSL

SEQ ID NO: 159 - protein, 34 aa, Homo sapiens
APPRLICDSRVLERYLLEAKEAENITTGCAEHCS

SEQ ID NO: 160 - protein, 33 aa, Homo sapiens
APPRLICDSRVLERYLLEAKEAENITTGCAEHC

SEQ ID NO: 161 - protein, 32 aa, Homo sapiens
APPRLICDSRVLERYLLEAKEAENITTGCAEH

SEQ ID NO: 162 - protein, 31 aa, Homo sapiens
APPRLICDSRVLERYLLEAKEAENITTGCAE

SEQ ID NO: 163 - protein, 30 aa, Homo sapiens
APPRLICDSRVLERYLLEAKEAENITTGCA

SEQ ID NO: 164 - protein, 29 aa, Homo sapiens
APPRLICDSRVLERYLLEAKEAENITTGC

SEQ ID NO: 165 - protein, 28 aa, Homo sapiens
APPRLICDSRVLERYLLEAKEAENITTG

SEQ ID NO: 166 - protein, 27 aa, Homo sapiens
APPRLICDSRVLERYLLEAKEAENITT

SEQ ID NO: 167 - protein, 26 aa, Homo sapiens
APPRLICDSRVLERYLLEAKEAENIT

SEQ ID NO: 168 - protein, 25 aa, Homo sapiens
APPRLICDSRVLERYLLEAKEAENI

SEQ ID NO: 169 - protein, 24 aa, Homo sapiens
APPRLICDSRVLERYLLEAKEAEN

SEQ ID NO: 170 - protein, 23 aa, Homo sapiens
APPRLICDSRVLERYLLEAKEAE

SEQ ID NO: 171 - protein, 22 aa, Homo sapiens
APPRLICDSRVLERYLLEAKEA

SEQ ID NO: 172 - protein, 21 aa, Homo sapiens
APPRLICDSRVLERYLLEAKE

SEQ ID NO: 173 - protein, 20 aa, Homo sapiens
APPRLICDSRVLERYLLEAK

SEQ ID NO: 174 - protein, 19 aa, Homo sapiens
APPRLICDSRVLERYLLEA

SEQ ID NO: 175 - protein, 18 aa, Homo sapiens
APPRLICDSRVLERYLLE

SEQ ID NO: 176 - protein, 17 aa, Homo sapiens
APPRLICDSRVLERYLL

SEQ ID NO: 177 - protein, 16 aa, Homo sapiens
APPRLICDSRVLERYL

SEQ ID NO: 178 - protein, 15 aa, Homo sapiens
APPRLICDSRVLERY

SEQ ID NO: 179 - protein, 14 aa, Homo sapiens
APPRLICDSRVLER

SEQ ID NO: 180 - protein, 13 aa, Homo sapiens
APPRLICDSRVLE

SEQ ID NO: 181 - protein, 12 aa, Homo sapiens
APPRLICDSRVL

SEQ ID NO: 182 - protein, 11 aa, Homo sapiens
APPRLICDSRV

SEQ ID NO: 183 - protein, 10 aa, Homo sapiens
APPRLICDSR

SEQ ID NO: 184 - protein, 9 aa, Homo sapiens
APPRLICDS

SEQ ID NO: 185 - protein, 8 aa, Homo sapiens
APPRLICD

SEQ ID NO: 186 - protein, 7 aa, Homo sapiens
APPRLIC

SEQ ID NO: 187 - protein, 166 aa, Homo sapiens
APPRLICASRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 188 - protein, 166 aa, Homo sapiens
APPRLICRSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 189 - protein, 166 aa, Homo sapiens
APPRLICDSRVLEAYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 190 - protein, 166 aa, Homo sapiens
APPRLICDSRVLEEYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 191 - protein, 166 aa, Homo sapiens
APPRLICDSRVLERYLLEAAEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 192 - protein, 166 aa, Homo sapiens
APPRLICDSRVLERYLLEAEEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 193 - protein, 166 aa, Homo sapiens
APPRLICDSRVLERYLLEAKEAENITTGCAEHCSLNENITVPDAKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 194 - protein, 166 aa, Homo sapiens
APPRLICDSRVLERYLLEAKEAENITTGCAEHCSLNENITVPDTAVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 195 - protein, 166 aa, Homo sapiens
APPRLICDSRVLERYLLEAKEAENITTGCAEHCSLNENITVPDTEVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 196 - protein, 166 aa, Homo sapiens
APPRLICDSRVLERYLLEAKEAENITTGCAEHCSLNENITVPDTKANFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 197 - protein, 166 aa, Homo sapiens
APPRLICDSRVLERYLLEAKEAENITTGCAEHCSLNENITVPDTKVAFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 198 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDAAVSGLRSLTTLLRALGAQKEAISPPDAASAAPLRTITADTFRKLFRVYSNFLRGKLKLYTGEACRTGDR

SEQ ID NO: 199 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDEAVSGLRSLTTLLRALGAQKEAISPPDAASAAPLRTITADTFRKLFRVYSNFLRGKLKLYTGEACRTGDR

SEQ ID NO: 200 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVAGLRSLTTLLRALGAQKEAISPPDAASAAPLRTITADTFRKLFRVYSNFLRGKLKLYTGEACRTGDR

SEQ ID NO: 201 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLASLTTLLRALGAQKEAISPPDAASAAPLRTITADTFRKLFRVYSNFLRGKLKLYTGEACRTGDR

SEQ ID NO: 202 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLESLTTLLRALGAQKEAISPPDAASAAPLRTITADTFRKLFRVYSNFLRGKLKLYTGEACRTGDR

SEQ ID NO: 203 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRALTTLLRALGAQKEAISPPDAASAAPLRTITADTFRKLFRVYSNFLRGKLKLYTGEACRTGDR

SEQ ID NO: 204 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFAVYSNFLRGKLKLYTGEACRTGDR

SEQ ID NO: 205 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFEVYSNFLRGKLKLYTGEACRTGDR

SEQ ID NO: 206 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSAFLRGKLKLYTGEACRTGDR

SEQ ID NO: 207 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLAGKLKLYTGEACRTGDR

SEQ ID NO: 208 - protein, 166 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLEGKLKLYTGEACRTGDR

SEQ ID NO: 209 - protein, 27 aa, Homo sapiens
APPRLICDSRVLERYLLEAKEAENITT

SEQ ID NO: 210 - protein, 21 aa, Homo sapiens
NENITVPDTKVNFYAWKRMEV

SEQ ID NO: 211 - protein, 27 aa, Homo sapiens
NSSQPWEPLQLHVDKAVSGLRSLTTLL

SEQ ID NO: 212 - protein, 18 aa, Homo sapiens
FRKLFRVYSNFLRGKLKL

SEQ ID NO: 213 - protein, 193 aa, Homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 214 - protein, 181 aa, Homo sapiens
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALTPAWMENPYIKVDTVAAEHLTRKRPGAEATGKVNVKTLRLGPLSKAGFYLAFQDQGACMALLSLHLFYKKC

SEQ ID NO: 215 - protein, 199 aa, Homo sapiens
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASALTPAWMENPYIKVDTVAAEHLTRKRPGAEATGKVNVKTLRLGPLSKAGFYLAFQDQGACMALLSLHLFYKKC

SEQ ID NO: 216 - protein, 206 aa, Homo sapiens
EETLLNTKLETADLKWVTFPQVDGQWEELSGLDEEQHSVRTYEVCDVQRAPGQAHWLRTGWVPRRGAVHVYATLRFTMLECLSLPRAGRSCKETFTVFYYESDADTATALSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 217 - DNA, 20 bp, Homo sapiens
atggaggcctcgctcagaaa

SEQ ID NO: 218 - DNA, 20 bp, Homo sapiens
tacctgaaggtcaggcgaac

SEQ ID NO: 219 - RNA, 21 nt, Homo sapiens
ggugaaugucaagacgcuguu

SEQ ID NO: 220 - RNA, 21 nt, Homo sapiens
cagcgucuugacauucaccuu

SEQ ID NO: 221 - RNA, 21 nt, Homo sapiens
aguuaauaucaagacgcuguu

SEQ ID NO: 222 - RNA, 21 nt, Homo sapiens
cagcgucuugauauuaacuuu

SEQ ID NO: 223 - DNA, 21 bp, Artificial Sequence
  Synthetic oligonucleotide
cgcugacccugaaguucatuu

SEQ ID NO: 224 - RNA, 21 nt, Artificial Sequence
  Description of Artificial Sequence Synthetic oligonucleotide
augaacuucagggucagcguu

SEQ ID NO: 225 - DNA, 16 bp, Homo sapiens
cgcggagatgggggtg

SEQ ID NO: 226 - DNA, 17 bp, Homo sapiens
acagatgaccaggtgtg

SEQ ID NO: 227 - DNA, 10 bp, Homo sapiens
ttccagcgca

SEQ ID NO: 228 - DNA, 15 bp, Homo sapiens
ggcgccatggagctc

SEQ ID NO: 229 - DNA, 16 bp, Homo sapiens
gcagtactgacctgca

SEQ ID NO: 230 - DNA, 14 bp, Homo sapiens
ccagctccaggggt

SEQ ID NO: 231 - DNA, 15 bp, Homo sapiens
tgtatcatggaccac

SEQ ID NO: 232 - DNA, 16 bp, Homo sapiens
ttgctcttaggacacc

SEQ ID NO: 233 - DNA, 14 bp, Homo sapiens
tgtatagttttttt

SEQ ID NO: 234 - protein, 107 aa, Homo sapiens
LEPVSWSSLNPKFLSGKGLVIYPKIGDKLDIICPRAEAGRPYEYYKLYLVRPEQAAACSTVLDPNVLVTCNRPEQEIRFTIKFQEFSPNYMGLEFKKHHDYYITSTS

SEQ ID NO: 235 - protein, 109 aa, Homo sapiens
LEPIYWNSSNSKFLPGQGLVLYPQIGDKLDIICPKVDSKTVGQYEYYKVYMVDKDQADRCTIKKENTPLLNCAKPDQDIKFTIKFQEFSPNLWGLEFQKNKDYYIISTS

SEQ ID NO: 236 - protein, 119 aa, Homo sapiens
VADRYAVYWNSSNPRFQRGDYHIDVCINDYLDVFCPHYEDSVPEDKTERYVLYMVNFDGYSACDHTSKGFKRWECNRPHSPNGPLKFSEKFQLFTPFSLGFEFRPGREYFYISSAIPDN

SEQ ID NO: 237 - protein, 123 aa, Homo sapiens
RYAVYWNRSNPRFHAGAGDDGGGYTVEVSINDYLDIYCPHYGAPLPPAERMEHYVLYMVNGEGHASCDHRQRGFKRWECNRPAAPGGPLKFSEKFQLFTPFSLGFEFRPGHEYYYISATPPNA

SEQ ID NO: 238 - protein, 114 aa, Homo sapiens
RHVVYWNSSNPRLLRGDAVVELGLNDYLDIVCPHYEGPGPPEGPETFALYMVDWPGYESCQAEGPRAYKRWVCSLPFGHVQFSEKIQRFTPFSLGFEFLPGETYYYISVPTPES

SEQ ID NO: 239 - protein, 117 aa, Homo sapiens
RHTVFWNSSNPKFRNEDYTIHVQLNDYVDIICPHYEDHSVADAAMEQYILYLVEHEEYQLCQPQSKDQVRWQCNRPSAKHGPEKLSEKFQRFTPFTLGKEFKEGHSYYYISKPIHQH

SEQ ID NO: 240 - protein, 105 aa, Homo sapiens
DPKFESKAALLAARGPEELLCFTERLEDLVCFWEEAASAGVGPGNYSFSYQLEDEPWKLCRLHQAPTARFWCSLPTADTSSFVPLELRVTAASGAPRYHRVIHIN

SEQ ID NO: 241 - protein, 166 aa, Artificial Sequence
  Description of Artificial Sequence Synthetic consensus sequence
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 242 - RNA, 21 nt, Homo sapiens
caauagccacucuaacaccuu

SEQ ID NO: 243 - RNA, 21 nt, Homo sapiens
gguguuagaguggcuauuguu

SEQ ID NO: 244 - RNA, 21 nt, Homo sapiens
ggggcccgucccauuugaguu

SEQ ID NO: 245 - RNA, 21 nt, Homo sapiens
cucaaaugggacgggccccuu

SEQ ID NO: 246 - RNA, 21 nt, Homo sapiens
cugaucugaagugggugacuu

SEQ ID NO: 247 - RNA, 21 nt, Homo sapiens
gucacccacuucagaucaguu

SEQ ID NO: 248 - RNA, 21 nt, Homo sapiens
aagacccuaaugaggcuguuu

SEQ ID NO: 249 - RNA, 21 nt, Homo sapiens
acagccucauuagggucuuuu

SEQ ID NO: 250 - RNA, 21 nt, Homo sapiens
ucgaugucuccuacgucaauu

SEQ ID NO: 251 - RNA, 21 nt, Homo sapiens
uugacguaggagacaucgauu

SEQ ID NO: 252 - RNA, 21 nt, Homo sapiens
auugaagaggugauugguguu

SEQ ID NO: 253 - RNA, 21 nt, Homo sapiens
caccaaucaccucuucaauuu

SEQ ID NO: 254 - RNA, 21 nt, Homo sapiens
ggaguuacgggauugugauuu

SEQ ID NO: 255 - RNA, 21 nt, Homo sapiens
aucacaaucccguaacuccuu

SEQ ID NO: 256 - RNA, 21 nt, Homo sapiens
gguacuaaggucuacaucguu

SEQ ID NO: 257 - RNA, 21 nt, Homo sapiens
cgauguagaccuuaguaccuu

SEQ ID NO: 258 - RNA, 21 nt, Homo sapiens
guccugacuucaccuauacuu

SEQ ID NO: 259 - RNA, 21 nt, Homo sapiens
guauaggugaagucaggacuu

SEQ ID NO: 260 - RNA, 21 nt, Homo sapiens
ugccgcgucggguacuuccuu

SEQ ID NO: 261 - RNA, 21 nt, Homo sapiens
ggaaguacccgacgcggcauu

SEQ ID NO: 262 - RNA, 19 nt, Homo sapiens
ccgaagagcuucugugcuu

SEQ ID NO: 263 - RNA, 19 nt, Homo sapiens
aagcacagaagcucuucgg

SEQ ID NO: 264 - RNA, 20 nt, Artificial Sequence
  Description of Artificial Sequence Synthetic oligonucleotide
uucuccgaacguugucacgu

SEQ ID NO: 265 - RNA, 19 nt, Artificial Sequence
  Description of Artificial Sequence Synthetic oligonucleotide
acgugacacguucggagaa

SEQ ID NO: 266 - RNA, 19 nt, Homo sapiens
cagccaauagccacucuaa

SEQ ID NO: 267 - RNA, 19 nt, Homo sapiens
uuagaguggcuauuggcug

SEQ ID NO: 268 - protein, 4 aa, Homo sapiens
MGVH

SEQ ID NO: 269 - protein, 6 aa, Homo sapiens
CRTGDR

SEQ ID NO: 270 - protein, 5 aa, Homo sapiens
MELRV

SEQ ID NO: 271 - protein, 7 aa, Homo sapiens
GGPAPQY

SEQ ID NO: 272 - protein, 5 aa, Homo sapiens
MDHLG

SEQ ID NO: 273 - protein, 5 aa, Homo sapiens
YVACS

SEQ ID NO: 274 - RNA, 19 nt, Homo sapiens
gaucugaagugggugacau

SEQ ID NO: 275 - RNA, 19 nt, Homo sapiens
augucacccacuucagauc

SEQ ID NO: 276 - RNA, 19 nt, Homo sapiens
cccauuugagccugucaau

SEQ ID NO: 277 - RNA, 19 nt, Homo sapiens
auugacaggcucaaauggg

SEQ ID NO: 278 - RNA, 19 nt, Homo sapiens
gaucugaagugggugacau

SEQ ID NO: 279 - RNA, 19 nt, Homo sapiens
augucacccacuucagauc

SEQ ID NO: 280 - DNA, 58 bp, Homo sapiens
ccggtgccagctttgagtacactatctcgagatagtgtactcaaagctggcatttttg

SEQ ID NO: 281 - DNA, 57 bp, Homo sapiens
ccggtgatctgaagtgggtgacattctcgagaaygycacccacttcagatcattttt

(19) United States
(12) Patent Application Publication
(10) Pub. No.: US 2013/0071854 A1
(43) Pub. Date: Mar. 21, 2013

(54) METHODS FOR THE IDENTIFICATION AND CHARACTERIZATION OF HDAC INTERACTING COMPOUNDS

(71) Applicants: Gerard Drewes, Heidelberg (DE); Marcus Bantscheff, Dielheim (DE); Ulrich Kruse, Dossenheim (DE); Carsten Hopf, Mannheim (DE); Paola Grandi, Heidelberg (DE)

(72) Inventors: Gerard Drewes, Heidelberg (DE); Marcus Bantscheff, Dielheim (DE); Ulrich Kruse, Dossenheim (DE); Carsten Hopf, Mannheim (DE); Paola Grandi, Heidelberg (DE)

(21) Appl. No.: 13/389,716

(22) PCT Filed: Aug. 13, 2010
(86) PCT No.: PCT/EP2010/005000

§ 371 Date: Aug. 22, 2012

Related U.S. Application Data
(60) Provisional application No. 61/357,820, filed on Jun. 23, 2010.

(30) Foreign Application Priority Data
Aug. 14, 2009 (EP) 09010522.2

(51) Int. Cl. G01N 33/573

(52) U.S. Cl. CPC G01N 33/573
USPC 435/7.4

(57) ABSTRACT

The present invention relates to methods for the identification and characterization (e.g. selectivity profiling) of HDAC interacting compounds using protein preparations derived from cells endogenously expressing HDACs or cell preparations containing said HDACs.

The present invention relates to methods for the identification and characterization (e.g. selectivity profiling) of HDAC interacting compounds.

The approval of the drug vorinostat (Zolinza, Merck) by the FDA for the treatment of cutaneous T-cell lymphoma in October 2006 significantly increased the interest in developing inhibitors for the class of enzymes known as histone deacetylases (HDACs). The discovery of the HDAC inhibitor vorinostat (suberoylanilide hydroxamic acid; SAHA) resulted from efforts to improve first-generation hybrid polar compounds—originally derived from DMSO—that acted as differentiation inducers of transformed cells. Subsequently, it was discovered that SAHA potently inhibits several HDACs, for example HDAC1, HDAC2, HDAC3 and HDAC6. Thus, SAHA represents a non-selective HDAC inhibitor and is sometimes referred to as a “pan-HDAC” inhibitor. The mechanisms underlying SAHA's anticancer activity are complex and not fully characterized. For example, SAHA induces the accumulation of acetylated histones with an effect on gene expression. In addition, non-histone proteins are substrates for HDACs and the effects on these proteins could contribute to its anticancer activities (Marks and Breslow, 2007. Nature Biotechnology 25(1):84-90; Grant et al., 2007. Nature Reviews Drug Discovery 6, 21-22).

HDACs catalyse the removal of acetyl groups from lysine residues in histone amino termini, leading to chromatin condensation and changes in gene expression. In humans 18 HDACs have been identified and subdivided into four classes based on their homology to yeast HDACs, their subcellular localization and their enzymatic activities. The class I HDACs (1, 2, 3 and 8) are homologous to the yeast RPD3 protein, can generally be detected in the nucleus and show ubiquitous expression in human cell lines and tissues. Class II HDACs (4, 5, 6, 7, 9 and 10) are similar to the yeast Hda proteins and can shuttle between the nucleus and the cytoplasm. HDAC6 can deacetylate the cytoskeletal protein α-tubulin. The class III HDACs (SIRT1, 2, 3, 4, 5, 6 and 7) are homologues of the yeast protein Sir2 and require NAD⁺ for their activity. HDAC11 is the sole member of class IV HDACs (Bolden et al., 2006. Nat. Rev. Drug Discov. 5(9):769-784).

Histone acetylation is a dynamic process controlled by HDACs and histone acetyltransferases (HATs) and the balance between these two processes together with other protein and DNA modifications such as DNA methylation regulates chromatin accessibility and gene expression. These reversible heritable changes in gene function occur without a change in the sequence of nuclear DNA and are therefore referred to as epigenetic gene regulation. HDACs play a key role in developmental processes, normal physiology and disease states. The ubiquitous expression and high homology between class I HDACs suggests functional redundancy among the HDACs in vivo. However, deletion of each member of the class I HDAC family leads to lethality, indicating a unique role of each HDAC in development (Haberland et al., 2009. Nat. Rev. Genet. 10(1):32-42).

Typically HDACs are present within multisubunit protein complexes together with other components that determine HDAC target gene specificity due to interactions with sequence-specific DNA-binding proteins (Cunliffe 2008. Curr. Opin. Genet. Dev. 18(5):404-410; Yang and Seto, 2008. Nat. Rev. Mol. Cell. Biol. 9(3):206-218). Class I HDACs are found primarily in four distinct multiprotein complexes known as the Sin3, NuRD, CoREST and NCoR/SMRT complexes.

Mammalians have two Sin3 homologues, Sin3A and Sin3B. The Sin3A complex contains as components HDAC 1, HDAC2, RbAp46 (retinoblastoma (RB) associated protein-46) and RbAp48. In addition, the complex contains RBP1 (Rb-binding protein-1), SAP180 (which is similar to RBP1), SDS3, BRMS1 (breast cancer metastasis suppressor-1, similar to SDS3), SAP30,SAP18 and ING1 (or ING2). Because some of the subunits have different isoforms, there are several distinct Sin3Acomplexes. The Sin3B complex shares some similarity to the Sin3Acomplex but might also contain distinct subunits (Yang and Seto, 2008. Nat. Rev. Mol. Cell. Biol. 9(3):206-218).

The nucleosome remodeling and deacetylase (NuRD) complex contains a catalytic core of HDAC1, HDAC2, RbAp46 and RbAp48. In addition, this complex contains MTA (metastasis-associated) proteins, the nucleosome-remodelling ATPase Mi-2 (dermatomyositis-specific autoantigen), MBD2 (methyl CpG-binding domain-2) and p66. Through MBD2, this complex is recruited for DNA methylation-dependent gene silencing.

Three mammalian MTA isoforms (MTA1, -2 and -3) have been identified that contain SANT domains, which are crucial for the integrity and deacetylase activity of NuRD. Mi-2 and p66 have α- and β-isoforms, enabling the formation of distinct complexes.

The CoREST complex contains the corepressor protein CoREST interacting with the transcriptional repressor REST, HDAC1 and HDAC2. This complex is also referred to as neuronal corepressor complex because the REST zinc finger protein to target sites in the promoters of neuronal genes. One subunit encodes the Lys-specific demethylase-1 (LSD1) protein which functions as a demethylase for K4-methylated histone H3.

The NCoR/SNRT complex contains the SANT-domain-containing co-repressors SMRT, NCoR and SMRTER and is targeted to DNA by transcription factor such as CSL and nuclear receptors.

Several HDAC inhibitors are in preclinical development and clinical trials for the treatment of a wide variety of diseases including cancer, inflammatory, cardiac, and neurodegenerative diseases (Bolden et al., 2006. Nat. Rev. Drug Discov. 5(9):769-784; Haberland et al., 2009. Nat. Rev. Genet. 10(1):32-42). It is expected that the development of selective HDAC inhibitors targeting only one member of the HDAC family should lead to improved efficacy and drug safety compared to non-selective “pan-HDAC inhibitors” (Kalin et al., 2009. Curr. Opin. Chem. Biol. 13:1-9; Balasubramanian et al., 2009. Cancer Lett. 280(2):211-21).

The majority of in vitro studies of HDAC activity are performed using in vitro biochemical assays. These assays are also used for the identification of HDAC inhibitors (Hauser et al., 2009. Curr. Top. Med. Chem. 9(3):227-234). For example, HDAC activity can be measured using purified recombinant enzyme in solution-based assays with acetylated peptide substrates (Blackwell et al., 2008. Life Sciences 82(21-22):1050-1058).

Typically, these assays require the availability of purified or recombinant HDACs. However, not all HDACs can be produced will sufficient enzymatic activity to allow for inhibitor screening (Blackwell et al., 2008. Life Sciences 82(21-22):1050-1058). In addition, some preparations of HDACs expressed in insect cells are contaminated with endogenous insect HDACs making the interpretation of assay results ambiguous.

Another, although not in all instances necessary prerequisite for the identification of selective HDAC inhibitors is a method that allows to determine the target selectivity of these molecules. For example, it can be intended to provide molecules that bind to and inhibit a particular drug target but do not interact with a closely related target, inhibition of which could lead to unwanted side effects. Conventionally, large panels of individual enzyme assays are used to assess the inhibitory effect of a compound for HDACs (Khan et al., 2008. Biochem. J. 409(2):581-9; Blackwell et al., 2008. Life Sci. 82(21-22):1050-1058).

More recently, chemical proteomics methods have been described using acitivity-based probes that irreversibly bind to HDACs (Salisbury and Cravatt, 2007. PNAS 104(4):1171-1176; Salisbury and Cravatt, 2008. J. Am. Chem. Soc. 130(7):2184-2194).

In view of the above, there is a need for providing effective tools and methods for the identification and selectivity profiling of HDAC interacting compounds.

In a first aspect, the present invention relates to a method for the identification of a histone deacetylase (HDAC) interacting compound, comprising the steps of
a) providing an HDAC protein complex containing protein preparation derived from a cell endogenously expressing an HDAC,
b) contacting the protein preparation with a non-proteinaceous HDAC ligand immobilized on a solid support and with a given compound thereby allowing the reversible binding of said ligand to said HDAC protein complex, and
c) determining to what extent in step b) a binding between the ligand and the protein complex has occurred.

In a second aspect, the present invention relates to a method for the identification of a histone deacetylase (HDAC) interacting compound, comprising the steps of
a) providing two aliquots of an HDAC protein complex containing protein preparation derived from a cell endogenously expressing an HDAC,
b) contacting one aliquot with a non-proteinaceous HDAC ligand immobilized on a solid support thereby allowing the reversible binding of said ligand to said HDAC protein complex,
c) contacting the other aliquot with a non-proteinaceous HDAC ligand immobilized on a solid support and with a given compound thereby allowing the reversible binding of said ligand to said HDAC protein complex, and
d) determining to what extent in steps b) and c) a binding between the ligand and the protein complex has occurred.

In a third aspect, the present invention relates to a method for the identification of a histone deacetylase (HDAC) interacting compound, comprising the steps of:
a) providing two aliquots of a cell preparation each comprising at least one cell endogenously expressing an HDAC,
b) incubating one aliquot with a given compound,
c) harvesting the cells of each aliquot,
d) lysing the cells of each aliquot in order to obtain protein preparations containing an HDAC protein complex,
e) contacting the protein preparations with a non-proteinaceous HDAC ligand immobilized on a solid support thereby allowing the reversible binding of said ligand to said HDAC protein complex, and
f) determining to what extent in step e) a binding between the ligand and the protein complex has occurred.

In a fourth aspect, the present invention relates to a method for the identification of a histone deacetylase (HDAC) interacting compound, comprising the steps of
a) providing at least two aliquots of an HDAC containing protein preparation derived from a cell endogenously expressing an HDAC,
b) contacting one aliquot with a non-proteinaceous HDAC ligand immobilized on a solid support thereby allowing the reversible binding of said ligand to HDAC,
c) contacting the remaining aliquots with an HDAC ligand immobilized on a solid support and with various concentrations of a compound thereby allowing the reversible binding of said ligand to HDAC,
d) separating the HDAC from the ligand, and
e) determining by mass spectrometry or immunodetection the amount of HDAC eluted from the ligand, thereby determining to what extent in steps b) and c) a binding between the ligand and the protein has occurred.

In the context of the present invention, it has been found that the methods of the present invention are suitable for the identification of compounds interacting with HDACs. The methods according to the first three aspects of the invention are especially suitable because they rely on the use of HDAC complexes which should ensure that the HDAC is present as much as possible in its natural environment. The method according to the fourth aspect of the invention has been found especially suitable in that the binding characteristics of known HDAC inhibitors could be very well characterized using this method of the invention. Especially, as shown in Examples 6 and 8 with the help of said method of the invention, it is possible to determine the IC₅₀ values of known HDAC interacting compounds yielding different selectivity profiles compared to selectivity profiles based on methods known in the art. Furthermore, Examples 2 to 5 (see also Tables 6 to 9 and FIGS. 27 to 29) demonstrate that, with the help of said method of the invention, it is possible to detect a high variety of components of HDAC-containing protein complexes.

According to the present invention, the expression “HDAC” or “histone deacetylase” means enzymes that remove acetyl groups from histones or other substrate proteins. These enzymes are known in the art.

Examples of HDACs are:

### Class I HDACs (HDAC1, HDAC2, HDAC3, HDAC8);

class IIa HDAC(HDAC4, HDAC5, HDAC7, HDAC9);
class IIb HDAC(HDAC6, HDAC10);
class IV HDAC(HDAC11).

Each of them can be used in the context of the present invention. Preferred examples are HDAC1, HDAC2, HDAC3, HDAC6, HDAC8 and HDAC10.

According to the present invention, the expression “HDAC” relates to both human and other proteins of this family. The expression especially includes functionally active derivatives thereof, or functionally active fragments thereof, or a homologues thereof, or variants encoded by a nucleic acid that hybridizes to the nucleic acid encoding said protein under low stringency conditions. Preferably, these low stringency conditions include hybridization in a buffer comprising 35% formamide, 5×SSC, 50 mM Tris-HCl (pH 7.5), 5 mM EDTA, 0.02% PVP, 0.02% BSA, 100 μg/ml denatured salmon sperm DNA, and 10% (wt/vol) dextran sulfate for 18-20 hours at 40° C., washing in a buffer consisting of 2×SSC, 25 mM Tris-HCl (pH 7.4), 5 mM EDTA, and 0.1% SDS for 1-5 hours at 55° C., and washing in a buffer consisting of 2×SSC, 25 mM Tris-HCl (pH 7.4) 5 mM EDTA, and 0.1% SDS for 1.5 hours at 60° C.

Moreover, according to the present invention, the expression “HDAC” includes mutant forms said HDACs.

In the aspects of the invention, first a protein preparation containing said HDAC (see the fourth aspect of the invention) or HDAC complex (see the first three aspects of the invention) is provided.

The methods of the present invention can be performed with any protein preparation as a starting material, as long as the respective HDAC or HDAC complex is present in the preparation and is derived from a cell endogenously expressing an HDAC. Examples include a liquid mixture of several proteins, a cell lysate, a partial cell lysate which contains not all proteins present in the original cell or a combination of several cell lysates. The term “protein preparation” also includes dissolved purified protein.

In the context of the present invention, the term “endogenously” means that the respective cell expresses said HDAC without being transfected with an HDAC-encoding nucleic acid. This ensures that the HDAC is present, as much as possible, in its natural environment, especially is contained in a naturally occurring HDAC protein complex as discussed above.

In another aspect of the invention, aliquots of a cell preparation are provided as the starting material. In the context of the present invention, the term “cell preparation” refers to any preparation containing at least one cell with the desired properties. Suitable cell preparations are described below.

The presence of the HDACs in a protein preparation of interest can be detected on Western blots probed with antibodies that are specifically directed against said HDAC. Alternatively, also mass spectrometry (MS) could be used to detect the HDACs (see below).

The presence of a given HDAC complex can be e.g. determined by determining whether, in a given protein preparation, HDAC is complexed to another known component of said complex. Corresponding methods are well known in the art and include co-immunoprecipitation or purification of HDAC under conditions allowing that the HDAC complex is not dissociated.

Cell lysates or partial cell lysates can be obtained by isolating cell organelles (e.g. nucleus, mitochondria, ribosomes, golgi etc.) first and then preparing protein preparations derived from these organelles. Methods for the isolation of cell organelles are known in the art (Chapter 4.2 Purification of Organelles from Mammalian Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

In addition, protein preparations can be prepared by fractionation of cell extracts thereby enriching specific types of proteins such as cytoplasmic or membrane proteins (Chapter 4.3 Subcellular Fractionation of Tissue Culture Cells in “Current Protocols in Protein Science”, Editors: John. E. Coligan, Ben M. Dunn, Hidde L. Ploegh, David W. Speicher, Paul T. Wingfield; Wiley, ISBN: 0-471-14098-8).

Furthermore protein preparations from body fluids can be used (e.g. blood, cerebrospinal fluid, peritoneal fluid and urine).

For example whole embryo lysates derived from defined development stages or adult stages of model organisms such as C. elegans can be used. In addition, whole organs such as heart dissected from mice can be the source of protein preparations. These organs can also be perfused in vitro in order to obtain a protein preparation.

In a preferred embodiment of the methods of the invention, the provision of a protein preparation includes the steps of harvesting at least one cell containing the HDAC or the HDAC protein complex and lysing the cell.

Suitable cells for this purpose as well as for the cell preparations used as the starting material in one aspect of the present invention are those cells or tissues where the HDACs are endogenously expressed. In any given cell or tissue only a subset of the HDACs may be expressed. Therefore it may be necessary to generate multiple protein preparations from a variety of cell types and tissues to cover the HDAC family, especially for selectivity profiling of HDAC inhibitors. As established cell lines may not reflect the physiological expression pattern of HDACs, primary animal or human cells may be used, for example cells isolated from blood samples.

Therefore, in a preferred embodiment, cells isolated from peripheral blood represent a suitable biological material. Procedures for the preparation and culture of human lymphocytes and lymphocyte subpopulations obtained from peripheral blood (PBLs) are widely known (W. E Biddison, Chapter 2.2 “Preparation and culture of human lymphocytes” in Current Protocols in Cell Biology, 1998, John Wiley & Sons, Inc.). For example, density gradient centrifugation is a method for the separation of lymphocytes from other blood cell populations (e.g. erythrocytes and granulocytes). Human lymphocyte subpopulations can be isolated via their specific cell surface receptors which can be recognized by monoclonal antibodies. The physical separation method involves coupling of these antibody reagents to magnetic beads which allow the enrichment of cells that are bound by these antibodies (positive selection).

As an alternative to primary human cells cultured cell lines (e.g. MOLT-4 cells, Jurkat, Ramos, HeLa or K-562 cells) can be used.

In a preferred embodiment, the cell is part of a cell culture system and methods for the harvest of a cell out of a cell culture system are known in the art (literature supra).

The choice of the cell will mainly depend on the expression of the HDACs, since it has to be ensured that the protein is principally present in the cell of choice. In order to determine whether a given cell is a suitable starting system for the methods of the invention, methods like Westernblot, PCR-based nucleic acids detection methods, Northernblots and DNA-microarray methods (“DNA chips”) might be suitable in order to determine whether a given protein of interest is present in the cell.

The choice of the cell may also be influenced by the purpose of the study. If the in vivo efficacy for a given drug needs to be analyzed then cells or tissues may be selected in which the desired therapeutic effect occurs (e.g. B-cells). By contrast, for the elucidation of protein targets mediating unwanted side effects the cell or tissue may be analysed in which the side effect is observed (e.g. cardiomyocytes, vascular smooth muscle or epithelium cells).

Furthermore, it is envisaged within the present invention that the cell containing the HDACs or the HDAC complex may be obtained from an organism, e.g. by biopsy. Corresponding methods are known in the art. For example, a biopsy is a diagnostic procedure used to obtain a small amount of tissue, which can then be examined microscopically or with biochemical methods. Biopsies are important to diagnose, classify and stage a disease, but also to evaluate and monitor drug treatment.

It is encompassed within the present invention that by the harvest of the at least one cell, the lysis is performed simultaneously. However, it is equally preferred that the cell is first harvested and then separately lysed.

Methods for the lysis of cells are known in the art (Karwa and Mitra: Sample preparation for the extraction, isolation, and purification of Nuclei Acids; chapter 8 in “Sample Preparation Techniques in Analytical Chemistry”, Wiley 2003, Editor: Somenath Mitra, print ISBN: 0471328456; online ISBN: 0471457817). Lysis of different cell types and tissues can be achieved by homogenizers (e.g. Potter-homogenizer), ultrasonic desintegrators, enzymatic lysis, detergents (e.g. NP-40, Triton X-100, CHAPS, SDS), osmotic shock, repeated freezing and thawing, or a combination of these methods.

According to the methods of the invention, the protein preparation containing the HDAC or the HDAC protein complex is contacted with an HDAC ligand thereby allowing the reversible binding of the HDAC complex or the HDAC to the ligand.

In the context of the present invention, the term “HDAC ligand” denotes every molecule being able to bind to HDAC. Preferably, the HDAC ligand is a small molecule as defined below.

In the present invention, the term “allowing the reversible binding” means that reversibly a complex is formed between the HDAC complex or the HDAC and the ligand. Conditions allowing the reversible binding of molecules to proteins or protein complexes are known in the art. The skilled person will know which conditions can be applied in order to enable the formation of said complex.

In the context of the present invention, compounds are identified which interfere with the binding between the HDAC ligand and an HDAC or an HDAC protein complex present in a cell or protein preparation.

According to the aspects of the invention as defined above, the HDAC ligand is a non-proteinaceous ligand and is immobilized on a solid support. However, it is also included within the present invention that in another aspect, a proteinaceous ligand is used which may or may not be immobilized on a solid support.

Throughout the invention, the term “solid support” relates to every undissolved support being able to immobilize a small molecule ligand on its surface. The solid support may be selected from the group consisting of agarose, modified agarose, sepharose beads (e.g. NHS-activated sepharose), latex, cellulose, and ferro- or ferrimagnetic particles.

Methods and strategies for choosing appropriate solid supports and for coupling compounds to said solid supports are known in the art (see e.g. Wong, Shan S. Chemistry of protein conjugation and cross-linking (1991), CRC Press, Inc. ISBN 0-8493-5886-8 Chapter 12: Conjugation of proteins to solid matrices, pages 295-318).

The HDAC ligand may be coupled to the solid support either covalently or non-covalently. Non-covalent binding includes binding via biotin affinity ligands binding to steptavidin matrices.

Preferably, the HDAC ligand is covalently coupled to the solid support.

Methods for immobilizing compounds on solid supports are known in the art. In general, before the coupling, the matrixes can contain active groups such as NHS, Carbodimide etc. to enable the coupling reaction with the immobilization compound. The ligand can be coupled to the solid support by direct coupling (e.g. using functional groups such as amino-, sulfhydryl-, carboxyl-, hydroxyl-, aldehyde-, and ketone groups) and by indirect coupling (e.g. via biotin, biotin being covalently attached to the ligand and non-covalent binding of biotin to streptavidin which is bound directly to the solid support). The linkage to the solid support material may involve cleavable and non-cleavable linkers. The cleavage may be achieved by enzymatic cleavage or treatment with suitable chemical methods.

Consequently, in case that the HDAC ligand is immobilized on a solid support, the term “allowing the reversible binding” includes all conditions under which such binding is possible. This includes the possibility of having the solid support on an immobilized phase and pouring the lysate onto it. In another preferred embodiment, it is also included that the solid support is in a particulate form and mixed with the cell lysate. Such conditions are known to the person skilled in the art.

In the context of non-covalent binding, the binding between the HDAC ligand and the HDAC or the HDAC complex is, e.g., via salt bridges, hydrogen bonds, hydrophobic interactions or a combination thereof.

In a preferred embodiment, the steps of the formation of said complex are performed under essentially physiological conditions. The physical state of proteins within cells is described in Petty, 1998 (Howard R. Petty, Chapter 1, Unit 1.5 in: Juan S. Bonifacino, Mary Dasso, Joe B. Harford, Jennifer Lippincott-Schwartz, and Kenneth M. Yamada (eds.) Current Protocols in Cell Biology Copyright© 2003 John Wiley & Sons, Inc. All rights reserved. DOI: 10.1002/0471143030.cb0101s00Online Posting Date: May, 2001Print Publication Date: October, 1998).

The contacting under essentially physiological conditions has the advantage that the interactions between the ligand, the cell preparation (i.e. the HDAC to be characterized) and optionally the compound reflect as much as possible the natural conditions. “Essentially physiological conditions” are inter alia those conditions which are present in the original, unprocessed sample material. They include the physiological protein concentration, pH, salt concentration, buffer capacity and post-translational modifications of the proteins involved. The term “essentially physiological conditions” does not require conditions identical to those in the original living organism, wherefrom the sample is derived, but essentially cell-like conditions or conditions close to cellular conditions. The person skilled in the art will, of course, realize that certain constraints may arise due to the experimental set-up which will eventually lead to less cell-like conditions. For example, the eventually necessary disruption of cell walls or cell membranes when taking and processing a sample from a living organism may require conditions which are not identical to the physiological conditions found in the organism. Suitable variations of physiological conditions for practicing the methods of the invention will be apparent to those skilled in the art and are encompassed by the term “essentially physiological conditions” as used herein. In summary, it is to be understood that the term “essentially physiological conditions” relates to conditions close to physiological conditions, as e.g. found in natural cells, but does not necessarily require that these conditions are identical.

For example, “essentially physiological conditions” may comprise 50-200 mM NaCl or KCl, pH 6.5-8.5, 20-37° C., and 0.001-10 mM divalent cation (e.g. Mg++, Ca++,); more preferably about 150 m NaCl or KCl, p17.2 to 7.6, 5 mM divalent cation and often include 0.01-1.0 percent non-specific protein (e.g. BSA). A non-ionic detergent (Tween, NP-40, Triton-X100) can often be present, usually at about 0.001 to 2%, typically 0.05-0.2% (volume/volume). For general guidance, the following buffered aqueous conditions may be applicable: 10-250 mM NaCl, 5-50 mM Tris HCl, pH5-8, with optional addition of divalent cation(s) and/or metal chelators and/or non-ionic detergents.

Preferably, “essentially physiological conditions” mean a pH of from 6.5 to 7.5, preferably from 7.0 to 7.5, and/or a buffer concentration of from 10 to 50 mM, preferably from 25 to 50 mM, and/or a concentration of monovalent salts (e.g. Na or K) of from 120 to 170 mM, preferably 150 mM. Divalent salts (e.g. Mg or Ca) may further be present at a concentration of from 1 to 5 mM, preferably 1 to 2 mM, wherein more preferably the buffer is selected from the group consisting of Tris-HCl or HEPES.

According to the second aspect of the invention, in steps b) and c) aliquots are contacted with an HDAC ligand. In a preferred embodiment of the invention, the ligand used in steps b) and c) is the same.

The skilled person will appreciate that between the individual steps of the methods of the invention, washing steps may be necessary. Such washing is part of the knowledge of the person skilled in the art. The washing serves to remove non-bound components of the cell lysate from the solid support. Nonspecific (e.g. simple ionic) binding interactions can be minimized by adding low levels of detergent or by moderate adjustments to salt concentrations in the wash buffer.

According to the methods of the invention, the read-out system is whether a binding between the HDAC complex and the HDAC ligand (first three aspects of the invention) or whether a binding HDAC and the HDAC ligand (fourth aspect of the invention) has occurred.

Methods for determining whether a binding between a ligand and a protein or protein complex has occurred are known in the art. These methods include in situ methods where the binding is assessed without separating the protein or protein complex from the ligand.

For example, anti-HDAC antibodies can be used in combination with the ALPHAScreen technology can be used where the excitation of a donor bead at 680 nm produces singlet oxygen which can diffuse to an acceptor bead undergoing a chemiluminescent reaction (Glickman et al., 2002. J. Biomol. Screen. 7(1):3-10).

In a preferred embodiment of the first three aspects according to the invention as well as according to the fourth aspect of the invention, the binding between the ligand and the HDAC complex or protein is determined by separating bound HDAC or HDAC complex from the ligand and subsequent determination of the HDAC or the HDAC complex. This subsequent determination of the HDAC or HDAC complex may either be the detection of the HDAC or the HDAC complex in the eluate or the determination of their amount.

In general, the fact that, in the methods of the invention, a binding between the HDAC ligand and the HDAC complex or the HDAC as occurred preferably indicates that the compound does not completely inhibit the binding. On the other hand, if no binding takes place in the presence of the compound, the compound is presumably a strong interactor with the HDAC, which is indicative for its therapeutic potential. In case that the amount is determined, the less HDAC or HDAC complex can be detected in the eluate, the stronger the respective compound interacts with the HDAC, which is indicative for its therapeutic potential.

Consequently, in a preferred embodiment of the methods of the invention, a reduced binding observed for the aliquot incubated with the compound in comparison to the aliquot not incubated with the compound indicates that HDAC is a target of the compound

According to invention, separating means every action which destroys the interactions between the ligand and the HDAC or the HDAC protein complex. This includes in a preferred embodiment the elution of the HDAC or the HDAC protein complex from the ligand. The elution can be achieved by using non-specific reagents (ionic strength, pH value, detergents).

Such non-specific methods for destroying the interaction are principally known in the art and depend on the nature of the ligand enzyme interaction. Principally, change of ionic strength, the pH value, the temperature or incubation with detergents are suitable methods to dissociate the target enzymes from the immobilized compound. The application of an elution buffer can dissociate binding partners by extremes of pH value (high or low pH; e.g. lowering pH by using 0.1 M citrate, pH2-3), change of ionic strength (e.g. high salt concentration using NaI, KI, MgCl₂, or KCl), polarity reducing agents which disrupt hydrophobic interactions (e.g. dioxane or ethylene glycol), or denaturing agents (chaotropic salts or detergents such as Sodium-docedyl-sulfate, SDS; Review: Subramanian A., 2002, Immunoaffinty chromatography).

In some cases, the solid support has preferably to be separated from the released material. The individual methods for this depend on the nature of the solid support and are known in the art. If the support material is contained within a column the released material can be collected as column flowthrough. In case the support material is mixed with the lysate components (so called batch procedure) an additional separation step such as gentle centrifugation may be necessary and the released material is collected as supernatant. Alternatively magnetic beads can be used as solid support so that the beads can be eliminated from the sample by using a magnetic device.

Methods for the detection of proteins (and, therefore, also for HDAC or a HDAC protein complex) or for the determination of their amounts are known in the art and include physico-chemical methods such as protein sequencing (e.g. Edmann degradation), analysis by mass spectrometry methods or immunodetection methods employing antibodies directed against the HDAC.

According to a preferred embodiment of the methods according to the first, second and third aspect of the invention, the extent of the binding is determined by separating the HDAC protein complex from the ligand and subsequent detection of a component of the separated HDAC protein complex or subsequent determination of the amount of a component of the separated HDAC protein complex.

This preferred embodiment is especially advantageous, because it ensures that only HDAC protein complex is detected and not also free HDAC which has been bound to the ligand. In this context, the component may be any component known to be part of the respective HDAC complex which binding to the ligand is to be assessed. As shown in Example 5 and FIGS. 27, 28 and 29, the detection of a component of the HDAC protein complex is especially suitable for identifying an HDAC interacting compound according to the methods of the invention. Consequently, in a preferred embodiment, the term “component of an HDAC protein complex” denotes a component of said complex which is not an HDAC protein.

Throughout the invention, if an antibody is used in order to detect a respective protein (e.g. HDAC or a component of the HDAC complex), a specific antibody may be used (Wu and Olson, 2002. J. Clin. Invest. 109(10):1327-1333). As indicated above, such antibodies are known in the art. Furthermore, the skilled person is aware of methods for producing the same.

Preferably, a mass spectrometry or immunodetection methods are used in the context of the methods of the invention.

The identification of proteins with mass spectrometric analysis (mass spectrometry) is known in the art (Shevchenko et al., 1996, Analytical Chemistry 68: 850-858; Mann et al., 2001, Analysis of proteins and proteomes by mass spectrometry, Annual Review of Biochemistry 70, 437-473) and is further illustrated in the example section.

Preferably, the mass spectrometry analysis is performed in a quantitative manner, for example by using iTRAQ technology (isobaric tags for relative and absolute quantification) or cICAT (cleavable isotope-coded affinity tags) (Wu et al., 2006. J. Proteome Res. 5, 651-658).

According to a further preferred embodiment of the present invention, the characterization by mass spectrometry (MS) is performed by the identification of proteotypic peptides of the HDAC. The idea is that the HDAC or the component of the HDAC complex is digested with proteases and the resulting peptides are determined by MS. As a result, peptide frequencies for peptides from the same source protein differ by a great degree, the most frequently observed peptides that “typically” contribute to the identification of this protein being termed “proteotypic peptide”. Therefore, a proteotypic peptide as used in the present invention is an experimentally well observable peptide that uniquely identifies a specific protein or protein isoform.

According to a preferred embodiment, the characterization is performed by comparing the proteotypic peptides obtained in the course of practicing the methods of the invention with known proteotypic peptides. Since, when using fragments prepared by protease digestion for the identification of a protein in MS, usually the same proteotypic peptides are observed for a given HDAC or component of the HDAC complex, it is possible to compare the proteotypic peptides obtained for a given sample with the proteotypic peptides already known for HDACs or the component and thereby identifying the HDAC or the component being present in the sample.

Suitable immunodetection methods include but are not limited to Western blots, ELISA assays, sandwich ELISA assays and antibody arrays or a combination thereof. The establishment of such assays is known in the art (Chapter 11, Immunology, pages 11-1 to 11-30 in: Short Protocols in Molecular Biology. Fourth Edition, Edited by F. M. Ausubel et al., Wiley, New York, 1999).

These assays can not only be configured in a way to detect and quantify a HDAC interacting protein of interest, but also to analyse posttranslational modification patterns of HDAC or of other components of the HDAC protein complex such as phosphorylation or ubiquitin modification.

As detailed above, the identification methods of the invention involve the use of compounds which are tested for their ability to be a HDAC interacting compound.

Principally, according to the present invention, such a compound can be every molecule which is able to interact with the HDAC, for example by inhibiting its binding to the immobilization product of the invention. Preferably, the compound has an effect on the HDAC, e.g. a stimulatory or inhibitory effect.

Preferably, said compound is selected from the group consisting of synthetic or naturally occurring chemical compounds or organic synthetic drugs, more preferably small molecule organic drugs or natural small molecule compounds. Preferably, said compound is identified starting from a library containing such compounds. Then, in the course of the present invention, such a library is screened.

Such small molecules are preferably not proteins or nucleic acids. Preferably, small molecules exhibit a molecular weight of less than 1000 Da, more preferred less than 750 Da, most preferred less than 500 Da.

A “library” according to the present invention relates to a (mostly large) collection of (numerous) different chemical entities that are provided in a sorted manner that enables both a fast functional analysis (screening) of the different individual entities, and at the same time provide for a rapid identification of the individual entities that form the library. Examples are collections of tubes or wells or spots on surfaces that contain chemical compounds that can be added into reactions with one or more defined potentially interacting partners in a high-throughput fashion. After the identification of a desired “positive” interaction of both partners, the respective compound can be rapidly identified due to the library construction. Libraries of synthetic and natural origins can either be purchased or designed by the skilled artisan.

Examples of the construction of libraries are provided in, for example, Breinbauer R, Manger M, Scheck M, Waldmann H. Natural product guided compound library development. Curr. Med. Chem. 2002; 9(23):2129-2145, wherein natural products are described that are biologically validated starting points for the design of combinatorial libraries, as they have a proven record of biological relevance. This special role of natural products in medicinal chemistry and chemical biology can be interpreted in the light of new insights about the domain architecture of proteins gained by structural biology and bioinformatics. In order to fulfill the specific requirements of the individual binding pocket within a domain family it may be necessary to optimise the natural product structure by chemical variation. Solid-phase chemistry is said to become an efficient tool for this optimisation process, and recent advances in this field are highlighted in this review article. The current drug discovery processes in many pharmaceutical companies require large and growing collections of high quality lead structures for use in high throughput screening assays. Collections of small molecules with diverse structures and “drug-like” properties have, in the past, been acquired by several means: by archive of previous internal lead optimisation efforts, by purchase from compound vendors, and by union of separate collections following company mergers. Although high throughput/combinatorial chemistry is described as being an important component in the process of new lead generation, the selection of library designs for synthesis and the subsequent design of library members has evolved to a new level of challenge and importance. The potential benefits of screening multiple small molecule compound library designs against multiple biological targets offers substantial opportunity to discover new lead structures.

In a preferred embodiment of the methods of the invention, the HDAC or HDAC protein complex containing protein preparation is first incubated with the compound and then with the ligand. However, the simultaneous incubation is equally preferred (competitive binding assay).

In case that the incubation with the compound is first, the HDAC or HDAC protein complex containing protein preparation is preferably first incubated with the compound for 10 to 60 minutes, more preferred 30 to 45 minutes at a temperature of 4° C. to 37° C., more preferred 4° C. to 25° C., most preferred 4° C. Preferably compounds are used at concentrations ranging from 1 nM to 100 μM, preferably from 10 nM to 10 μM. The second step, contacting with the ligand, is preferably performed for 10 to 60 minutes at 4° C.

In case of simultaneous incubation, the HDAC or HDAC protein complex containing protein preparation is preferably simultaneously incubated with the compound and the immobilization product of the invention for 30 to 120 minutes, more preferred 60 to 120 minutes at a temperature of 4° C. to 37° C., more preferred 4° C. to 25° C., most preferred 4° C. Preferably compounds are used at concentrations ranging from 1 nM to 100 μM, preferably from 10 nM to 10 μM.

Furthermore, the methods of the invention may be performed with several protein preparations in order to test different compounds. This embodiment is especially interesting in the context of medium or high throughput screenings.

Preferably, the identification methods of the invention are performed as a medium or high throughput screening.

The interaction compound identified according to the present invention may be further characterized by determining whether it has an effect on the HDAC, for example on its HDAC activity (Khan et al., 2008. Biochem. J. 409(2):581-9; Blackwell et al., 2008. Life Sci. 82(21-22):1050-1058).

The compounds identified according to the present invention may further be optimized (lead optimisation). This subsequent optimisation of such compounds is often accelerated because of the structure-activity relationship (SAR) information encoded in these lead generation libraries. Lead optimisation is often facilitated due to the ready applicability of high-throughput chemistry (HTC) methods for follow-up synthesis. An example for lead optimization of HDAC inhibitors was reported (Remiszewski et al., 2003. J. Med. Chem. 46(21):4609-4624).

The invention further relates to a method for the preparation of a pharmaceutical composition comprising the steps of
a) identifying a HDAC interacting compound as described above, and
b) formulating the interacting compound to a pharmaceutical composition.

Methods for the formulation of identified compounds are known in the art. Furthermore, it is known in the art how to administer such pharmaceutical compositions.

The obtained pharmaceutical composition can be used for the prevention or treatment of diseases where the respective HDAC plays a role, e.g. for the prevention or treatment of cancer (Bolden et al., 2006. Nat. Rev. Drug Discov. 5(9):769-784). For example, HDAC inhibitors may be useful for the treatment of inflammatory diseases, cancer or neurodegenerative diseases.

The invention is further described by the following figures and examples, which are intended to illustrate, but not to limit the present invention. In case where in the following examples the term “affinity matrix” is used, this term refers to the immobilized ligand as defined in the present application.

### SHORT DESCRIPTION OF THE FIGURES

FIG. 1: Synthesis of the immobilization compound. Steps: i) DCC, HOBt, DMF, rt, ii) NaOHaq 4N, MeOH, iii) H2NOCH2Ph.HCl, PyBrop, DIEA, DMF, iv) 10% Pd/C, EtOH, v) 4N HCl in Dioxane. Methods used in the synthesis of the immobilization compound are described in example 1.

FIG. 2: Structure of the immobilization compound based on SAHA.

FIG. 3: Amino acid sequence of human HDAC1 (IPI00013774.1). Peptides identified by mass spectrometry are underlined (K562; experiment X003787).

FIG. 4: Amino acid sequence of human HDAC2 (IPI00289601.10). Peptides identified by mass spectrometry are underlined (K562; experiment X003787).

FIG. 5: Amino acid sequence of human HDAC3 (IPI00217965.1). Peptides identified by mass spectrometry are underlined (K562; experiment X003787).

FIG. 6: Amino acid sequence of human HDAC6 (W100005711.4). Peptides identified by mass spectrometry are underlined (K562; experiment X003787).

FIG. 7: Amino acid sequence of human HDAC8 (IPI00747259.2). Peptides identified by mass spectrometry are underlined (K562; experiment X003787).

FIG. 8: Amino acid sequence of human HDAC10 (IPI00012439.5). Peptides identified by mass spectrometry are underlined (K562; experiment X003787).

FIG. 9: Amino acid sequence of human AOF2/LSD1 (IPI00217540.7). Peptides identified by mass spectrometry are underlined (K562; experiment X003787).

FIG. 10: Amino acid sequence of human GSE1 (IPI00215963.5). Peptides identified by mass spectrometry are underlined (K562; experiment X003787).

FIG. 11: Amino acid sequence of human HMG20A (IPI00018924.3). Peptides identified by mass spectrometry are underlined (K562; experiment X003787).

FIG. 12: Amino acid sequence of human HMG20B (W100464951.5). Peptides identified by mass spectrometry are underlined (K562; experiment X003787).

FIG. 13: Amino acid sequence of human RCOR1 (IPI00008531.1). Peptides identified by mass spectrometry are underlined (K562; experiment X003787).

FIG. 14: Amino acid sequence of human RCOR3 (IPI00914887.1). Peptides identified by mass spectrometry are underlined (K562; experiment X003787).

FIG. 15: Amino acid sequence of human ZNYM2 (IPI00294603.6). Peptides identified by mass spectrometry are underlined (K562; experiment X003787).

FIG. 16: Amino acid sequence of human GATAD2A (IPI00478128.2). Peptides identified by mass spectrometry are underlined (K562; experiment X003787).

FIG. 17: Amino acid sequence of human GATAD2B (IPI00103554.1). Peptides identified by mass spectrometry are underlined (K562; experiment X003787).

FIG. 18: Amino acid sequence of human MBD3 (IPI00439194.1). Peptides identified by mass spectrometry are underlined (K562; experiment X003787).

FIG. 19: Amino acid sequence of human MTA2 (IPI00171798.1). Peptides identified by mass spectrometry are underlined (K562; experiment X003787).

FIG. 20: Amino acid sequence of human MTA3 (IPI00165357.4). Peptides identified by mass spectrometry are underlined (K562; experiment X003787).

FIG. 21: Amino acid sequence of human SMARCC2 (IPI00216047.3). Peptides identified by mass spectrometry are underlined (K562; experiment X003787).

FIG. 22: Amino acid sequence of human TBL1X (IPI00640917.1). Peptides identified by mass spectrometry are underlined (K562; experiment X003787).

FIG. 23: Amino acid sequence of human TBL1XR1 (IPI00002922.5). Peptides identified by mass spectrometry are underlined (K562; experiment X003787).

FIG. 24: Amino acid sequence of human SIN3A (IPI00170596.1). Peptides identified by mass spectrometry are underlined (K562; experiment X003787).

FIG. 25: Amino acid sequence of human RBBP4 (IPI00328319.8). Peptides identified by mass spectrometry are underlined (K562; experiment X003787).

FIG. 26: Amino acid sequence of human RBBP7 (IPI00646512.1). Peptides identified by mass spectrometry are underlined (K562; experiment X003787).

FIG. 27: Dose response curves for HDACs (Example 5). HDAC1: IC₅₀=0.78 μM; HDAC2: IC₅₀=2.8 μM; HDAC6: IC₅₀=0.20 μM.

FIG. 28: Dose response curves for components of the Co-REST protein complex (Example 5): RCOR1: IC₅₀=0.15 μM; RCOR3: IC₅₀=0.15 μM; AOF2: IC₅₀=0.14 μM.

FIG. 29: Dose response curves for components of the NuRD protein complex (Example 5). MTA2: IC₅₀=0.53 μM; RBBP4: IC₅₀=0.46 μM; MTA3: IC₅₀=1.8 μM.

FIG. 30: Dose response curves for components of the NCoR protein complex (Example 7). HDAC3: IC₅₀=73 μM; TBL1X: IC₅₀=78 μM; TBL1XR1 (IC₅₀=60 μM).

FIG. 31: Mapping of HDAC drug target complexes in chemical space and in proteome space. (a) Chemoproteomics competition binding assay to profile HDAC inhibitor target complexes in cell extract. (I) Generation of a probe matrix by the derivatization of sepharose with analogues of nonselective HDACi (SAHA, ITF2357). (II) Cell extract aliquots are incubated with vehicle or with drug over a range of concentrations. (III) The “free” drug competes with the immobilized probes for the drug binding sites of the target protein complexes. (IV) Captured proteins are trypsinized and each peptide mixture is tagged with a distinct isobaric TMT tag. (V) Tagged samples are pooled and analyzed by LC-MS/MS such that each peptide gives rise to six reporter signals in the MS/MS spectrum. (VI) When capturing of a protein is competed away by the drug, a decrease of signal intensity compared to the vehicle control is detected for each peptide originating from this protein. Protein complexes formed by the target and associated proteins are defined by matching inhibition (IC₅₀) curves. (b) Definition of target protein complexes in biological space by quantitative immunoaffinity purification. Data are generated from the same cell extracts and are used to deconvolute protein complexes formed around the drug target.

FIG. 32: HDACi drug targets and target complexes are defined by chemoproteomic profiling of clinical and tool compounds. (a) Representative concentration-inhibition profiles of SAHA, BML-210 (the benzamide analog of SAHA), tacedinaline (CI-994), belinostat, and romidepsin were determined in K562 cell extract as outlined in FIG. 31. For clarity, selected profiles are grouped in 3 plots for each inhibitor: HDACs (left), components of CoREST, NuRD, Sin3, and NCoR complexes (middle), and other proteins either representing novel direct targets or complex components (right). (b) Assay reproducibility. Mean pIC₅₀ data for Trichostatin A from seven independent replicates are shown and statistically significant differences between potencies observed for individual HDAC1/2 complexes are indicated (**: p<0.01; ***: p<0.001). (c) Binding profiles for HDACs and protein complexes across a panel of 16 inhibitors. For each compound, its relative potency for each affected protein was calculated in relation to its most potently inhibited target as (pIC₅₀-min(pIC₅₀))/(max(pIC₅₀)-min(pIC₅₀)). The profile for each protein versus all compounds is traced by a grey lines with proteins of interest highlighted in different panels: class I HDACs, class II HDACS, CoREST components, NCoR components, NuRD components, and Sin3 components. Putative novel HDAC complexes formed around MIDEAS (mitotic deacetylase complex, MiDAC) and other ELSA (ELM-SANT) domain proteins are represented. (d) Bidirectional hierarchic clustering of the concentration-inhibition (relative potency) data for 16 inhibitors versus 344 proteins (each targeted by at least one inhibitor). For readability the parts of the clustering around HDAC targets is represented at a higher vertical resolution. Previously published associations of proteins to complexes are represented.

FIG. 33: Deconvolution of protein complexes by IP-MS/MS analysis confirms novel HDAC complexes. (a) Selected probe matrix interactome proteins identified in IP-MS/MS analysis of HDAC complexes. IPs were performed in K562 cells using antibodies directed against HDAC1, 2, and 3, known complex subunits (the CoREST subunit LSD1; the NuRD subunit MTA3; the Sin3 subunit SIN3A; and the NCoR-subunit TBL1XR1), and examples of novel HDACi binding proteins (DNTTIP1 and TRERF1). * denotes previously reported complex components not identified in the matrix interactome. (b) Examples of the quantitative mapping of immunoaffinity-purified protein complexes by MS/MS. Purifications conducted with two different antibodies each and their corresponding isotype controls were combined after PAGE purification, trypsinisation and isobaric tagging. Protein quantification data are represented as plots of relative enrichment in immunoprecipitates of (upper panel) Sin3 vs. LSD1, and (lower panel) MTA3 vs. TBL1XR1. Each square represents a protein with its size scaled according to the number of sequence-to-spectrum matches. (c) HDAC protein complexes in chemical and biological space. The enrichment of proteins in the IP samples is plotted against the inhibitor potency data.

FIG. 34: Differential effects of HDAC inhibitors on histone and tubulin acetylation. (a) Immunofluorescence analysis of histone H3 (K9ac/K14ac) and tubulin acetylation in HeLa cells treated for 4 hours with vehicle, SAHA (10 μM), tacedinaline (50 μM), PCI-24781 (20 μM), PCI-34051 (100 μM), romidepsin (1 μM) or valproate (2000 μM). (b) Mapping of histone acetylation by LC-MS/MS of K562 cells treated with HDACi. Cells were treated with TSA (10 μM), SAHA (5 μM), PCI-24781 (2 μM), tacedinaline (50 μM), romidepsin (1 μM), PCI-34051 (20 μM), bufexamac (100 μM), MC 1293 (100 μM), or valproate (2000 μM) for 6 hours. Histones were extracted from cells, trypsinized, and acetylated peptides were quantified after isobaric mass (TMT) tagging. Heat map representation of the abundance of histone peptides with single or multiple acetylated lysines as a result of inhibitor treatment and bar chart representation of abundance of differently modified variants of the Histone H3.3 peptide 9-17.

FIG. 35: The non-steroidal anti-inflammatory drug bufexamac is a novel Class IIb HDAC inhibitor. (a) Screen of a focused compound library against HDACs 1, 2, 3 and 6 using a chemoproteomics binding assay with the SAHA matrix in whole cell extract from Jurkat and Ramos cells. The plots outline inhibition relative to HDAC1 for HDAC6, HDAC3, and HDAC2, as quantified by antibodies on dot-blot arrays. The compound concentration was 10 μM and chemical structures of selective hit compounds are shown. (b) HDAC selectivity profile of bufexamac in K562 cells, measured as outlined in FIG. 31. (c) Treatment of HeLa cells with bufexamac elicits hyperacetylation of tubulin whereas treatment with the o-aminoanilide AA-2 leads to hyperacetylation of histones. Cultured cells were treated with vehicle or drug for 4 h, and cells were analyzed by immunofluorescence microscopy and by western blotting using antibodies for acetylated tubulin (EC₅₀=2.9 μM) and acetylated histones H3 (K9) and H4 (K5), respectively. (d) Treatment of peripheral blood mononuclear cells with bufexamac inhibits the secretion of IFNα (EC₅₀=8.9+/−4.9 μM, 3 independent experiments).

### EXAMPLES

### Example 1

### Preparation of the Affinity Matrix

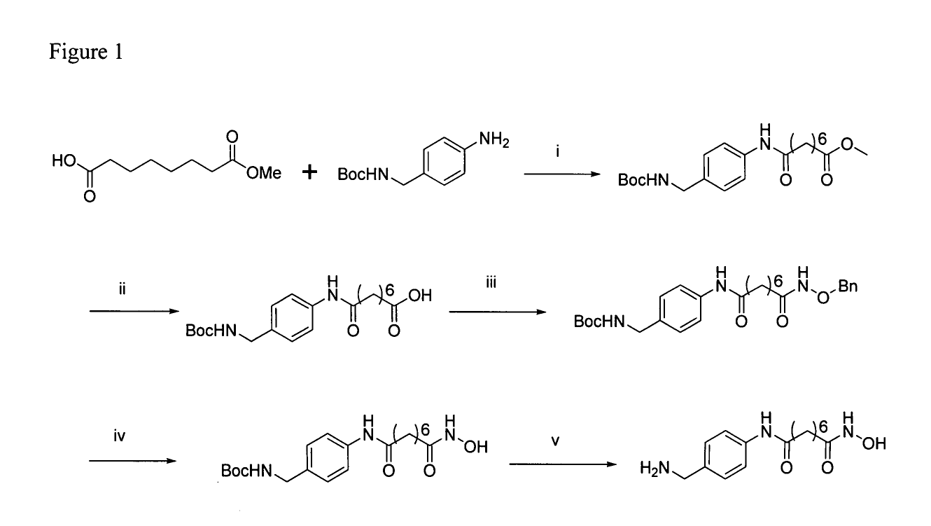

This example describes the synthesis of compounds (FIG. 1) and methods for their immobilization on a solid support yielding the affinity matrix used in the following examples for the capturing of HDACs from cell lysates.

### Synthesis of methyl 8-(4-((tert-butoxycarbonylamino)methyl)phenylamino)-8-oxooctanoate

To a stirred solution of suberic acid monomethyl ester (3.75 mmol, 1 eq, 0.706 g), 4[N-Boc aminomethyl]aniline (4.5 mmol, 1.2 eq, 1 g), Hydroxybenzotrizol (4.5 mmol, 1.2 eq, 0.608 g) in DMF (20 ml) was added dicyclocarbodiamide (4.5 mmol, 1.2 eq, 0.928 g). The reaction was stirred at room temperature overnight. The reaction precipitate was filtered. The filtrate was treated with water (20 ml). The resulting precipitate was filtered. The filtrate was then concentrated and purified by Flash Chromatography (Hexane/Ethyl acetate 1:1) to yield the desired compound as a white solid (m=1.25 g, 88%). LCMS (method A) Rt=2.93 nm, M+H=393, M+Na=415.

### Synthesis of 8-(4-((tert-butoxycarbonylamino)methyl)phenylamino)-8-oxooctanoic acid

To a stirred solution of 8-(4-((tert-butoxycarbonylamino)methyl)phenylamino)-8-oxooctanoate (0.579 mmol, 0.227 g) in methanol (7 ml) was added 2N aqueous sodium hydroxide (1.16 mmol, 2 eq, 0.580 ml). The reaction was stirred at room temperature overnight. The solvent was removed. The residue was dissolved in water. The pH was raised to 6 by addition of 2N aqueous hydrochloric acid. The resulting precipitate was filtered and dried in the vacuum oven (40° C.) overnight to yield the desired compound as a white solid (m=0.169 g, 77%) LCMS (Method B): Rt=2.48 nm M+H=379, M+Na=401

### Synthesis of tert-butyl 4-(8-(benzylaminooxy)-8-oxooctanamido)benzylcarbamate

To a stirred solution of 8-(4-((tert-butoxycarbonylamino)methyl)phenylamino)-8-oxooctanoic acid (0.397 mmo, 1 eq, 0.150 g) and O-benzylhydroxylamine hydrochloride (0.397 mmol, 1 eq, 0.063 g) in dimethylformamide (5 ml) and diisopropylethylamine (1.59 mmol, 4 eq, 0.277 ml) was added Bromo-tris-pyrrolidino phosphoniumhexafluorophosphate (0.595 mmol, 1.5 eq, 0.277 g). The reaction was stirred at room temperature overnight. The reaction was diluted with water (10 ml) and extracted with Ethyl acetate (2×50 ml). The organic layers were dried over magnesium sulphate, then concentrated and purified by flash chromatography (Hexane/Ethyl acetate (30-100%) to yield the desired product as an oil (m=0.105 g, 55%) LCMS (method B) rt=2.73 nm, M+H=484, M+Na=506.

### Synthesis of tert-butyl 4-(8-(aminooxy)-8-oxooctanamido)benzylcarbamate

To a degassed solution of tert-butyl 4-(8-(benzylaminooxy)-8-oxooctanamido)benzylcarbamate (0.787 mmol, 1 eq, 0.380 g) in ethanol (15 ml) was added 10% Pd/C (10%, 38 mg). The reaction was saturated in hydrogen and stirred under hydrogen atmosphere overnight. The catalyst was filtered and the filtrate concentrated to yield the desired compound as a yellow oil (0.309 g, 99%). LCMS (method C) RT=2.18 nm, M+H=394, M+Na=416.

### Synthesis of N-(4-(aminomethyl)phenyl)-8-(aminooxy)-8-oxooctanamide

To tert-butyl 4-(8-(aminooxy)-8-oxooctanamido)benzylcarbamate (0.22 mmol, 0.150 g) was added 2 ml of hydrochloric acid 4N in Dioxane. The mixture was stirred at room temperature for 3 hours. The reaction was evaporated and purified by HPLC (high pH) to yield the desired compound as an off white solid (m=51 mg, 46%). LCMS (method C): Rt=1.46 nm 2M+H=587, M+Na=316. ¹NMR (DMSO-d6, 400 MHz): 8=9.81 (s, 1H), 7.51 (d, 2H), 7.23 (d, 2H), 3.64 (s, 2H), 2.27 (d, 2H), 1.93 (d, 2H), 1.56 (m, 2H), 1.48 (m, 2H), 1.26 (m, 4H).

### LCMS Conditions

### Columns: Phenomenex Gemini-C18, 3.0×30 mm

Flow rate: 1.2 ml/mn

### Temperature: 40° C.

Wavelength: 254 nm (reference at 400 nm fro method B), 210 nm (reference at 360 nm for method B)

The mass spectrometry data were gathered in positive or negative mode, scanning for masses between 150 and 700 amu, using a fragmentor ramp (method B) from 118V to 400V (for MW=118.09 to MW=922.01 respectively) and a fragmentor set up to 100V for methods A and C.

### Solvents:

A=Water with 0.1% formic acid
B=Acetonitrile with 0.1% formic acid
C=Water with 0.1% ammonia
D=(95%:5%, acetonitrile:water) with 0.1% ammonia

### Gradient Conditions

TABLE 1
Method A: Short Column
Analytical, Low pH, Positive ion

| Time | | |
|---|---|---|
| (min) | % A | % B |
| 0.00 | 95.0 | 5.0 |
| 3.00 | 5.0 | 95.0 |
| 4.50 | 5.0 | 95.0 |
| 4.60 | 95.0 | 5.0 |
| 5.00 | 95.0 | 5.0 |

TABLE 2
Method B: Short Column
Analytical, Low pH, Positive ion

| Time | | |
|---|---|---|
| (min) | % A | % B |
| 0.00 | 95.0 | 5.0 |
| 3.00 | 5.0 | 95.0 |
| 4.50 | 5.0 | 95.0 |
| 4.60 | 95.0 | 5.0 |
| 5.00 | 95.0 | 5.0 |

TABLE 3
Method C: Short Column
Analytical, High pH, Positive ion

| Time | | |
|---|---|---|
| (min) | % C | % D |
| 0.00 | 95.0 | 5.0 |
| 3.00 | 0.0 | 100.0 |
| 4.50 | 0.0 | 100.0 |
| 4.60 | 95.0 | 5.0 |
| 5.00 | 95.0 | 5.0 |

### Conditions for the Prep HPLC High pH

### Column: Phenomenex Gemini C18 100×21.20 mm 5 μm

### Solvents:

A=Water+0.1% Ammonia
B=(95% Acetonitrile: 5% Water)+0.1% Ammonia
   Flow Rate: 20 ml/min
   Temperature: Room temperature
   Gradient conditions: The gradient conditions were variable depending on the retention time of each compound
   Wavelength: PDA detection from 200-400 nm (or 220 nm, depending on the purification system used)
   Mass spec conditions: The mass spec data were gathered in positive and negative mode, from 150 to 700 amu.

TABLE 4
Abbreviations

| | |
|---|---|
| Boc | tert-butoxycarbonyl |
| DCM | Dichloromethane |
| DMSO | dimethylsulfoxide |
| MeOH | Methanol |
| EtOH | Ethanol |
| ⁱPr2NEt | Diisopropylethylamine |
| NH₂OH•HCl | hydroxylaminehydrochloride |
| Pd(dppf)(Cl)₂ | [1,1′bis(diphenylphosphino) |
| | ferrocene] dichioro-palladium (II) |
| DMF | N,N-Dimethylformamide |
| THF | tetrahydrofuran |
| s | singlet |
| d | Doublet |
| dd | Doubledoublet |
| br | Broad |
| mL | millilitres |
| L | litre |
| t | Triplet |
| m | Multiplet |
| Rt | Retention time |

### Immobilization of Compounds on Beads (Affinity Matrix)

NHS-activated Sepharose 4 Fast Flow (Amersham Biosciences, 17-0906-01) was equilibrated with anhydrous DMSO (Dimethylsulfoxid, Fluka, 41648, H20<=0.005%). 1 ml of settled beads was placed in a 15 ml Falcon tube, compound stock solution (usually 100 mM in DMF or DMSO) was added (final concentration 0.2-2 μmol/ml beads) as well as 15 μl of triethylamine (Sigma, T-0886, 99% pure). Beads were incubated at room temperature in darkness on an end-over-end shaker (Roto Shake Genie, Scientific Industries Inc.) for 16-20 hours. Coupling efficiency is determined by HPLC. Non-reacted NHS-groups were blocked by incubation with aminoethanol at room temperature on the end-over-end shaker over night. Beads were washed with 10 ml of DMSO and were stored in isopropanol at −20° C. These beads were used as the affinity matrix in the following examples. Control beads (no compound immobilized) were generated by blocking the NHS-groups by incubation with aminoethanol as described above.

### Example 2

### HDAC Experiment Using an Immobilized Compound and a K-562 Cell Lysate

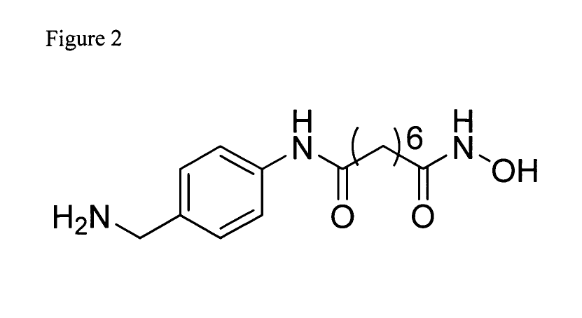

This example illustrates the use of a competition binding assay in cell lysate to identify and characterize HDAC protein complexes and to establish the selectivity profile of the test compound SAHA (vorinostat, suberoylanilide hydroxamic acid). This compound was added at defined concentrations (10 μM, 2.5 μM, 0.625 μM, 0.156 μM and 0.039 μM) to aliquots of K-562 cell lysates thereby allowing the test compound to bind to the target proteins in the lysate. Then the lysate was contacted with the affinity matrix (immobilized compound; FIG. 2) to capture remaining free target proteins. The proteins bound to the immobilized compound were eluted with detergent-containing buffer, separated on a SDS-polyacryamide gel and analyzed by mass spectrometry.

The peptide extracts corresponding to samples treated with different concentrations of the test compound (10 μM, 2.5 μM, 0.625 μM, 0.156 μM and 0.039 μM) and the solvent control (0.5% DMSO) were treated with different variants of the isobaric tagging reagent (TMT-reagents, Thermofisher). The TMT reagents are a set of multiplexed, amine-specific, stable isotope reagents that can label peptides in up to six different biological samples enabling simultaneous identification and quantitafication of peptides. The TMT-reagents reagents were used according to instructions provided by the manufacturer.

The combined samples were analyzed with a nano-flow liquid chromatography system coupled online to a tandem mass spectrometer (LC-MS/MS) experiment followed by reporter ion quantification in the MS/MS spectra (Ross et al., 2004. Mol. Cell. Proteomics 3(12):1154-1169; Dayon et al., 2008. Anal. Chem. 80(8):2921-2931; Thompson et al., 2003. Anal. Chem. 75(8):1895-1904). Further experimental protocols can be found in WO2006/134056 and a previous publication (Bantscheff et al., 2007. Nature Biotechnology 25, 1035-1044).

The test compound SAHA was used at five different concentrations in the cell lysate and the IC₅₀ values were normalized to the DMSO control. For selected HDACs and HDAC interactors the IC₅₀ values were plotted against the concentration of SAHA and curve fitting was performed using the Xlfit program (ID Business Solutions Ltd.) as previously described. (Bantscheff et al., 2007. Nature Biotechnology 25, 1035-1044). The IC₅₀ value corresponds to the test compound concentration at which the relative intensity of the MS signal for a protein is 50% compared to the solvent (DMSO) control.

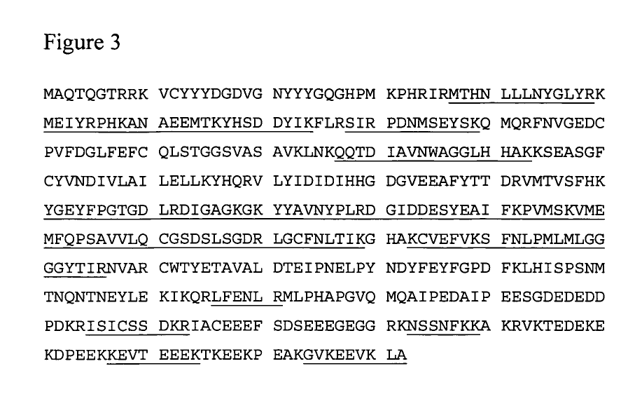

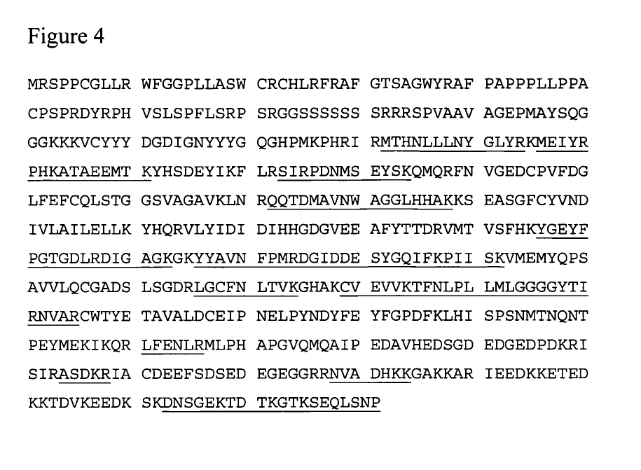

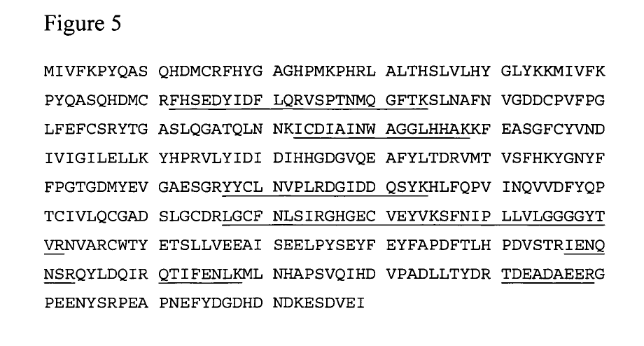

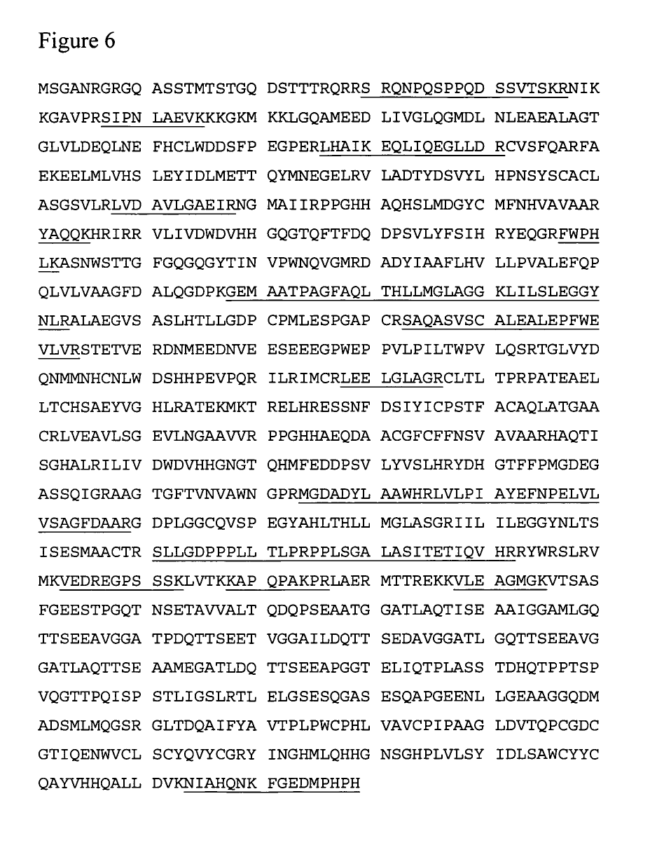

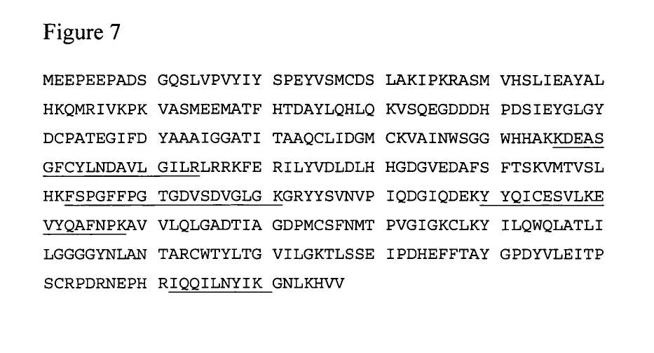

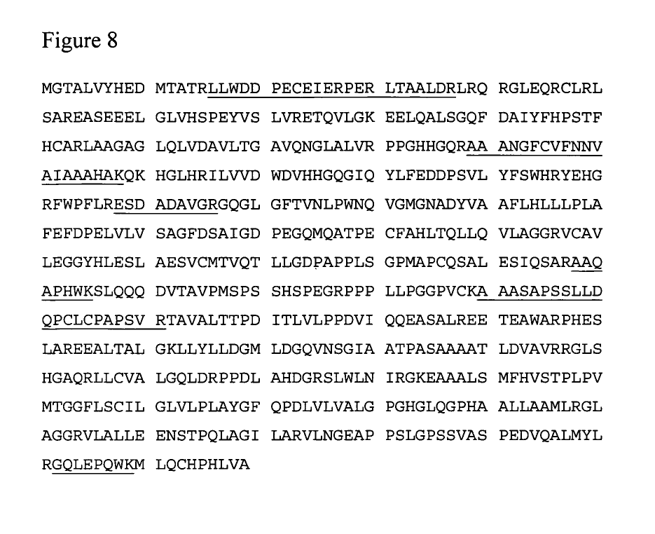

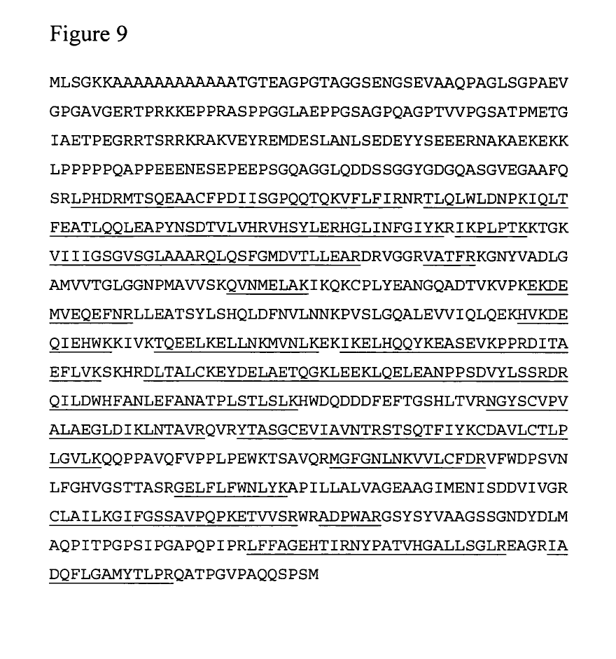

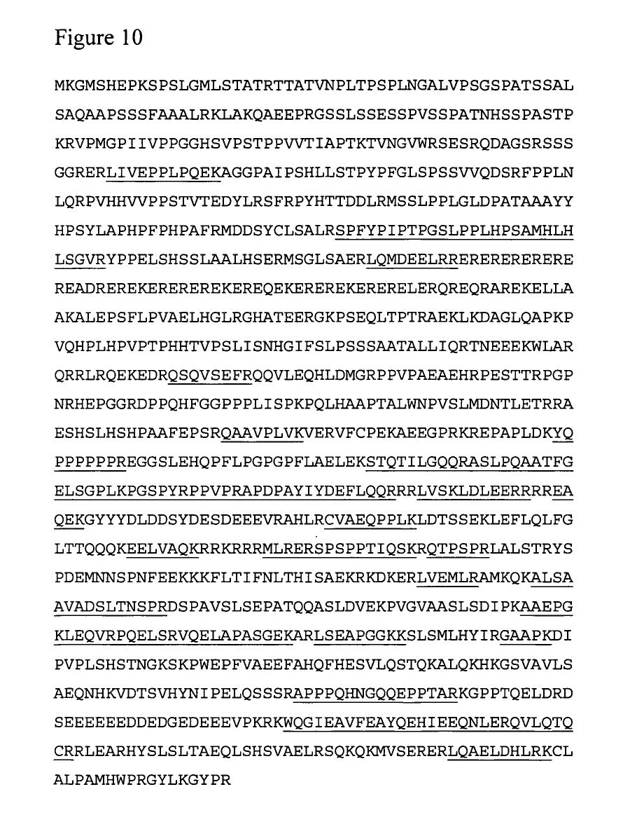

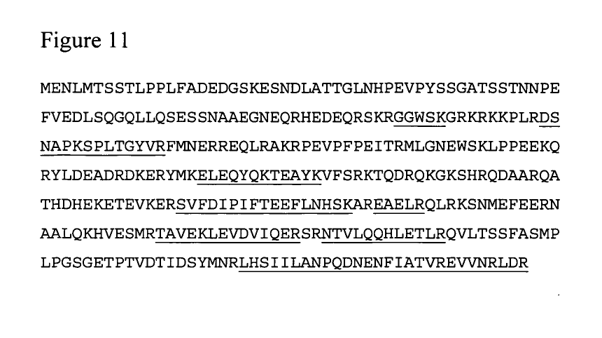

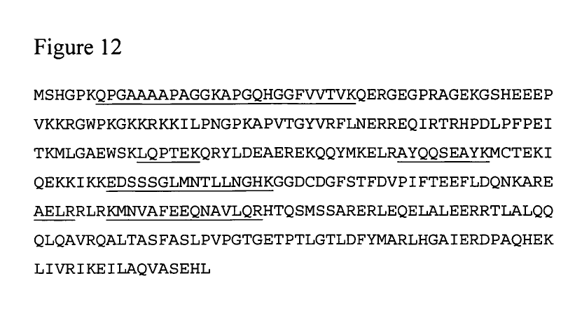

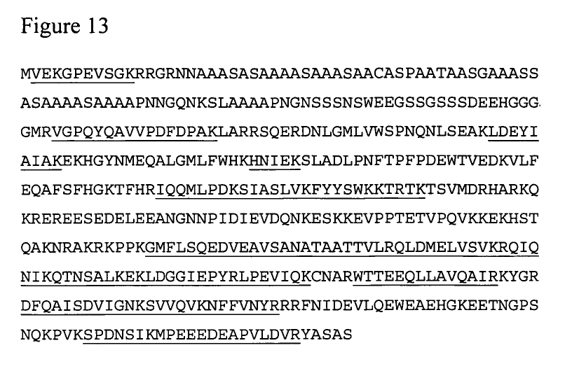

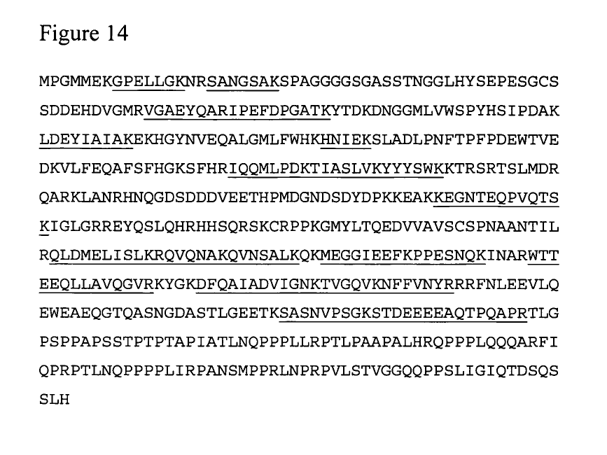

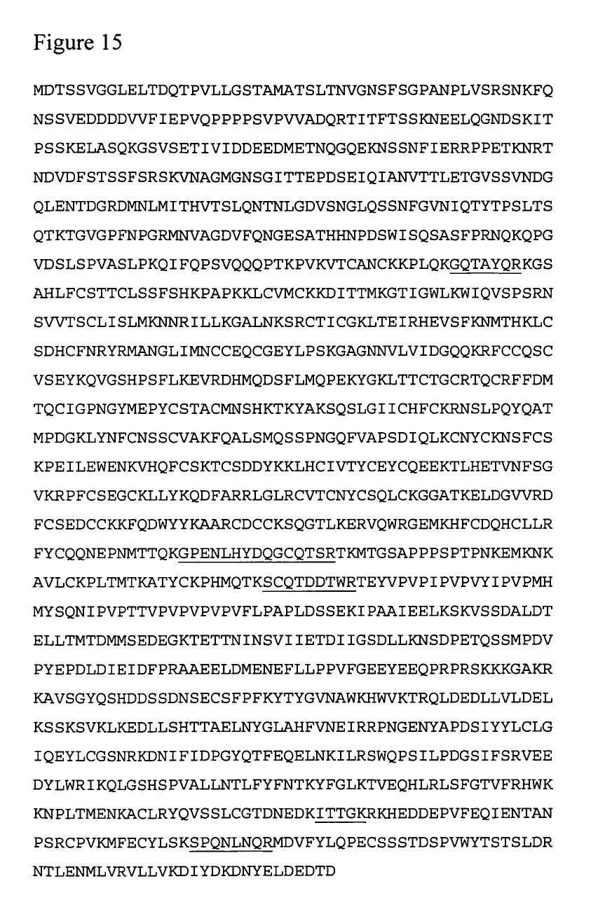

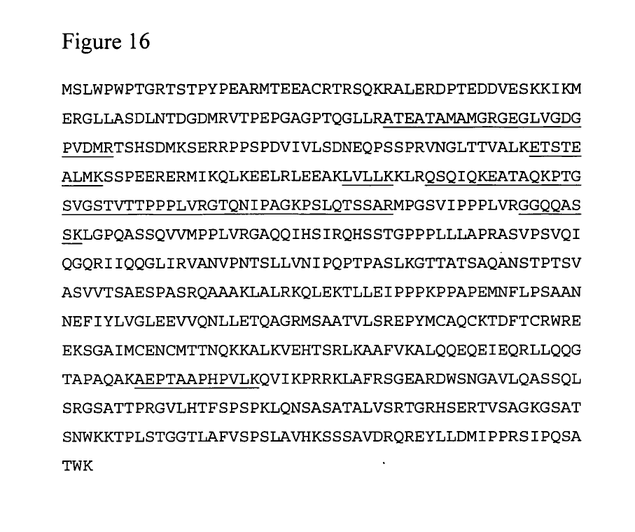

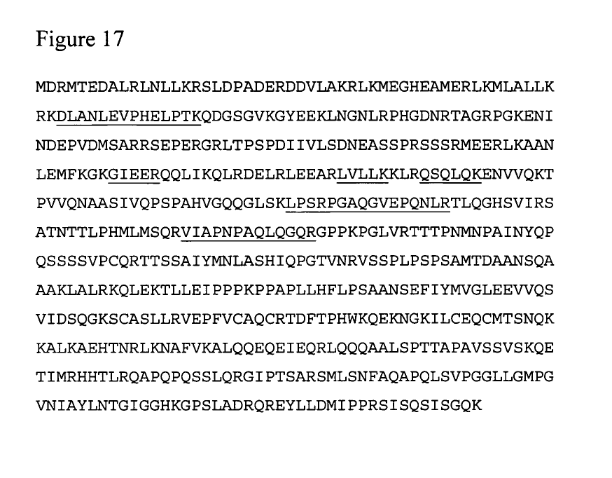

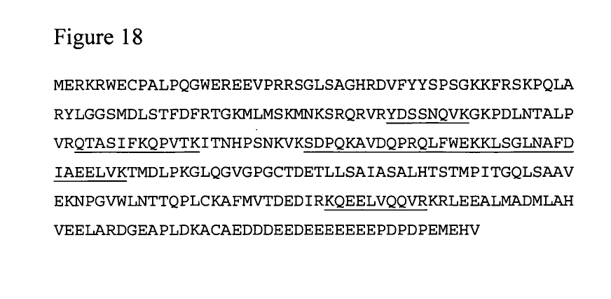

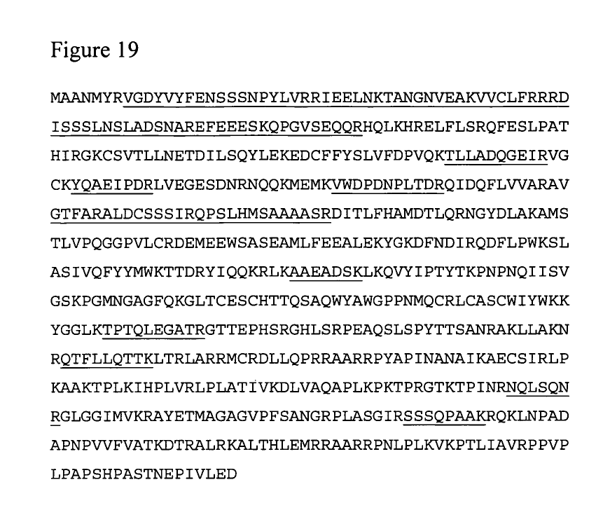

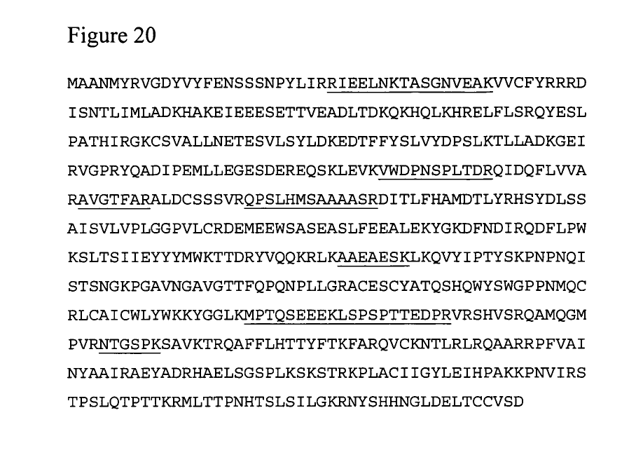

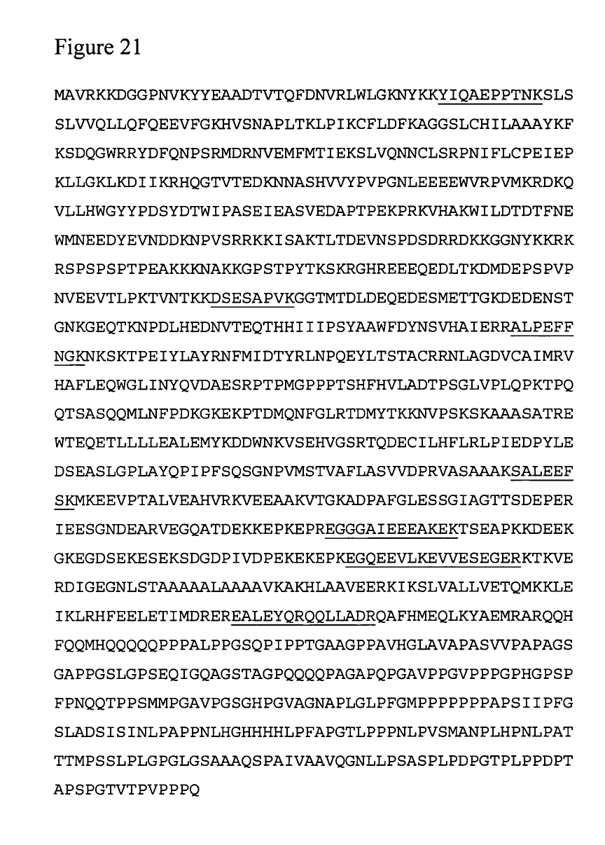

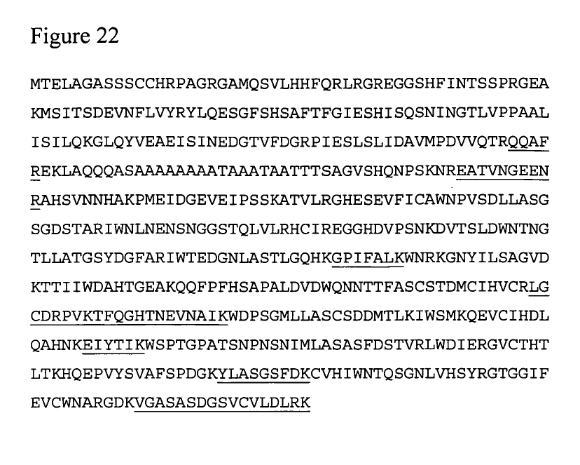

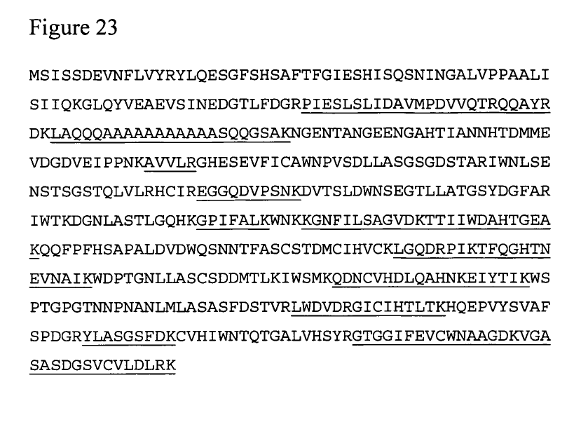

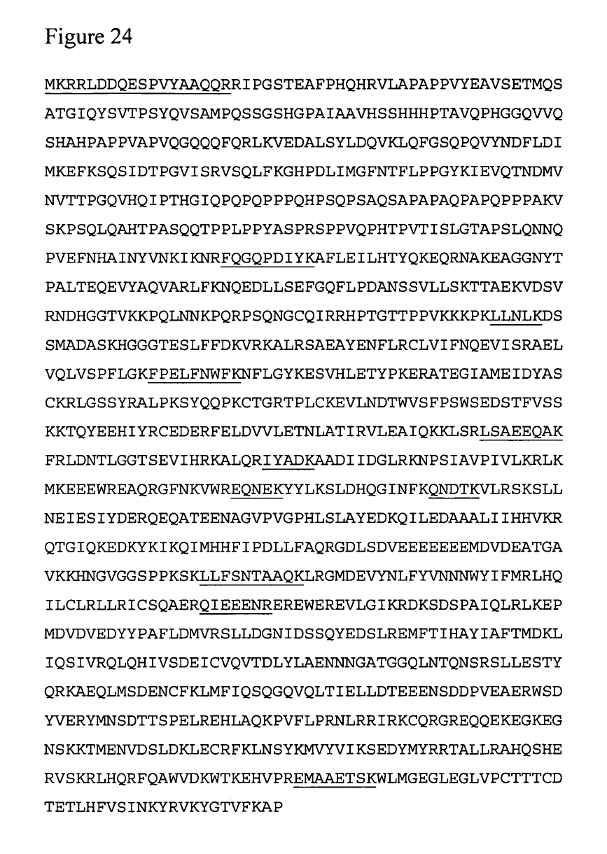

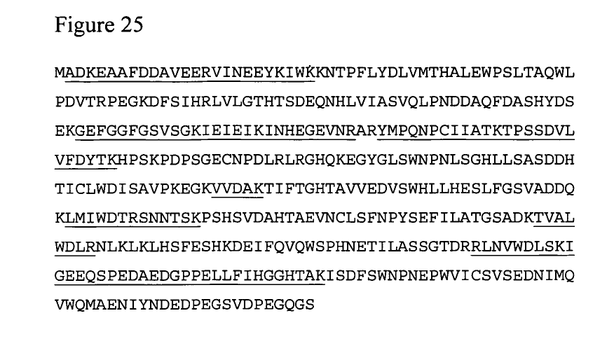

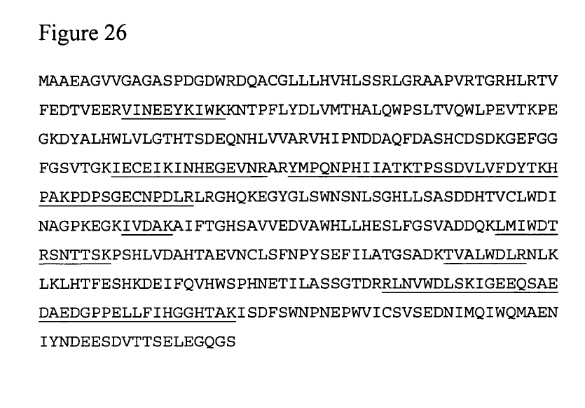

The identified HDACs and interactors are shown in Table 6 including the IC₅₀ values. In total six different HDACs were identified. For illustration, the identified peptides for HDAC1, HDAC2, HDAC3, HDAC6, HDAC8 and HDAC10 are shown in FIGS. 3 to 8. In addition, sequences of HDAC proteins complex components with idendified peptides underlined, are shown in FIGS. 9 to 26. Sequence identifiers are defined by the International Protein Index (IPI) (Kersey et al., 2004. Proteomics 4(7): 1985-1988).

### 1. Cell Culture

In this example K-562 cell lysate was used (Bantscheff et al., 2007. Nature Biotechnology 25, 1035-1044). K-562 cells (American Type Culture Collection-No. CCL-243) were either obtained from an external supplier (CIL SA, Mons, Belgium) or grown in one litre Spinner flasks (Integra Biosciences, #182101) in suspension in RPMI 1640 medium (Invitrogen, #21875-034) supplemented with 10% Fetal Bovine Serum (Invitrogen, #10270-106). Cells were harvested by centrifugation, washed once with 1×PBS buffer (Invitrogen, #14190-094) and cell pellets were frozen in liquid nitrogen and subsequently stored at −80° C.

### 2. Preparation of Cell Lysates

Cells were homogenized in a Potter S homogenizer in lysis buffer: 50 mM Tris-HCl, 0.8% NP40, 5% glycerol, 150 mM NaCl, 1.5 mM MgCl₂, 25 mM NaF, 1 mM sodium vanadate, 1 mM DTT, pH 7.5. One complete EDTA-free tablet (protease inhibitor cocktail, Roche Diagnostics, 1 873 580) per 25 ml buffer was added. The material was dounced 20 times using a mechanized POTTER S, transferred to 50 ml falcon tubes, incubated for 30 minutes rotating at 4° C. and spun down for 10 minutes at 20,000×g at 4° C. (10,000 rpm in Sorvall SLA600, precooled). The supernatant was transferred to an ultracentrifuge (UZ)-polycarbonate tube (Beckmann, 355654) and spun for 1 hour at 145.000×g at 4° C. (40.000 rpm in Ti50.2, precooled). The supernatant was transferred again to a fresh 50 ml falcon tube, the protein concentration was determined by a Bradford assay (BioRad) and samples containing 50 mg of protein per aliquot were prepared. The samples were immediately used for experiments or frozen in liquid nitrogen and stored frozen at −80° C.

3. Capturing of Proteins from Cell Lysate

Sepharose-beads with the immobilized compound (35 μl beads per pull-down experiment) were equilibrated in lysis buffer and incubated with a cell lysate sample containing 5 mg of protein on an end-over-end shaker (Roto Shake Genie, Scientific Industries Inc.) for 2 hours at 4° C. Beads were collected, transferred to Mobicol-columns (MoBiTech 10055) and washed with 10 ml lysis buffer containing 0.4% NP40 detergent, followed by 5 ml lysis buffer containing 0.2% detergent. To elute bound proteins, 60 μl 2×SDS sample buffer was added to the column. The column was incubated for 30 minutes at 50° C. and the eluate was transferred to a siliconized microfuge tube by centrifugation. Proteins were then alkylated with 108 mM iodoacetamid. Proteins were then separated by SDS-Polyacrylamide electrophoresis (SDS-PAGE).

### 4. Protein Identification by Mass Spectrometry

### 4.1 Protein Digestion Prior to Mass Spectrometric Analysis

Gel-separated proteins were digested in-gel essentially following a previously described procedure (Shevchenko et al., 1996, Anal. Chem. 68:850-858). Briefly, gel-separated proteins were excised from the gel using a clean scalpel, destained twice using 100 μl 5 mM triethylammonium bicarbonate buffer (TEAB; Sigma T7408) and 40% ethanol in water and dehydrated with absolute ethanol. Proteins were subsequently digested in-gel with porcine trypsin (Promega) at a protease concentration of 10 ng/μl in 5 mM TEAB. Digestion was allowed to proceed for 4 hours at 37° C. and the reaction was subsequently stopped using 5 μl 5% formic acid.

### 4.2 Sample Preparation Prior to Analysis by Mass Spectrometry

Gel plugs were extracted twice with 20 μl 1% formic acid and three times with increasing concentrations of acetonitrile. Extracts were subsequently pooled with acidified digest supernatants and dried in a vacuum centrifuge.

### 4.3 TMT Labeling of Peptide Extracts

The peptide extracts of samples treated with different concentrations of SAHA (10 μM, 2.5 μM, 0.625 μM, 0.156 μM and 0.039 μM) and the solvent control (0.5% DMSO) were treated with different variants of the isobaric tagging reagent (TMTsixplex Label Reagent Set, part number 90066, Thermo Fisher Scientific Inc., Rockford, Ill. 61105 USA). The TMT reagents are a set of multiplexed, amine-specific, stable isotope reagents that can label peptides on amino groups in up to six different biological samples enabling simultaneous identification and quantitation of peptides. The TMT reagents were used according to instructions provided by the manufacturer. The samples were resuspended in 10 μl 50 mM TEAB solution, pH 8.5 and 10 μl acetonitril were added. The TMT reagent was dissolved in acetonitril to a final concentration of 24 mM and 10 μl of reagent solution were added to the sample. The labeling reaction was performed at room temperature for one hour on a horizontal shaker and stopped by adding 5 μl of 100 mM TEAB and 100 mM glycine in water. The labeled samples were then combined, dried in a vacuum centrifuge and resuspended in 200 mM TEAB60%/40% acetonitril. 2 μl of a 2.5% NH2OH solution in water were added, incubated for 15 min and finally the reaction was stopped by addition of 10 μl of 20% formic acid in water. After freeze-drying samples were resuspended in 50 μl 0.1% formic acid in water.

### 4.4 Mass Spectrometric Data Acquisition

Peptide samples were injected into a nano LC system (CapLC, Waters or nano-LC 1D+, Eksigent) which was directly coupled either to a quadrupole TOF (QTOF Ultima, QTOF Micro, Waters), ion trap (LTQ) or Orbitrap mass spectrometer. Peptides were separated on the LC system using a gradient of aqueous and organic solvents (see below). Solvent A was 0.1% formic acid and solvent B was 70% acetonitrile in 0.1% formic acid.

TABLE 5
Peptides elution off the LC system

| | Flow | Gradient |
|---|---|---|
| | rate | Time(min)- |
| Method file | (nL/min) | % B |
| PQD_265min | 190 | 00-5.263 |
| | | 07-10 |
| | | 190-40.263 |
| | | 210-52.105 |
| | | 223-60 |
| | | 230-90 |
| | | 236-90 |
| | | 240-5.263 |
| | | 260-5.263 |

### 4.5 Protein Identification

The peptide mass and fragmentation data generated in the LC-MS/MS experiments were used to query a protein data base consisting of an in-house curated version of the International Protein Index (IPI) protein sequence database combined with a decoy version of this database (Elias and Gygi, 2007, Target-decoy search strategy for increased confidence in large-scale protein identifications by mass spectrometry. Nature Methods 4, 207-214). Proteins were identified by correlating the measured peptide mass and fragmentation data with data computed from the entries in the database using the software tool Mascot (Matrix Science; Perkins et al., 1999. Probability-based protein identification by searching sequence databases using mass spectrometry data. Electrophoresis 20, 3551-3567). Search criteria varied depending on which mass spectrometer was used for the analysis. Protein acceptance thresholds were adjusted to achieve a false discovery rate of below 1% as suggested by hit rates on the decoy data base (Elias and Gygi, 2007, Target-decoy search strategy for increased confidence in large-scale protein identifications by mass spectrometry. Nature Methods 4, 207-214).

### 4.6 Protein Quantitation

Relative protein quantitation was performed using peak areas of iTMT reporter ion signals essentially as described in an earlier publication (Bantscheff et al., 2007. Nature Biotechnology 25, 1035-1044).

TABLE 6
Selectivity profile for SAHA (K562 cells; experiment X003787)

| Representative | Protein | Protein | Protein | Redundant | Quantified | IC₅₀ |
|---|---|---|---|---|---|---|
| Sequence | Name | Complex | Function | Peptides | Spectra | (μM) |
| IPI00013774.1 | HDAC1 | CLASS I HDAC | HDAC | 133 | 86 | 0.90 |
| IPI00289601.10 | HDAC2 | CLASS I HDAC | HDAC | 232 | 76 | 0.93 |
| IPI00217965.1 | HDAC3 | CLASS I HDAC | HDAC | 120 | 38 | 2.56 |
| IPI00747259.2 | HDAC8 | CLASS I HDAC | HDAC | 51 | 17 | 10.00 |
| IPI00012439.5 | HDAC10 | CLASS II HDAC | HDAC | 51 | 14 | 10.00 |
| IPI00005711.4 | HDAC6 | CLASS II HDAC | HDAC | 83 | 81 | 0.32 |
| IPI00217540.7 | AOF2 | CoREST | HDAC Interactor | 214 | 191 | 0.54 |
| IPI00215963.5 | GSE1 | CoREST | HDAC Interactor | 306 | 88 | 0.64 |
| IPI00018924.3 | HMG20A | CoREST | HDAC Interactor | 33 | 21 | 0.35 |
| IPI00464951.5 | HMG20B | CoREST | HDAC Interactor | 17 | 13 | 1.20 |
| IPI00008531.1 | RCOR1 | CoREST | HDAC Interactor | 85 | 64 | 0.48 |
| IPI00914887.1 | RCOR3 | CoREST | HDAC Interactor | 164 | 60 | 0.39 |
| IPI00294603.6 | ZMYM2 | CoREST | HDAC Interactor | 15 | 13 | 1.67 |
| IPI00478128.2 | GATAD2A | Mi-2/NuRD | HDAC Interactor | 78 | 20 | 0.79 |
| IPI00103554.1 | GATAD2B | Mi-2/NuRD | HDAC Interactor | 18 | 8 | 2.50 |
| IPI00439194.1 | MBD3 | Mi-2/NuRD | HDAC Interactor | 40 | 19 | 0.61 |
| IPI00171798.1 | MTA2 | Mi-2/NuRD | HDAC Interactor | 42 | 29 | 10.00 |
| IPI00165357.4 | MTA3 | Mi-2/NuRD | HDAC Interactor | 64 | 4 | 1.93 |
| IPI00216047.3 | SMARCC2 | SMRT/NCoR | HDAC Interactor | 19 | 14 | 10.00 |
| IPI00640917.1 | TBL1X | SMRT/NCoR | HDAC Interactor | 56 | 4 | 0.29 |
| IPI00002922.5 | TBL1XR1 | SMRT/NCoR | HDAC Interactor | 55 | 29 | 0.29 |
| IPI00170596.1 | SIN3A | Sin3/CoREST | HDAC Interactor | 26 | 18 | 0.57 |
| IPI00328319.8 | RBBP4 | Sin3/Mi2-NuRD | HDAC Interactor | 130 | 29 | 0.79 |
| IPI00646512.1 | RBBP7 | Sin3/Mi2-NuRD | HDAC Interactor | 55 | 19 | 10.00 |
| IPI00006663.1 | ALDH2 | | | 195 | 151 | 0.63 |
| IPI00022305.4 | BZW2 | | | 108 | 31 | 3.07 |
| IPI00057097.3 | DNTTIP1 | | | 7 | 6 | 0.48 |
| IPI00784154.1 | HSPD1 | | | 152 | 132 | 10.00 |
| IPI00304082.8 | ISOC1 | | | 103 | 93 | 1.10 |
| IPI00003031.3 | ISOC2 | | | 106 | 74 | 0.24 |
| IPI00012833.1 | PPP4C | | | 55 | 32 | 10.00 |
| IPI00100933.1 | PTER | | | 90 | 38 | 10.00 |

### Example 3

### HDAC Experiment Using an Immobilized Compound and a Molt-4 Cell Lysate with Trichostatin A

This example illustrates the use of a competition binding assay in cell lysate to identify and characterize HDAC protein complexes and to establish the selectivity profile of the test compound Trichostatin A (Sigma-Aldrich). This compound was added at defined concentrations (10 μM, 2.5 μM, 0.625 μM, 0.156 μM and 0.039 μM) to aliquots of Molt-4 cell lysates thereby allowing the test compound to bind to the target proteins in the lysate. Then the lysate was contacted with the immobilized compound (FIG. 2) to capture remaining free target proteins. The proteins bound to the immobilized compound were eluted with detergent-containing buffer, separated on a SDS-polyacryamide gel and analyzed by mass spectrometry as described in example 2.

TABLE 7
Selectivity profile for Trichostatin A (Molt-4; experiment X003750)

| Representative | Protein | Protein | Protein | Redundant | Quantified | IC₅₀ |
|---|---|---|---|---|---|---|
| Sequence | Name | Complex | Function | Peptides | Spectra | (μM) |
| IPI00013774.1 | HDAC1 | CLASS I HDAC | HDAC | 308 | 98 | 0.04 |
| IPI00289601.10 | HDAC2 | CLASS I HDAC | HDAC | 259 | 80 | 0.05 |
| IPI00217965.1 | HDAC3 | CLASS I HDAC | HDAC | 153 | 49 | 0.09 |
| IPI00747259.2 | HDAC8 | CLASS I HDAC | HDAC | 33 | 11 | 3.00 |
| IPI00012439.5 | HDAC10 | CLASS II HDAC | HDAC | 42 | 7 | 3.00 |
| IPI00005711.4 | HDAC6 | CLASS II HDAC | HDAC | 110 | 94 | 0.40 |
| IPI00217540.7 | AOF2 | CoREST | HDAC Interactor | 292 | 134 | 0.02 |
| IPI00215963.5 | GSE1 | CoREST | HDAC Interactor | 195 | 55 | 0.02 |
| IPI00018924.3 | HMG20A | CoREST | HDAC Interactor | 14 | 9 | 0.03 |
| IPI00878364.1 | HMG20B | CoREST | HDAC Interactor | 8 | 8 | 0.02 |
| IPI00008531.1 | RCOR1 | CoREST | HDAC Interactor | 78 | 65 | 0.02 |
| IPI00914887.1 | RCOR3 | CoREST | HDAC Interactor | 106 | 37 | 0.02 |
| IPI00294603.6 | ZMYM2 | CoREST | HDAC Interactor | 14 | 12 | 0.04 |
| IPI00478128.2 | GATAD2A | Mi-2/NuRD | HDAC Interactor | 66 | 22 | 0.04 |
| IPI00103554.1 | GATAD2B | Mi-2/NuRD | HDAC Interactor | 22 | 13 | 0.06 |
| IPI00439194.1 | MBD3 | Mi-2/NuRD | HDAC Interactor | 60 | 24 | 0.04 |
| IPI00012773.1 | MTA1 | Mi-2/NuRD | HDAC Interactor | 108 | 4 | 0.06 |
| IPI00171798.1 | MTA2 | Mi-2/NuRD | HDAC Interactor | 50 | 25 | 0.06 |
| IPI00165357.4 | MTA3 | Mi-2/NuRD | HDAC Interactor | 168 | 19 | 0.03 |
| IPI00012301.1 | GPS2 | SMRT/NCoR | HDAC Interactor | 16 | 5 | 0.10 |
| IPI00640917.1 | TBL1X | SMRT/NCoR | HDAC Interactor | 80 | 4 | 0.02 |
| IPI00002922.5 | TBL1XR1 | SMRT/NCoR | HDAC Interactor | 86 | 44 | 0.07 |
| IPI00170596.1 | SIN3A | Sin3/CoREST | HDAC Interactor | 26 | 21 | 0.06 |
| IPI00328319.8 | RBBP4 | Sin3/Mi2-NuRD | HDAC Interactor | 122 | 24 | 0.05 |
| IPI00646512.1 | RBBP7 | Sin3/Mi2-NuRD | HDAC Interactor | 55 | 16 | 0.12 |
| IPI00006663.1 | ALDH2 | | | 38 | 36 | 3.00 |
| IPI00872929.2 | APOBEC3C | | | 10 | 5 | 3.00 |
| IPI00022305.4 | BZW2 | | | 58 | 26 | 2.45 |
| IPI00057097.3 | DNTTIP1 | | | 11 | 10 | 0.04 |
| IPI00784154.1 | HSPD1 | | | 124 | 114 | 3.00 |
| IPI00304082.8 | ISOC1 | | | 135 | 111 | 3.00 |
| IPI00006408.4 | NOSIP | | | 6 | 5 | 3.00 |
| IPI00012833.1 | PPP4C | | | 62 | 40 | 3.00 |
| IPI00100933.1 | PTER | | | 90 | 39 | 3.00 |

### Example 4

### HDAC Experiment Using an Immobilized Compound and a K-562 Cell Lysate with Trichostatin A

This example illustrates the use of a competition binding assay in cell lysate to identify and characterize HDAC protein complexes and to establish the selectivity profile of the test compound Trichostatin A (Sigma-Aldrich). This compound was added at defined concentrations (10 μM, 2.5 μM, 0.625 μM, 0.156 μM and 0.039 μM) to aliquots of K-562 cell lysates thereby allowing the test compound to bind to the target proteins in the lysate. Then the lysate was contacted with the immobilized compound (FIG. 2) to capture remaining free target proteins. The proteins bound to the immobilized compound were eluted with detergent-containing buffer, separated on a SDS-polyacryamide gel and analyzed by mass spectrometry as described in example 2.

TABLE 8
Selectivity profile for Trichostatin A (K-562; experiment X003754)

| Representative | Protein | Protein | Protein | Redundant | Quantified | IC₅₀ |
|---|---|---|---|---|---|---|
| Sequence | Name | Complex | Function | Peptides | Spectra | (μM) |
| IPI00013774.1 | HDAC1 | CLASS I | HDAC | 126 | 72 | 0.03 |
| | | HDAC | | | | |
| IPI00289601.10 | HDAC2 | CLASS I | HDAC | 256 | 81 | 0.05 |
| | | HDAC | | | | |
| IPI00217965.1 | HDAC3 | CLASS I | HDAC | 114 | 34 | 0.13 |
| | | HDAC | | | | |
| IPI00747259.2 | HDAC8 | CLASS I | HDAC | 28 | 7 | 3.00 |
| | | HDAC | | | | |
| IPI00012439.5 | HDAC10 | CLASS II | HDAC | 45 | 14 | 3.00 |
| | | HDAC | | | | |
| IPI00005711.4 | HDAC6 | CLASS II | HDAC | 108 | 99 | 0.64 |
| | | HDAC | | | | |
| IPI00217540.7 | AOF2 | CoREST | HDAC | 175 | 157 | 0.03 |
| | | | Interactor | | | |
| IPI00215963.5 | GSE1 | CoREST | HDAC | 99 | 88 | 0.03 |
| | | | Interactor | | | |
| IPI00018924.3 | HMG20A | CoREST | HDAC | 42 | 28 | 0.02 |
| | | | Interactor | | | |
| IPI00464951.5 | HMG20B | CoREST | HDAC | 24 | 19 | 0.03 |
| | | | Interactor | | | |
| IPI00008531.1 | RCOR1 | CoREST | HDAC | 79 | 60 | 0.02 |
| | | | Interactor | | | |
| IPI00914887.1 | RCOR3 | CoREST | HDAC | 146 | 50 | 0.03 |
| | | | Interactor | | | |
| IPI00294603.6 | ZMYM2 | CoREST | HDAC | 24 | 10 | 0.06 |
| | | | Interactor | | | |
| IPI00478128.2 | GATAD2A | Mi-2/NuRD | HDAC | 57 | 17 | 0.06 |
| | | | Interactor | | | |
| IPI00103554.1 | GATAD2B | Mi-2/NuRD | HDAC | 12 | 10 | 0.06 |
| | | | Interactor | | | |
| IPI00439194.1 | MBD3 | Mi-2/NuRD | HDAC | 38 | 16 | 0.05 |
| | | | Interactor | | | |
| IPI00171798.1 | MTA2 | Mi-2/NuRD | HDAC | 11 | 7 | 0.07 |
| | | | Interactor | | | |
| IPI00165357.4 | MTA3 | Mi-2/NuRD | HDAC | 68 | 14 | 0.06 |
| | | | Interactor | | | |
| IPI00216047.3 | SMARCC2 | SMRT/NCoR | HDAC | 28 | 12 | 3.00 |
| | | | Interactor | | | |
| IPI00640917.1 | TBL1X | SMRT/NCoR | HDAC | 72 | 8 | 0.05 |
| | | | Interactor | | | |
| IPI00002922.5 | TBL1XR1 | SMRT/NCoR | HDAC | 57 | 21 | 0.07 |
| | | | Interactor | | | |
| IPI00170596.1 | SIN3A | Sin3/CoREST | HDAC | 16 | 14 | 0.10 |
| | | | Interactor | | | |
| IPI00328319.8 | RBBP4 | Sin3/Mi2- | HDAC | 128 | 30 | 0.05 |
| | | NuRD | Interactor | | | |
| IPI00646512.1 | RBBP7 | Sin3/Mi2- | HDAC | 144 | 12 | 0.18 |
| | | NuRD | Interactor | | | |
| IPI00006663.1 | ALDH2 | | | 293 | 248 | 3.00 |
| IPI00872929.2 | APOBEC3C | | | 30 | 15 | 3.00 |
| IPI00022305.4 | BZW2 | | | 84 | 35 | 2.79 |
| IPI00057097.3 | DNTTIP1 | | | 12 | 11 | 0.05 |
| IPI00784154.1 | HSPD1 | | | 160 | 143 | 3.00 |
| IPI00304082.8 | ISOC1 | | | 220 | 174 | 3.00 |
| IPI00003031.3 | ISOC2 | | | 237 | 171 | 3.00 |
| IPI00006408.4 | NOSIP | | | 7 | 4 | 3.00 |
| IPI00012833.1 | PPP4C | | | 27 | 16 | 3.00 |
| IPI00100933.1 | PTER | | | 100 | 43 | 3.00 |
| IPI00005492.2 | WDR5 | | | 6 | 6 | 0.42 |

### Example 5

### HDAC Experiment Using an Immobilized Compound and a Mix of Jurkat and Ramos cell Lysates with SAHA

This example illustrates the use of a competition binding assay in cell lysate to identify and characterize HDAC protein complexes and to establish the selectivity profile of the test compound SAHA (vorinostat, suberoylanilide hydroxamic acid). This compound was added at defined concentrations (10 μM, 2.5 μM, 0.625 μM, 0.156 μM and 0.039 μM) to aliquots of a mix of Jurkat and Ramos cell lysates thereby allowing the test compound to to bind to the target proteins in the lysate. Then the lysate was contacted with the immobilized compound (FIG. 2) to capture remaining free target proteins. The proteins bound to the immobilized compound were eluted with detergent-containing buffer, separated on a SDS-polyacryamide gel and analyzed by mass spectrometry as described in example 2.

For the preparation of lysates, Jurkat cells (ATCC number TIB-152) and Ramos cells (ATCC number CRL-1596) were either obtained from an external supplier (CIL SA, Mons, Belgium) or grown in one litre Spinner flasks (Integra Biosciences, #182101) in suspension in RPMI 1640 medium (Invitrogen, #21875-034) supplemented with 10% Fetal Bovine Serum (Invitrogen, #10270-106) at a density between 0.2×10⁶ and 1.0×10⁶ cells/ml. Cells were harvested by centrifugation, washed once with 1×PBS buffer (Invitrogen, #14190-094) and cell pellets were frozen in liquid nitrogen and subsequently stored at −80° C.

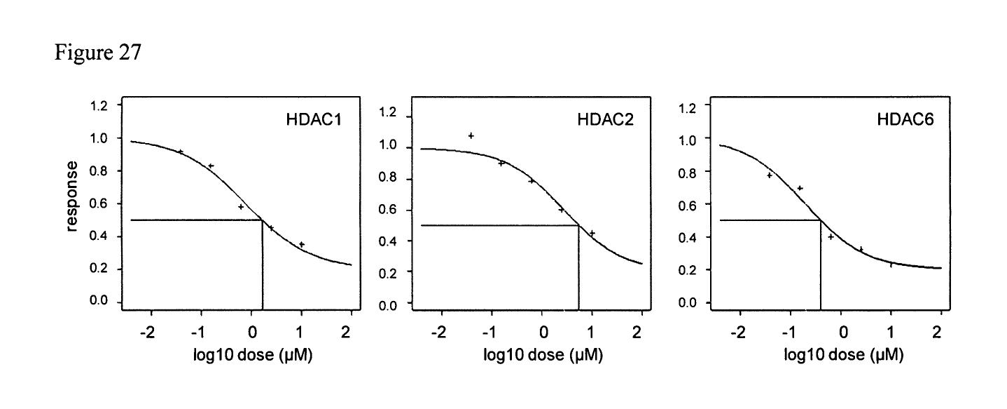

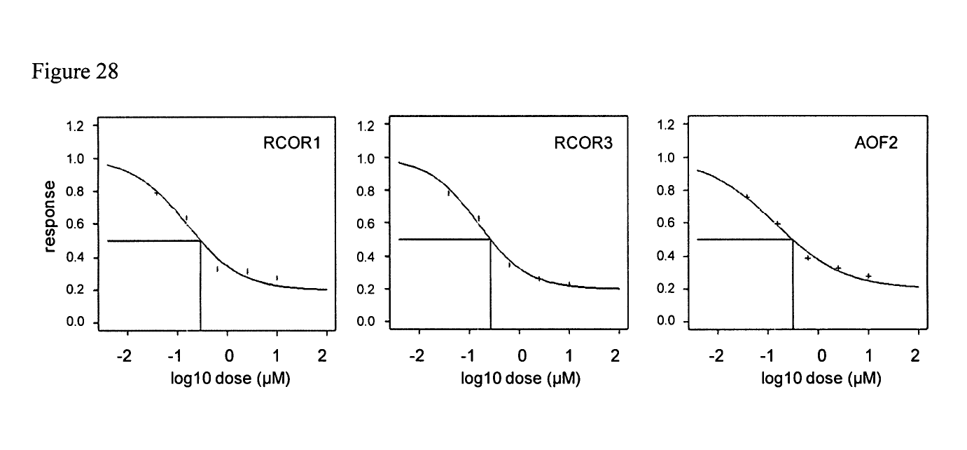

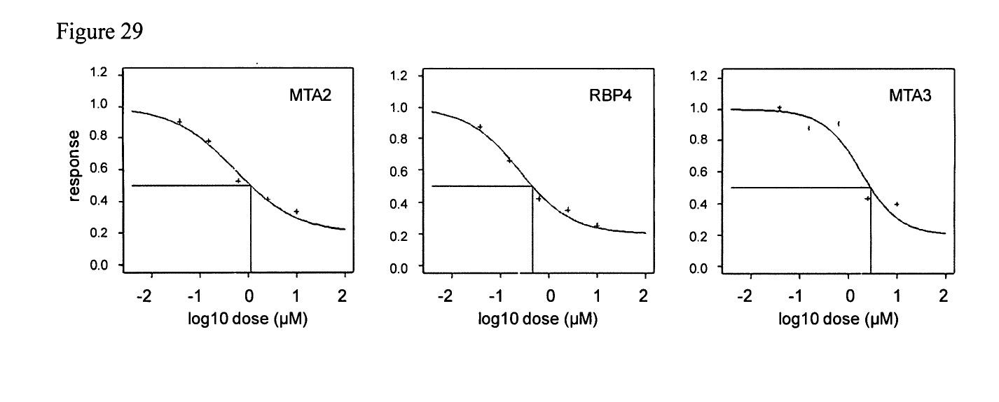

Examples of dose response curves for individual proteins are shown in FIGS. 27 to 29.

TABLE 9
Selectivity profile for SAHA (Jurkat, Ramos cells; experiment X003475)

| Representative | Protein | Protein | Protein | Redundant | Quantified | IC₅₀ |
|---|---|---|---|---|---|---|
| Sequence | Name | Complex | Function | Peptides | Spectra | (μM) |
| IPI00013774.1 | HDAC1 | CLASS I HDAC | HDAC | 52 | 25 | 0.78 |
| IPI00289601.10 | HDAC2 | CLASS I HDAC | HDAC | 42 | 15 | 2.81 |
| IPI00005711.4 | HDAC6 | CLASS II HDAC | HDAC | 24 | 22 | 0.20 |
| IPI00217540.7 | AOF2 | CoREST | HDAC | 82 | 34 | 0.14 |
| | | | Interactor | | | |
| IPI00008531.1 | RCOR1 | CoREST | HDAC | 12 | 8 | 0.16 |
| | | | Interactor | | | |
| IPI00914887.1 | RCOR3 | CoREST | HDAC | 16 | 4 | 0.15 |
| | | | Interactor | | | |
| IPI00171798.1 | MTA2 | Mi-2/NuRD | HDAC | 20 | 13 | 0.53 |
| | | | Interactor | | | |
| IPI00165357.4 | MTA3 | Mi-2/NuRD | HDAC | 10 | 4 | 1.82 |
| | | | Interactor | | | |
| IPI00216047.3 | SMARCC2 | SMRT/NCoR | HDAC | 26 | 6 | 10.00 |
| | | | Interactor | | | |
| IPI00002922.5 | TBL1XR1 | SMRT/NCoR | HDAC | 12 | 10 | 0.22 |
| | | | Interactor | | | |
| IPI00328319.8 | RBBP4 | Sin3/Mi2-NuRD | HDAC | 34 | 6 | 0.24 |
| | | | Interactor | | | |
| IPI00006663.1 | ALDH2 | | | 52 | 23 | 4.81 |
| IPI00022305.4 | BZW2 | | | 8 | 7 | 10.00 |
| IPI00784154.1 | HSPD1 | | | 130 | 58 | 10.00 |
| IPI00304082.8 | ISOC1 | | | 76 | 29 | 2.19 |
| IPI00003031.3 | ISOC2 | | | 25 | 19 | 0.22 |
| IPI00012833.1 | PPP4C | | | 12 | 7 | 10.00 |
| IPI00100933.1 | PTER | | | 9 | 8 | 10.00 |

### Example 6

### Comparison of IC₅₀ Inhibition Values for Trichostatin A with Literature Data

This examples shows a comparison of IC₅₀ inhibition values for the HDAC inhibitor Trichostatin A, a hydroxamic acid, as determined by the present invention using endogenous HDACs (example 3: Molt4 cell lysate; example 4: K-562 cell lysate) with inhibition values reported in the literature which were generated in conventional in vitro HDAC enzyme assays (Blackwell et al., 2008. Life Sciences 82(21-22):1050-1058; Khan et al., 2008. Biochemical Journal 409(2):581-589).

Blackwell and colleagues used HDAC isoforms expressed as 6×His-tagged fusion proteins in a baculovirus system in Sf9 insect cells (HDACs 1, 2, 3, 6 and 8 were expressed as full length fusion proteins; the HDAC10 fusion protein was expressed as a carboxy-terminal deletion of 38 amino acids, residues 632-669) and amino terminal carboxyfluorescein (FAM) labelled acetylated peptides as enzyme substrates (Blackwell et al., 2008. Life Sciences 82(21-22):1050-1058).

Khan and colleagues used GST-tagged or FLAG-epitope tagged recombinant human HDACs in insect Sf9 cells with a baculoviral expression system and a Fluor de Lys™ (Biomol International) HDAC assay (Khan et al., 2008. Biochemical Journal 409(2):581-589).

TABLE 10
Comparison of IC₅₀ values for Trichostatin A.
IC₅₀ values that could not be derived (as
maximal inhibition could not be achieved) are
represented as greater than the highest
concentration used in the assay. Conversely, if
maximal inhibition was achieved at the
lowest concentration the value is designated less
than the lowest concentration measured.
HDACs in which IC₅₀ values were not obtained
are designate “ND” for not determined.

| | | IC₅₀ (nM) | IC₅₀ (nM) | IC₅₀ (nM) | IC₅₀ (nM) |
|---|---|---|---|---|---|
| | HDAC | Molt4 | K-562 | Blackwell | Khan et al., |
| HDAC | class | Example 3 | Example 4 | et al., 2008 | 2008 |
| HDAC1 | I | 40 | 34 | 5 | 2 |
| HDAC2 | I | 50 | 45 | 21 | 3 |
| HDAC3 | I | 90 | 125 | <5 | 4 |
| HDAC6 | IIb | 400 | 635 | <5 | 3 |
| HDAC8 | I | >3000 | >3000 | 1100 | 456 |
| HDAC10 | IIb | >3000 | >3000 | 5 | ND |

The first observation is that IC₅₀ values as determined in the present invention are generally higher (less potent) than the values obtained with recombinant HDAC enzymes. Nonetheless, values are in good agreement for the two independent experiments performed in Molt 4 and K-562 cell lysates.

The second observation is that different selectivity profiles for Trichostatin A are obtained by the two approaches. For example, Blackwell et al. and Khan et al. report for HDAC6 (class IIb HDAC) very potent inhibition similar to HDAC 1 (class I HDAC) whereas in the present method HDAC6 is tenfold (Molt4) or eighteenfold (K-562) less sensitive to inhibition with Trichostatin A than HDAC1. In addition, Blackwell reports an IC₅₀ value of 5 nM for HDAC 10 (class IIb HDAC) whereas no significant inhibition was measured in Example 3 or 4 for HDAC10.

### Example 7

### HDAC Experiment Using an Immobilized Compound and a K-562 Cell Lysate with HDAC Inhibitor MS-275

This example illustrates the use of a competition binding assay in cell lysate to identify and characterize HDAC protein complexes and to establish the selectivity profile of the HDAC inhibitor MS-275 (Suzuki et al., 1999. J. Med. Chem. 1999; U.S. Pat. No. 6,174,905). This compound was added at defined concentrations (300 μM, 75 μM, 18.75 μM, 4.69 μM and 1.18 μM) to aliquots of K-562 cell lysates thereby allowing the MS-275 compound to bind to the target proteins in the lysate. Then the lysate was contacted with the immobilized compound (FIG. 2) to capture remaining free target proteins. The proteins bound to the immobilized compound were eluted with detergent-containing buffer, separated on a SDS-polyacryamide gel and analyzed by mass spectrometry as described in example 2.

TABLE 11
Selectivity profile for MS-275 (K-562; experiment X010500)

| Representative | Protein | Protein | Protein | Redundant | Quantified | IC₅₀ |
|---|---|---|---|---|---|---|
| Sequence | Name | Complex | Function | Peptides | Spectra | (μM) |
| IPI00013774.1 | HDAC1 | CLASS I HDAC | HDAC | 286 | 77 | 300.00 |
| IPI00289601.10 | HDAC2 | CLASS I HDAC | HDAC | 334 | 105 | 300.00 |
| IPI00217965.1 | HDAC3 | CLASS I HDAC | HDAC | 117 | 36 | 72.79 |
| IPI00747259.2 | HDAC8 | CLASS I HDAC | HDAC | 27 | 9 | 300.00 |
| IPI00012439.5 | HDAC10 | CLASS II HDAC | HDAC | 126 | 20 | 300.00 |
| IPI00005711.4 | HDAC6 | CLASS II HDAC | HDAC | 140 | 129 | 300.00 |
| IPI00217540.7 | AOF2 | CoREST | HDAC Interactor | 568 | 256 | 300.00 |
| IPI00293963.4 | CDYL | CoREST | HDAC Interactor | 20 | 4 | 300.00 |
| IPI00215963.5 | GSE1 | CoREST | HDAC Interactor | 144 | 125 | 300.00 |
| IPI00018924.3 | HMG20A | CoREST | HDAC Interactor | 65 | 46 | 300.00 |
| IPI00464951.5 | HMG20B | CoREST | HDAC Interactor | 32 | 25 | 300.00 |
| IPI00008531.1 | RCOR1 | CoREST | HDAC Interactor | 117 | 97 | 300.00 |
| IPI00914887.1 | RCOR3 | CoREST | HDAC Interactor | 186 | 73 | 300.00 |
| IPI00478128.2 | GATAD2A | Mi-2/NuRD | HDAC Interactor | 54 | 16 | 300.00 |
| IPI00103554.1 | GATAD2B | Mi-2/NuRD | HDAC Interactor | 26 | 16 | 300.00 |
| IPI00439194.1 | MBD3 | Mi-2/NuRD | HDAC Interactor | 78 | 35 | 300.00 |
| IPI00171798.1 | MTA2 | Mi-2/NuRD | HDAC Interactor | 97 | 85 | 300.00 |
| IPI00165357.4 | MTA3 | Mi-2/NuRD | HDAC Interactor | 70 | 18 | 300.00 |
| IPI00012301.1 | GPS2 | SMRT/NCoR | HDAC Interactor | 14 | 7 | 219.49 |
| IPI00216047.3 | SMARCC2 | SMRT/NCoR | HDAC Interactor | 20 | 6 | 300.00 |
| IPI00640917.1 | TBL1X | SMRT/NCoR | HDAC Interactor | 74 | 10 | 78.00 |
| IPI00002922.5 | TBL1XR1 | SMRT/NCoR | HDAC Interactor | 60 | 30 | 60.14 |
| IPI00022019.1 | SAP30 | Sin3 | HDAC Interactor | 11 | 11 | 300.00 |
| IPI00170596.1 | SIN3A | Sin3/CoREST | HDAC Interactor | 29 | 24 | 300.00 |
| IPI00328319.8 | RBBP4 | Sin3/Mi2-NuRD | HDAC Interactor | 114 | 26 | 300.00 |
| IPI00646512.1 | RBBP7 | Sin3/Mi2-NuRD | HDAC Interactor | 141 | 14 | 300.00 |
| IPI00006663.1 | ALDH2 | | | 296 | 264 | 300.00 |
| IPI00872929.2 | APOBEC3C | | | 46 | 21 | 300.00 |
| IPI00022305.4 | BZW2 | | | 120 | 50 | 300.00 |
| IPI00057097.3 | DNTTIP1 | | | 21 | 18 | 300.00 |
| IPI00784154.1 | HSPD1 | | | 190 | 175 | 300.00 |
| IPI00304082.8 | ISOC1 | | | 325 | 280 | 300.00 |
| IPI00003031.3 | ISOC2 | | | 424 | 359 | 300.00 |
| IPI00006408.4 | NOSIP | | | 7 | 7 | 300.00 |
| IPI00012833.1 | PPP4C | | | 36 | 26 | 300.00 |
| IPI00100933.1 | PTER | | | 126 | 61 | 300.00 |
| IPI00005492.2 | WDR5 | | | 30 | 25 | 300.00 |

### Example 8

### Comparison of IC₅₀ Inhibition Values for MS-275 with Literature Data

This example shows a comparison of IC₅₀ inhibition values for the HDAC inhibitor MS-275, a benzamide compound (Suzuki et al., 1999. J. Med. Chem. 1999; U.S. Pat. No. 6,174,905), as determined by the present invention using endogenous HDACs (example 7: K-562 cell lysate) with inhibition values reported in the literature which were generated in conventional in vitro HDAC enzyme assays (Blackwell et al., 2008. Life Sciences 82(21-22):1050-1058; Khan et al., 2008. Biochemical Journal 409(2):581-589).

Blackwell and colleagues used HDAC isoforms expressed as 6× His-tagged fusion proteins in a baculovirus system in Sf9 insect cells (HDACs 1, 2, 3, 6 and 8 were expressed as full length fusion proteins; the HDAC10 fusion protein was expressed as a carboxy-terminal deletion of 38 amino acids, residues 632-669) and amino terminal carboxyfluorescein (FAM) labelled acetylated peptides as enzyme substrates (Blackwell et al., 2008. Life Sciences 82(21-22):1050-1058).

Khan and colleagues used GST-tagged or FLAG-epitope tagged recombinant human HDACs in insect Sf9 cells with a baculoviral expression system and a Fluor de Lys™ (Biomol International) HDAC assay (Khan et al., 2008. Biochemical Journal 409(2):581-589).

TABLE 12
Comparison of IC₅₀ values for MS-275.
IC₅₀ values that could not be derived (as
maximal inhibition could not be achieved)
are represented as greater than the highest
concentration used in the assay. Conversely, if
maximal inhibition was achieved at the
lowest concentration the value is designated
less than the lowest concentration measured.
HDACs in which IC₅₀ values were not obtained
are designate “ND” for not determined.

| | | IC₅₀ (μM) | IC₅₀ (μM) | IC₅₀ (μM) |
|---|---|---|---|---|
| | HDAC | K-562 | Blackwell | Khan et al., |
| HDAC | class | Example 7 | et al., 2008 | 2008 |
| HDAC1 | I | >300 | 13 | 0.181 |
| HDAC2 | I | >300 | 0.51 | 1.155 |
| HDAC3 | I | 73 | 0.07 | 2.311 |
| HDAC6 | IIb | >300 | 21 | >10 |
| HDAC8 | I | >300 | >30 | >10 |
| HDAC10 | IIb | >300 | 11.5 | ND |

In the present method MS-275 inhibits of the six detected HDACs only HDAC3 with an IC₅₀ value of 73 μM. Blackwell et al. observe a less selective profile with HDAC3 as most sensitive to MS-275 inhibition followed by HDAC2, 10, 1 and 8. Khan et al. find that MS-275 is most potent for HDAC1 followed by HDAC2 and HDAC3.

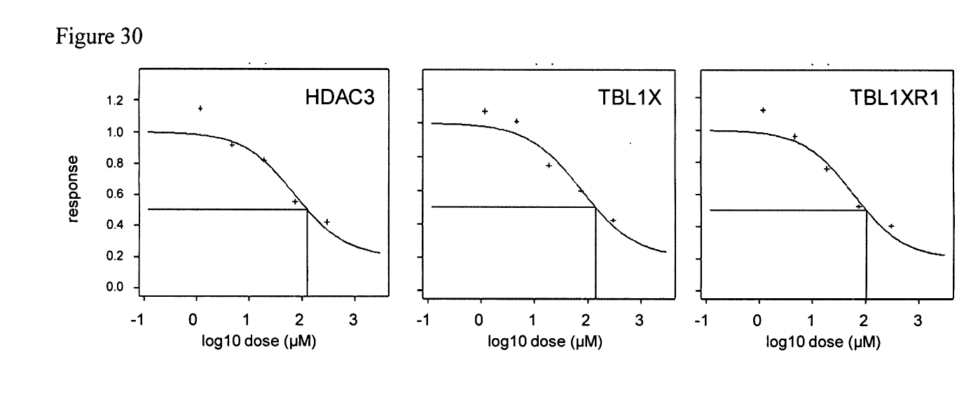

The finding of the present study that MS-275 selectively inhibits HDAC3 is further supported by the inhibition values for the NCoR complex components TBL1X (IC₅₀=78 μM), TBL1XR1 (IC₅₀=60 μM), and GPS2 (IC₅₀=219 μM) (Table 11 and FIG. 30). No other HDAC complex components detected in this experiment are affected by MS-275 (Table 11). It is known that HDAC3 forms the core of the NCoR/SMRT complex (Yang and Seto, 2008. Nat. Rev. Mol. Cell. Biol. 9(3):206-218, see Table 1).

### Example 9

### Chemoproteomics Profiling of HDAC Inhibitors Reveals Targeting of Multiple HDAC Complexes with Compound Class-Dependent Selectivity

Histone deacetylase inhibitors (HDACi) display marked anti-cancer and anti-inflammatory properties but the development of isoform-selective drugs has proven challenging.

This example shows the chemoproteomics profiling of 16 HDACi using a hydroxamate affinity matrix and quantitative mass spectrometry reveals that these drugs target multiple HDAC complexes built around ELM-SANT domain scaffolds, including a novel mitotic deacetylase complex (MiDAC). The targeted complexes were further characterized by quantitative immunoaffinity purifications. Inhibitors cluster in distinct groups by their target profiles with aminobenzamides showing preferential binding to the HDAC3/NCoR complex, and several non-HDAC targets for the hydroxamate class were identified. Distinct inhibitor target profiles correlate with differential effects on downstream targets. Application of the approach to compound screening identified the anti-inflammatory drug bufexamac as a Class IIb (HDAC6; HDAC10) inhibitor. Our approach enables the discovery of both novel targets and inhibitors and suggests that compound selectivity should be considered in the context of protein complexes.

Protein lysine acetylation is a key mechanism in the epigenetic control of gene expression and the regulation of cell metabolism¹⁻³, and the partaking enzymes represent targets for the therapy of cancer, autoimmunity, and neurodegenerative disease⁴. The first mammalian histone deacetylase was discovered in 1996 by means of a chemical biology approach⁵. Based on sequence phylogeny and function, there are four distinct classes: Class I (HDAC1, 2, 3, and 8), class IIa (HDAC4, 5, 7, and 9), class IIb (HDAC6 and 10) and class IV (HDAC11) represent Zn²⁺-dependent amidohydrolases, whereas class III comprises the mechanistically diverse NAD⁺-dependent sirtuins⁶. HDACs form the catalytic core of megadalton complexes involved in chromatin modification and gene repression. Four such molecular machines have been characterized to date: The CoREST, NuRD, and Sin3 complexes contain a HDAC1/HDAC2 dimer as core, whereas the NCoR complex is formed around HDAC3⁷. The roles of these complexes are diverse and often cell-type specific, and whereas more data are emerging regarding their role in the determination of cell fate, their functions in tissue homeostasis are less well understood⁸. The CoREST complex couples histone deacetylation to demethylation to repress neuronal genes^{9, 10}, the NuRD complex links deacetylation to a chromatin-remodeling ATPase and promotes gene silencing^{11, 12}, and the Sin3 complex represses genes downstream of various developmental pathways^{13, 14}. The NCoR complex is the major corepressor for nuclear receptors^{15, 16}. Class IIa HDACs exhibit low enzymatic activity and were proposed to have mainly “modification reader” or other scaffold functions^{17, 18}. Class IIb HDACs are thought to have mostly non-epigenetic functions in regulating protein folding and turnover¹⁹.

Small molecule HDAC inhibitors (HDACi) were discovered by their ability to induce redifferentiation of transformed cells²⁰. SAHA (vorinostat) and romidepsin are approved for the treatment of cutaneous T cell lymphoma, valproate is in clinical use as an anticonvulsant, and several HDACi are in clinical development for a number of indications²¹. The development of HDACi has been hampered by a lack of target selectivity which increases the risk of toxic liabilities and also limits their use as tool compounds²¹. The perceived lack of selectivity may originate from the optimization of these compounds in industry standard assays using recombinant enzymes or protein fragments, which do not properly reflect the native conformation and activity of the target and its physiological context, due to incorrect protein folding, post-translational modifications, and absence of regulatory subunits. Notably, purified class I HDACs exhibit increased activity in the presence of interacting proteins^{22, 23}. Most HDACi adhere to a distinctive pharmacophore comprising a “cap”, which binds to the rim of the substrate channel, a spacer spanning the channel, and a Zn²⁺-chelating function. A photoaffinity analog of SAHA was shown to label not only HDACs but also RCOR1, MBD3 and MTA1/2, indicating that these proteins are in proximity of the active site, and purporting that the “cap” conveys inhibitor selectivity²⁴.

Recent advances in quantitative chemoproteomics have enabled binding studies of small molecule enzyme inhibitors to endogenous proteins in cells and tissues²⁵⁻²⁷. Here we extend this approach towards the monitoring of clinical and tool HDACi binding to native megadalton protein complexes, and towards the discovery of novel targets and inhibitors. We find that HDACi target known and novel protein complexes which are precisely defined by matching IC₅₀ values for a given inhibitor for all complex subunits, and confirm complex composition by quantitative immunoaffinity purifications. Notably, inhibitor selectivity data for native drug target complexes deviate from literature values obtained with recombinant enzymes in isolation, indicating in some cases an unexpected degree of selectivity.

### Results

### Synthesis of a Target Class Specific HDAC Probe Matrix

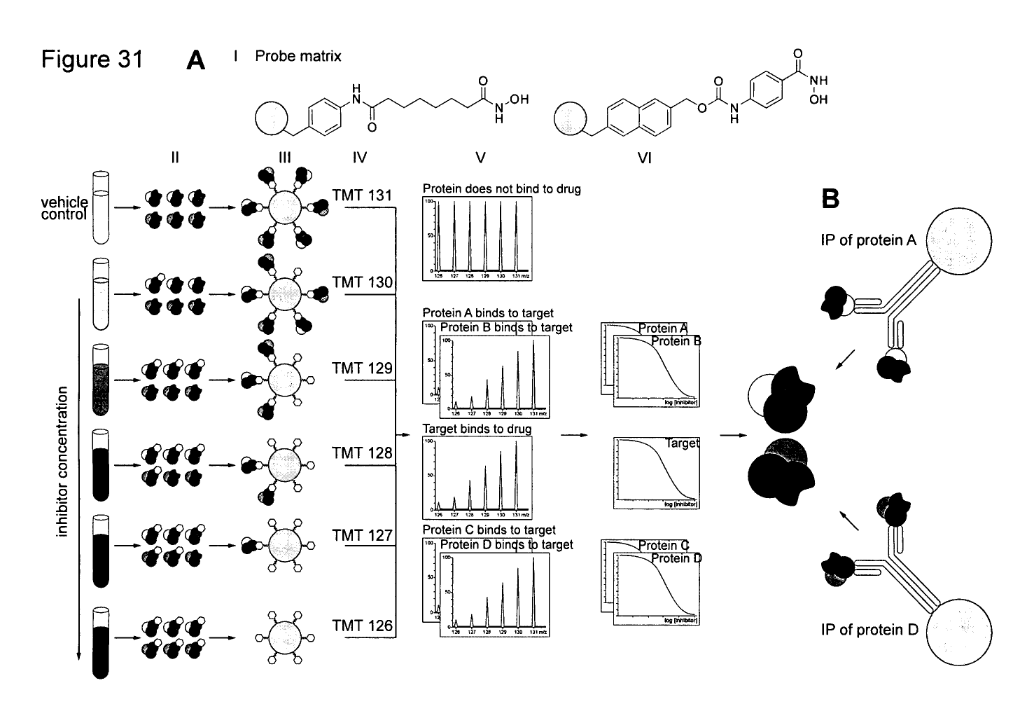

Target-class directed chemical probes provide tools for the identification of drug targets directly in cells and tissues. Suitable probes consist of a moiety which binds to a ligand pocket conserved within the target class under investigation, and a functional group for immobilization, thus enabling efficient enrichment of bound proteins for analysis^{28, 29}. HDACs share a conserved substrate pocket, and most hydroxamate inhibitors are nonselective³⁰. We synthesized a target-class specific probe matrix by derivatizing sepharose with analogues of the hydroxamates SAHA and ITF2357 (givinostat). The probe matrix was exposed to cell extracts under close to physiological conditions and aliquots of the sample were treated with excess inhibitor which competes with the immobilized probes for target protein binding. The difference in captured proteins between vehicle-treated and inhibitor-treated samples was quantified by isobaric tagging (iTRAQ or TMT) of tryptic peptides and tandem mass spectrometry analysis (MS/MS) of the combined peptide pools³¹. For each identified protein the decrease of reporter signal responses relative to the vehicle control reflects the competition by the ‘free’ inhibitor drug for its target. The results comprise binding data for both direct enzyme targets as well as proteins residing in a complex with the target, since these are predicted to exhibit matching IC₅₀ profiles across a set of drugs. Associations of proteins in complexes were confirmed by immunoaffinity purifications (FIG. 31).

### Quantitative Mapping of Probe Matrix Interactomes

Initially we tested the binding of the probe matrix to recombinant HDACs 1-11 purified from Sf9 cells. The proteins were found to bind to the matrix, but the amount captured was only affected at a high excess of free SAHA, indicating that the bulk of the purified enzyme exhibit low activity. The activity of the enzymes in peptide deacetylation assays may be due to a small fraction of properly folded protein, or due to contamination with co-purifying insect cell activities.

More substantial results were obtained when the SAHA- or ITF2357-derivatized matrix was used to probe endogenous HDACs in K562 whole cell extracts. From the approx. 2900 proteins captured and quantified by MS/MS, about 300 exhibited competition by a large excess of inhibitor and thus were considered specific, whereas the remainder was considered background. All class I/class IIb HDACs and many known HDAC complex subunits were captured in specific fashion, as binding was competed by excess inhibitor. In addition many other proteins were specifically captured, implicating them either as novel inhibitor targets or as co-purifying proteins in target complexes. Compared to the SAHA matrix, the ITF2357 matrix exhibited higher background but specifically retained a few additional proteins. Since both types of matrix captured the same set of HDACs, other proteins found primarily with one type of matrix represent drug-specific off-targets or interactors thereof. Examples are 5,10-methenyltetrahydrofolate synthetase (MTHFS) and hydroxysteroid (17-beta) dehydrogenase 4 (HSD17B4), which emerge as prominent givinostat but not SAHA off-targets. We defined the specific set of captured proteins as the probe matrix interactome. Taking the results from several experiments together, the matrix interactome comprises approx. 500 proteins including the six class I and class IIb HDACs, and 29 proteins known to associate with class I HDACs in the CoREST, NuRD, Sin3, and NCoR complexes. The remaining proteins without a previous connotation to deacetylases are likely to represent either off-targets or proteins interacting with HDACs or with off-targets. Next, the probe matrix interactome was differentially mapped in nuclear versus cytosolic fractions of Jurkat cells, and in whole cell extracts from six human cell lines and six mouse tissues. Most proteins in the interactome appeared to be ubiquitously expressed, and as expected the majority of proteins are enriched in the nuclear fraction as compared to whole cell extract. Because of the function of class I HDACs in cell division³², we also conducted differential mapping of the interactome in HeLa cells arrested in mitosis (by nocodazole treatment) compared to G1/S phase (by aphidicolin treatment) and untreated cells. Notably three proteins were captured in increased amounts from mitotic cells and may constitute a novel HDAC complex; DNTTIP1 (deoxynucleotidyltransferase interacting protein), C14ORF43, a protein of unknown function with homology to the REST corepressor which we dubbed MIDEAS (for mitotic deacetylase-associated ELM and SANT domain), and the histone acetyltransferase CDYL. Remarkably, the expression level of DNTTIP1 is not increased in mitotic cells, and hence the data indicate the formation of a dedicated mitotic complex.

### Proteomic Target Profiling of Clinical and Tool Compounds

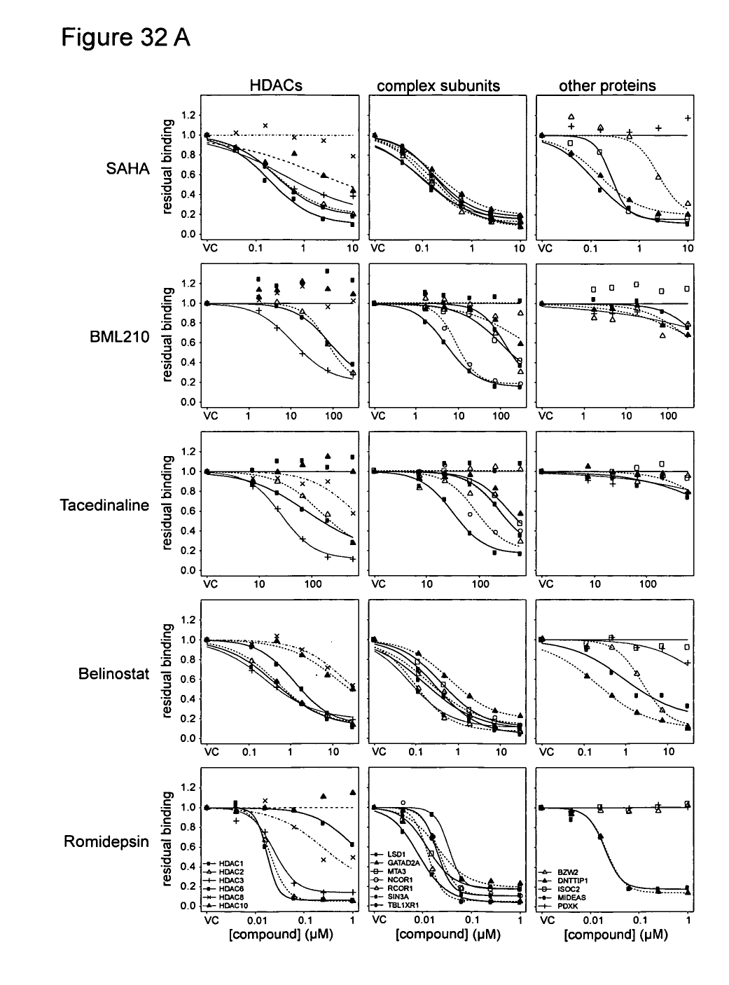

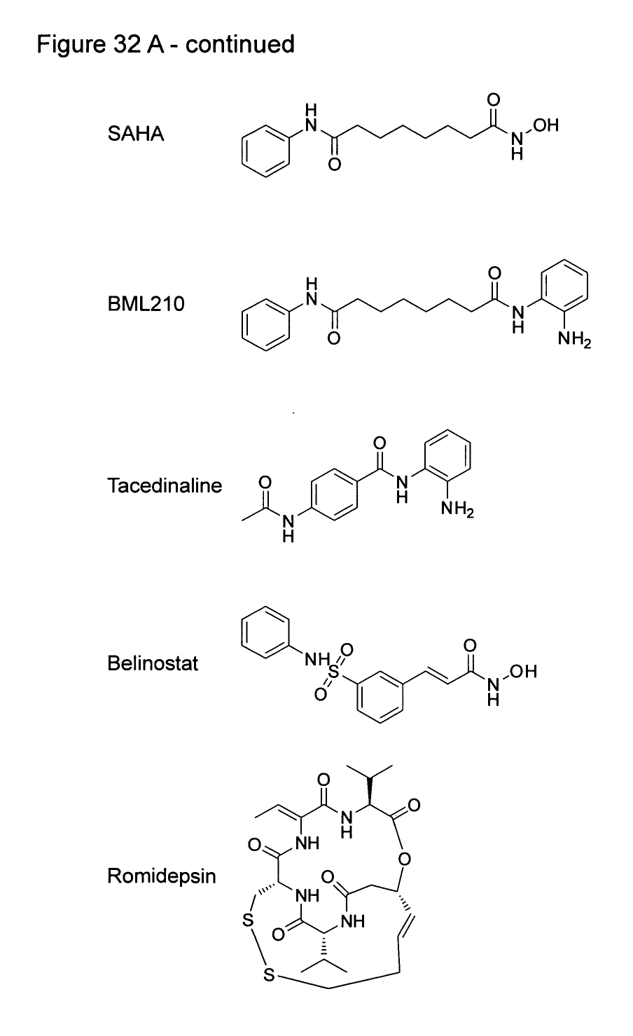

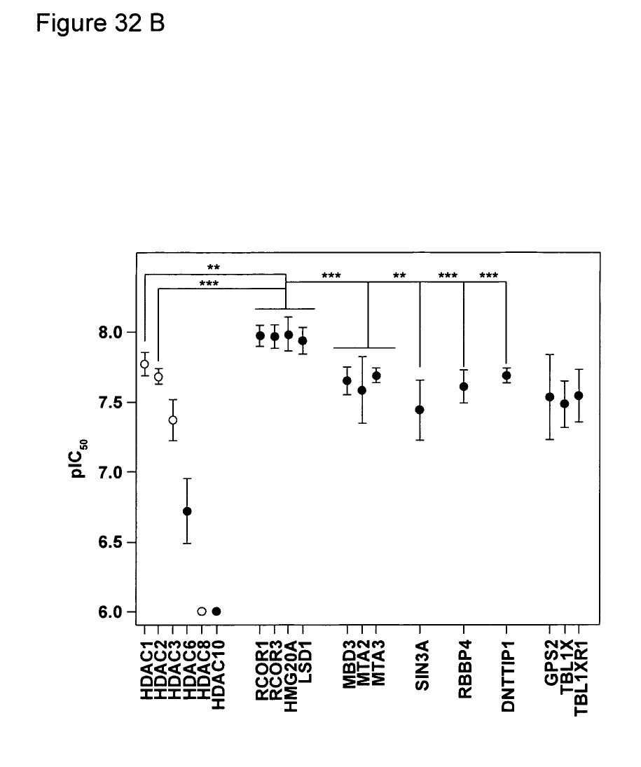

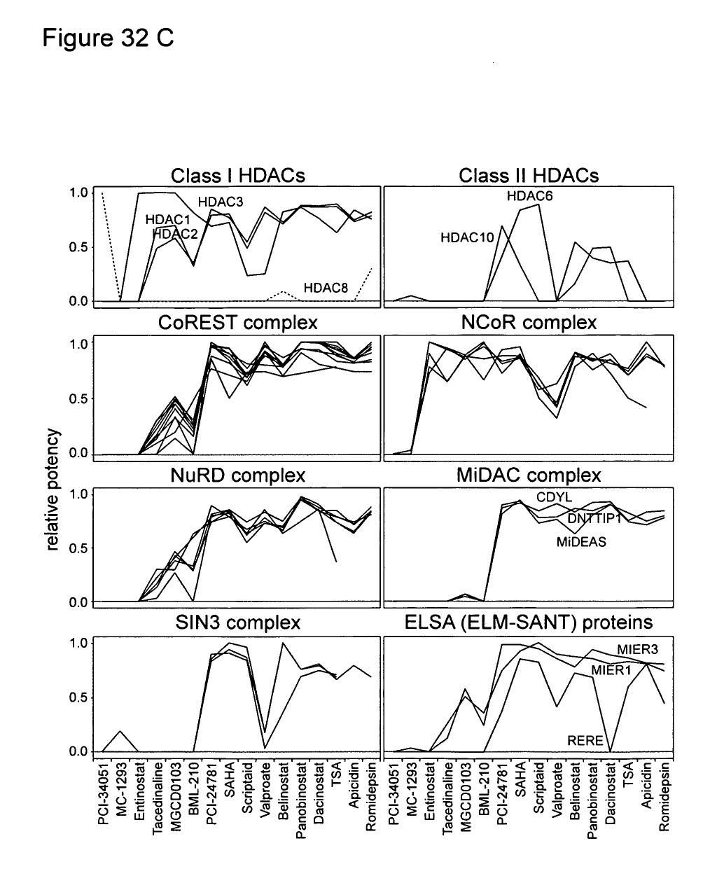

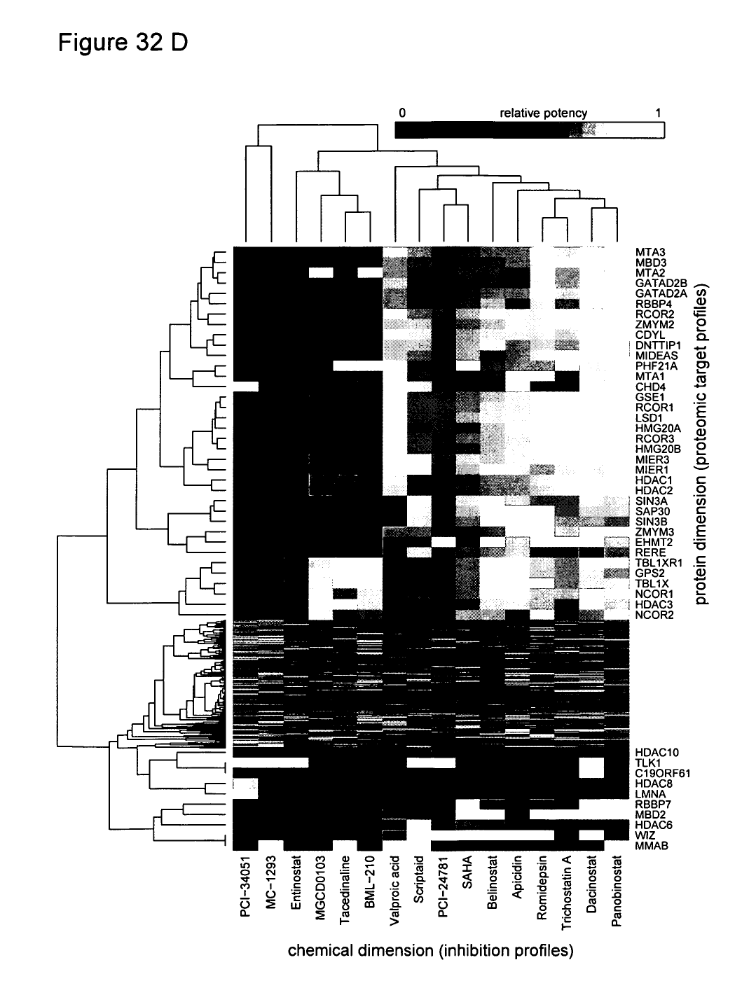

The probe matrix offers an efficient tool to probe a subproteome of putative targets with drugs under close to physiological conditions. We selected a set of 16 structurally diverse HDACi including approved drugs, clinical development compounds, and tool compounds. Aliquots of K562 cell extract were spiked with vehicle or different inhibitors in 5 concentrations typically ranging from 40 nM to 10 μM. For low or very high potency compounds, concentrations were adjusted. Subsequently, samples were incubated with the probe matrix, captured proteins were quantitatively mapped by MS/MS, and a set of IC₅₀ values was determined for each inhibitor (FIG. 32a). To assess the reproducibility of our methodology, two to seven replicate experiments were performed per inhibitor profile using targeted data acquisition³³ for a defined subset of proteins. The data were sufficiently reproducible to distinguish fairly small (two-fold) IC₅₀ differences and we found small albeit statistically significant potency differences between distinct HDAC1/2 complexes, e.g. NuRD and CoREST (FIG. 32b). To determine the impact of target protein depletion by the probe matrix on the IC₅₀ values, an aliquot of the cell extract was subjected to two sequential incubations with the matrix. This procedure allows the calculation of apparent dissociation constants (K_{d}^{app})²⁷. However the resulting deviation between K_{d}^{app} and IC₅₀ values was less than twofold for 99% of the proteins.

The dataset comprises concentration-inhibition profiles for 16 compounds versus a total of 344 proteins, including the class I and IIb HDACs, components of HDAC complexes, and putative novel complex components or targets (Table 13). Proteins associated in a complex exhibit matching K_{d}^{app} values for a given inhibitor, implying that these proteins remain associated with the molecular target during the assay procedure. Thus, the inhibition profiles across the compound set can be used to trace protein complexes. It should be noted that proteins known to reside in two or more complexes (e.g. HDAC1, HDAC2, RBBP4, RBBP7 and LSD 1^{7,34}) are likely to exhibit K_{d}^{app} values representing aggregates between complexes. While the HDAC1/2-containing CoREST and NuRD complexes showed a similar inhibition profile across the compound set, there were marked differences in the Sin3A inhibition profile, with all benzamides showing a pronounced drop in potency for this complex compared to CoREST and NuRD. The profiles accurately delineate a novel mitotic deacetylase complex (termed MiDAC) formed by HDAC1/2, DNTTIP1, CDYL and MIDEAS (FIG. 32c). Bidirectional hierarchical clustering of the complete dataset clearly outlined the major complexes including MiDAC (FIG. 32d). In the chemical dimension the clustering is driven by the major chemotypes with several hydroxamate sub-clusters which differed in their effect on Class II HDACs. In agreement with published data we found that the peptidic and hydroxamate compounds are substantially more potent than the aminobenzamides (Table 13). A notable observation is the unexpected degree of selectivity of benzamide class inhibitors which show a preference for the HDAC3-NCoR complex. It is interesting to compare the target profiles of SAHA and its analog BML-210, which are identical except an amidobenzamide group in BML-210 replacing the hydroxamate function, causing a general drop of potency for class I HDACs (and complete loss of potency for class IIb)) concomitant with an increase in selectivity for HDAC3 over HDAC1/HDAC2 (FIG. 32a).

### Deconvolution of Protein Complexes by Quantitative IP-MS/MS

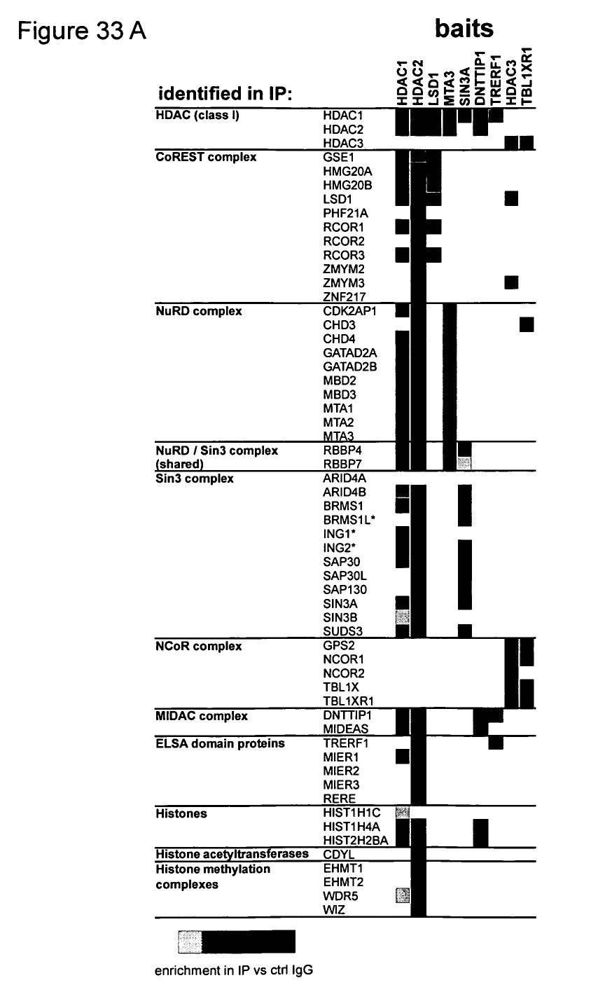

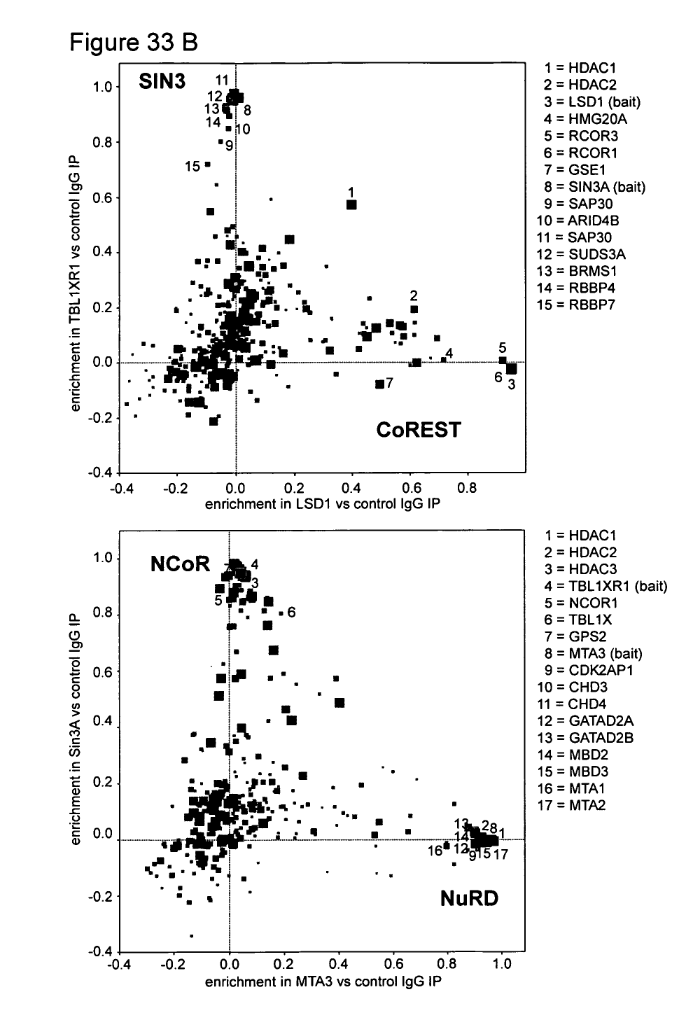

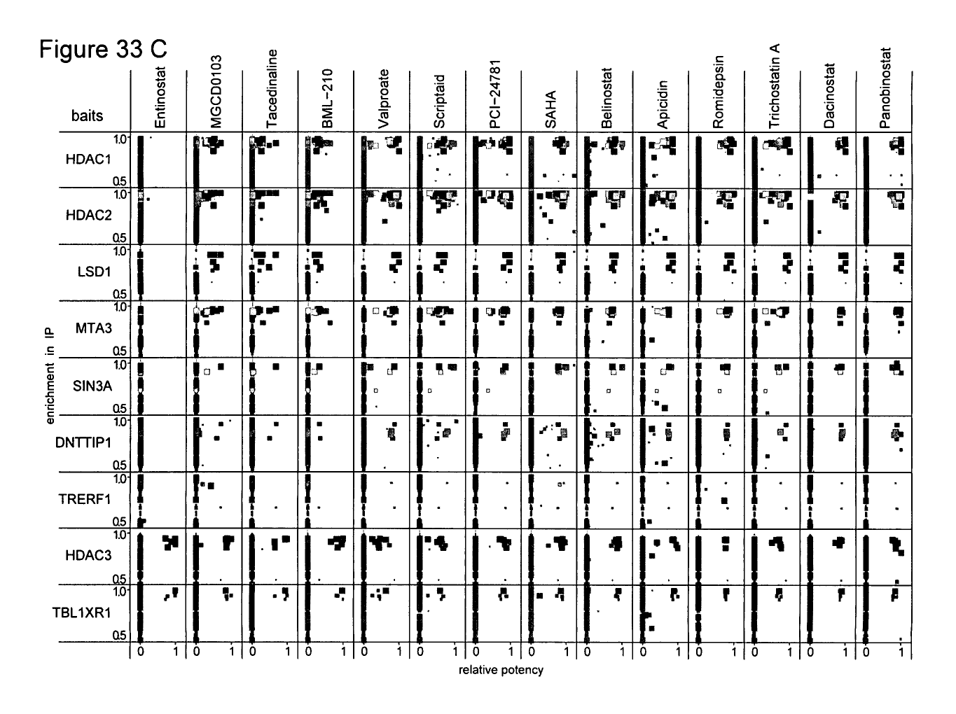

In order to differentiate between novel direct targets and novel components of HDAC complexes, a set of quantitative co-immunoaffinity purifications (co-IP) with quantitative MS/MS analysis was performed. We evaluated a set 21 antibodies and ultimately selected nine, directed against three class I HDACs, four known complex subunits (LSD1 from CoREST, MTA3 from NuRD, SIN3A from Sin3, TBL1XR1 from NCoR), and two novel complex components (TRERF1, DNTTIP1) (FIG. 33a). After isobaric tagging, IP samples from two different antibodies each were combined with matched control IgG samples for MS/MS analysis such that specifically co-immunopurified proteins were clearly discriminated from background (FIG. 33b). Most known complex components and several of the new targets or components were identified in the IP samples. However, in some cases the IP samples contained hundreds of enriched proteins interfering with the identification of true interacting proteins. Data complexity is drastically reduced when orthogonal chemoproteomics and co-IP data are combined, thus enabling efficient and unbiased deconvolution of HDACi target complexes (FIG. 33c). The MiDAC complex was confirmed in the DNTTIP1 IP sample which comprised MIDEAS, HDAC1, and HDAC2 but no known HDAC complex subunits. TRERF1, an ELSA (ELM-SANT) domain protein related to MIDEAS, was co-purified with HDAC2 and with DNTTIP1. Immunoaffinity purification of TRERF1 itself indeed confirmed its association with DNTTIP1 and HDAC1/2, but not with MIDEAS, suggesting that TRERF1 represents an alternative scaffold for a similar complex. Other ELSA proteins were also found to co-purify with HDAC1 or HDAC2 including MIER1, 2, and 3, which were identified with HDAC2, and RERE, which was identified with HDAC1 and HDAC2. Since the inhibition profiles of the MIER and RERE proteins did not align with any of the other complexes (FIG. 32c), these proteins likely represent components of distinct HDAC complexes formed around ELSA scaffolds³⁵.

### Cell-Based Profiling of Clinical and Tool Compounds

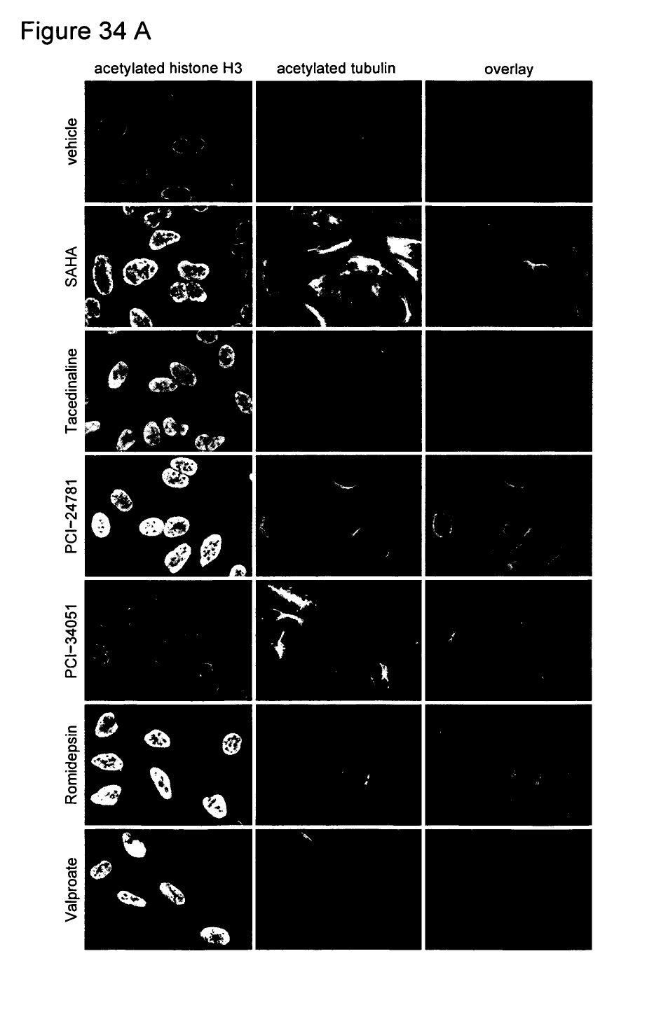

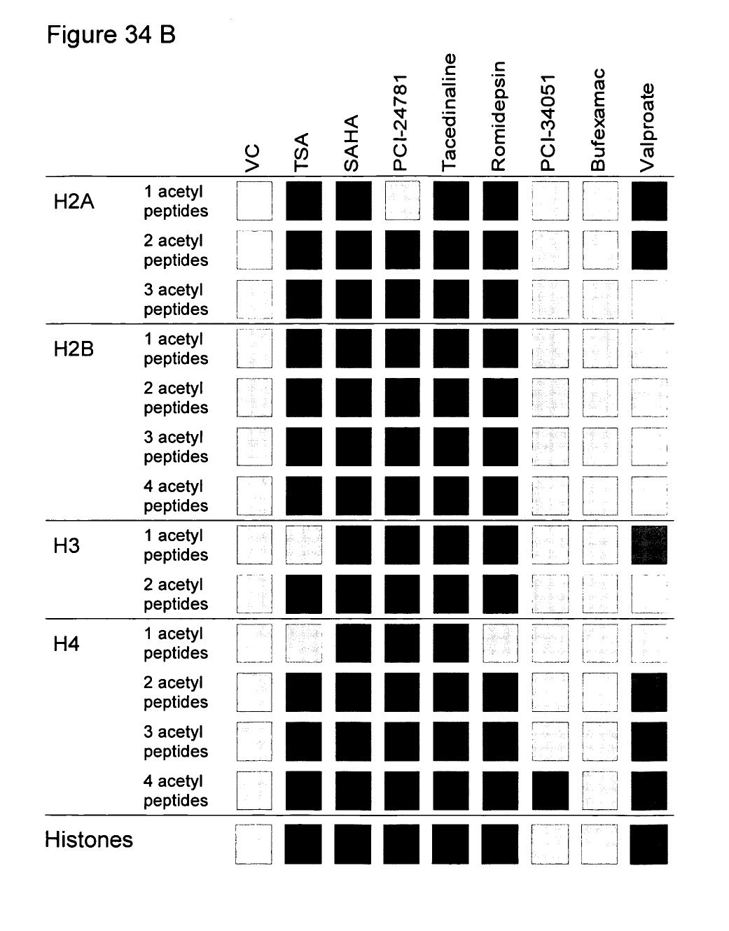

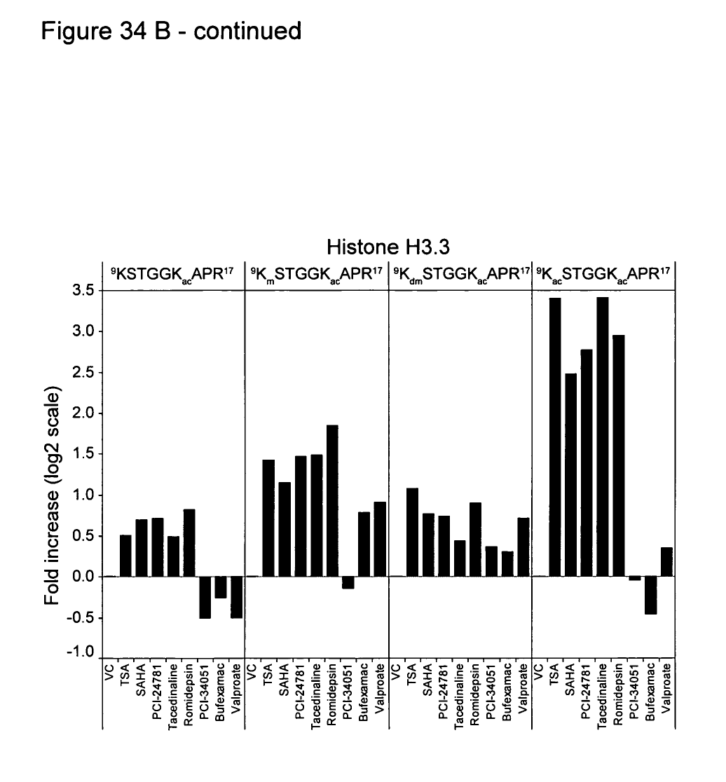

To investigate the correlation of proteomic target profiles with substrate selectivity, a subset of the reference inhibitors was subjected to cell-based tubulin and histone modification assays. K562 and HeLa cells were treated with vehicle or compounds including nonselective inhibitors (SAHA, PCI-24781), class I selective inhibitors (tacedinaline, romidepsin, valproate) and the HDAC8 inhibitor PCI-34051. Cell viability was monitored and drug effects were detected by antibodies for acetylated tubulin as the major substrate of HDAC6¹⁹ and histones H3 and H4 as the major class I HDAC substrates by immunofluorescence and western blotting (FIG. 34a). The nonselective HDACi increased steady state acetylation of tubulin and histones manifest by the staining of acetylated microtubules as well as punctuate nuclear staining of acetylated histones. The class I selective HDACi elicited staining of acetylated histones but did not affect tubulin as expected. Aliquots of vehicle-treated and drug-treated cells were also compared by differential mapping of histone acetylation and methylation marks using isobaric tagging and MS/MS (FIG. 34b). The results confirmed the range of activities observed in chemoproteomics profiling and indicate a pronounced abundance of hyperacetylated histone peptides after treatment with nonselective HDACi, in particular TSA and romidepsin. In contrast, valproate exhibited a more selective effect. For instance, the acetylation of histone H3K14 in the presence of methylated K9 was increased to a similar degree as with other nonselective HDACi, but little effect was observed on the acetylation of K9 in peptides containing acetylated K14.

### Bufexamac as a Novel Class IIb HDAC Inhibitor

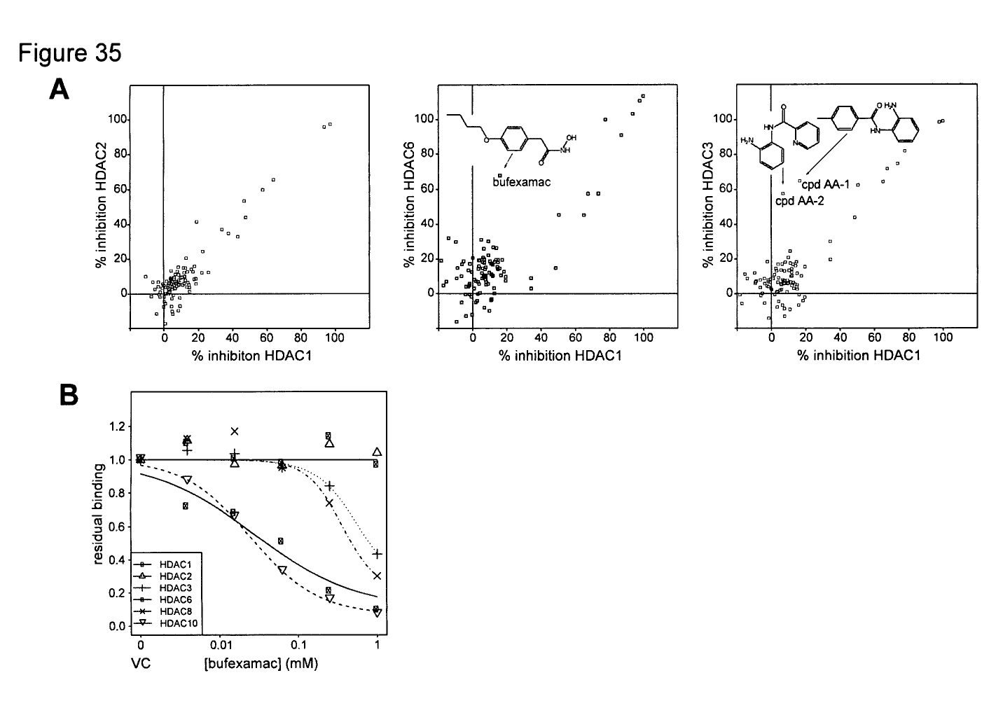

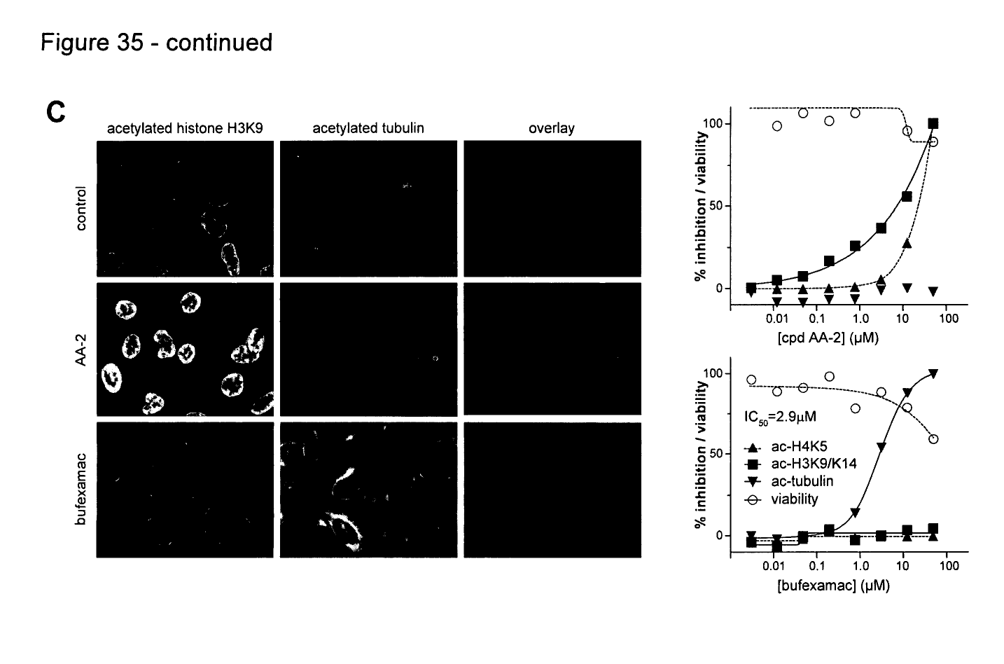

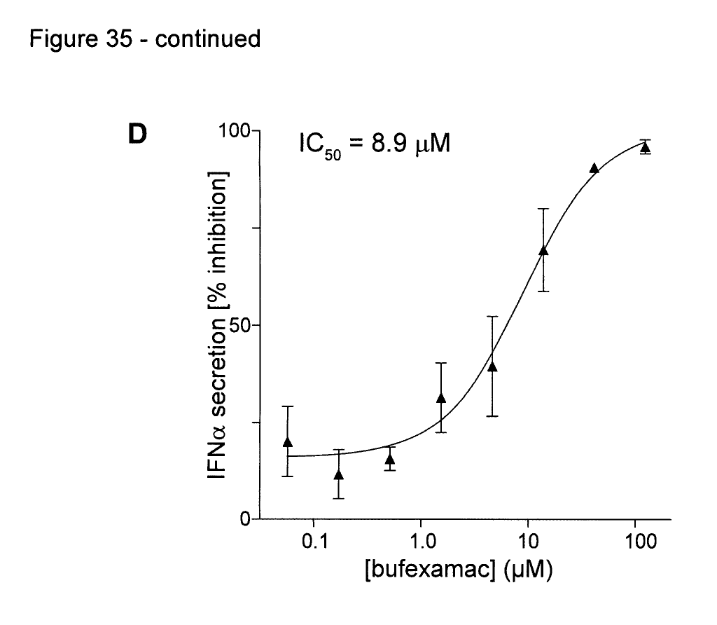

To enable the discovery of novel selective HDACi we developed a high throughput variation of the chemoproteomics protocol in which MS/MS detection is replaced with multiplexed fluorescent antibody detection on “dot blot” type arrays. The method was applied to the screening of a focused compound library in whole cell extract of Jurkat and Ramos cells for inhibitors of HDAC1, 2, 3 and 6. Several hits were obtained with a few compounds displaying a notable degree of selectivity (FIG. 35a). Two benzamides (o-aminoanilides) were identified as hit compounds exhibiting selectivity for HDAC3. Bufexamac, a non-steroidal anti-inflammatory drug with an unknown mechanism of action³⁶ was found to preferentially affect HDAC6. Bufexamac was subjected to quantitative proteomic profiling as described above for the reference HDACi set, which confirmed its selectivity for HDAC6 and HDAC10, the other class IIb isoform, in addition to several non-HDAC targets (FIG. 35b). The results are consistent with tubulin immunofluorescence and western blot data which showed a strong increase in acetylated tubulin, the major HDAC6 substrate, but not in acetylated histones as substrates of Class I HDACs (FIG. 35c, see also data in FIG. 34b). The cellular potency for tubulin deacetylation correlated with the potency for one of the drugs' anti-inflammatory effects, the secretion of IFNα in peripheral blood mononuclear cells (FIG. 35d).

### Discussion

Gene transcription and its epigenetic regulation are controlled by megadalton protein complexes^{37,38}. Hence the action of drugs which modulate epigenetic mechanisms should be considered in the context of the multiprotein complexes which constitute their targets. We developed an affinity capture method combined with highly sensitive multiplexed protein quantification by mass spectrometry to probe the interaction of drug molecules with drug targets in cells or tissue under conditions which preserve the integrity of protein complexes. The strategy, applied here to HDAC inhibitors, has the potential to be extended to other pharmacological target classes, and enables the discovery of drug leads, their molecular targets, and functional target protein complexes. The biological activity of compounds is assessed in a functional molecular context, without use of recombinant purified proteins or protein overexpression. The major prerequisite is a probe matrix which binds to a sub-proteome or interactome which is characterized by a shared chemical ligand space, typically based on a substrate- or cofactor-binding site. We developed a probe matrix which captures HDACs and related proteins by binding to the substrate pocket. The matrix interactome comprises the class I and class IIa HDACs, and the majority of previously reported subunits of HDAC complexes. Class IIa HDACs were not identified, presumably because they exhibit low catalytic activity and low affinity for the hydroxamate probes¹⁷. Moreover, the interactome contains many other enzymes which potentially represent targets sharing a similar chemical ligand space, including other metalloenzymes, as well as other proteins which may be associated with enzymes in protein complexes.

One basic application of a probe matrix is the differential expression profiling of a subproteome—its interactome—across a range of biological samples and conditions. In line with a pleiotropic function most proteins in the hydroxamate matrix interactome displayed minor differences across a panel of cell lines and tissues. However two proteins lacking an obvious small molecule binding site were captured predominantly from mitotically arrested cells, in line with the specific formation of a mitotic HDAC complex comprising novel subunits, a finding subsequently confirmed and extended by inhibitor profiling and co-IP data.

A powerful application of the probe matrix is the profiling of the interactions of drugs and lead molecules with the interactome in cell extract. We demonstrated that robust quantitative data are obtained by high sensitivity LC/LC-MS/MS to measure protein binding to the matrix in whole cell extract as a function of the concentration of competing “free” compounds. Bidirectional hierarchical clustering of the proteomic target profiles of 16 inhibitors (FIG. 32d) guides (i) the classification of drugs in selectivity clusters, which are predicted to exhibit similar pharmacological effects, (ii) the grouping of protein targets in chemical space, and (iii) the assignment of targets to protein complexes. In order to distinguish individual targets which share a structurally similar ligand binding space from proteins associated in a complex, the chemoproteomics clustering may be deconvoluted using literature data, or bioinformatics predictions of ligand binding sites³⁹. However the highest quality orthogonal data are acquired from co-IP experiments which delineate protein complexes in the same biological context, while avoiding overexpression artifacts and the impact of different cell types⁴⁰.

Our chemoproteomics dataset clusters 16 HDACi versus 344 proteins specifically interacting with the probe matrix. The clustering of inhibitors is driven by the major chemotypes represented by hydroxamates and aminobenzamides, with several hydroxamate sub-clusters, and reveals an unexpected degree of selectivity for inhibitors previously perceived as nonselective⁴¹⁻⁴³. However much of the published data is inconsistent raising issues with the enzyme assays employed. Notably, a recent carefully controlled extensive enzyme kinetic study of drugs and tool compounds also reported a higher degree of inhibitor selectivity¹⁷. We found that all compounds in the benzamide cluster showed a preference for the HDAC3-NCoR complex and that within the class I HDAC complexes they did not affect Sin3. No inhibitor outside this class displayed this profile, and this feature of the benzamide class may contribute to a more favorable toxicology profile. Remarkably, the clinically relevant drug valproate did also affect the Sin3 complex to a substantially lesser degree than NuRD and CoREST complexes.

A number of non-HDAC targets are potently affected by several hydroxamate HDACi but do not appear to be components of HDAC complexes, given that their inhibition profiles do not match any HDAC (FIG. 32c), and because they were not enriched in the co-IP set (FIG. 33a). These proteins may represent off-targets sharing a similar chemical ligand space. Examples are the basic leucine zipper/W2 domain protein BZW2 and the isochorismatase domain protein ISOC2, and several other Zn²⁺-dependent metalloenzymes including aldehyde dehydrogenases.

In the protein dimension, the clustering data delineate target protein complexes, since proteins exhibiting matching inhibition profiles across the inhibitor panel are likely to be physically associated. This is evident by the almost perfect clustering of the four major HDAC-containing complexes (FIG. 32). To our knowledge this is the first time that small molecule binding data are used to characterize target protein complexes, and hence we extended our data by an extensive co-IP analysis of endogenous HDAC complexes from the same cell extract. A few previously reported class I HDAC complex components which had been absent from the probe matrix interactome were identified in the co-IP samples and may represent interactions that are sensitive to inhibitor binding, e.g. the ING2 subunit of the Sin3 complex⁴⁴. The co-IP results confirmed the existence of the additional HDAC1/HDAC2 complexes delineated in the analysis of the chemoproteomic data. These complexes are built around ELSA domain proteins which are phylogenetically related to corepressor components of NuRD and CoREST complexes³⁵. Our data indicate that each complex contains only one type of ELSA scaffold, and that several of such complexes exist, formed around MIER1/2/3, RERE, TRERF1, and MIDEAS, a previously unannotated gene product. One function of HDAC complexes is likely the coordination of deacetylation with other epigenetic modifications. The CoREST complex couples HDACs to the demethylase LSD1¹⁰, and the ELSA proteins MIER1 and RERE were shown to scaffold HDACs with the EHMT methyltransferases³⁵, and our inhibition profiles confirmed these complexes as HDACi targets. The composition of these HDAC complexes was deconvoluted further by the co-IP data, in particular the MiDAC complex formed in mitotic cells by HDAC1/2, MIDEAS and DNTTIP1. DNTTIP1 is a DNA binding protein that has been described to modulate the activity of terminal deoxynucleotidyl transferase (TDT), a specialized DNA polymerase that incorporates nontemplated nucleotides to the 3′ end of DNA templates to mediate the junctional diversity of immunoglobulin genes⁴⁵. However, we did not consistently identify an association of TDT with the MiDAC complex suggesting a TDT independent function of the complex in cell division. The inhibition profiles also implicated the REST corepressor CDYL⁴⁶ as a component of MiDAC, in line with the increase in CDYL captured from mitotic cells by the SAHA matrix. However, CDYL co-immunopurified with HDAC2 but not with the MiDAC subunit DNTTIP1, and hence further analysis is required to clarify whether it is a component of MiDAC or of an alternative complex.

The fourth class I enzyme, HDAC8, is more difficult to assign to a complex, because it is only targeted by a few inhibitors, and we did not identify a suitable antibody to characterize it by co-IP. Notably, the HDAC8 inhibitor PCI-34051⁴⁷ is the only compound in our panel which was specific for a single HDAC, and its target profile comprised other proteins which might represent components of a complex. The class IIb enzymes HDAC6 and HDAC10 were only inhibited by hydroxamate type compounds and both do not appear to form robust complexes as no strong associations with other proteins in the inhibition profiles were detected. HDAC6 has recently been implicated in chromatin regulation⁴⁸ but the HDAC6/HDAC10 inhibitor bufexamac did not affect the acetylation of histones, which does not support a direct role of class IIb deacetylases in histone modification.

Our chemoproteomics technology can be adapted to high throughput screening by using an antibody-based readout instead of MS/MS to reduce sample requirements and process time. We conducted a screen of a focused compound library for selective inhibitors. The HDAC3 selective hit compounds were benzamides, confirming the selectivity preferences of this pharmacophore class. The screen also identified the hydroxamate bufexamac, an NSAID with an unknown mechanism of action³⁶ as a class IIb selective inhibitor. The drug induced tubulin hyperacetylation in drug concentrations matching its anti-inflammatory effect. Therefore, HDAC6 inhibition may contribute to the drug's clinical efficacy.

In conclusion, we have shown for the first time that a chemoproteomics strategy based on small molecule inhibitors can be applied to discover and classify molecular complexes around drug target proteins. The approach confirms and extends orthogonal protein-protein interaction mapping, e.g. based on co-IP methodology. We have demonstrated the utility of this strategy in drug discovery by defining distinctive target profiles for clinical HDAC inhibitors in cell extracts, and employed it in screening for novel small molecule inhibitors. The data support the value of drug discovery strategies based on target proteins in their biological context.

TABLE 13
K_{d}^{app} values (in μM) for various HDAC inhibitors for molecular targets and target complexes from chemoproteomic binding profiling
(NI: no inhibition at maximum compound concentration tested; number in italics: IC₅₀ value listed
(no K_{d}^{app} was determined); no value listed: protein was not detected in samples).

| | | | | | | Dacino- | | Mocetino- | Panobino- | | |
|---|---|---|---|---|---|---|---|---|---|---|---|
| | | | | | | stat | | stat | stat | | |
| | | | | BML- | Entinostat | (LAQ | MC- | (MGC | (LBH- | PCI- | PCI- |
| classification | protein | Apicidin | Belinostat | 210 | (MS-275) | 824) | 1293 | D0103) | 589) | 34051 | 24781 |
| class I HDAC | HDAC1 | 0.02 | 0.23 | 36 | NI | 0.01 | NI | 24 | 0.1 | NI | 0.03 |
| | HDAC2 | 0.02 | 0.28 | 35 | NI | 0.01 | NI | 39 | 0.12 | NI | 0.04 |
| CoREST | RCOR1 | 0.01 | 0.16 | 46 | NI | 0.01 | NI | 48 | 0.05 | NI | 0.02 |
| complex | RCOR2 | 0.02 | 0.14 | 85 | NI | | NI | 103 | 0.09 | NI | 0.02 |
| | RCOR3 | 0.01 | 0.15 | 55 | NI | 0.01 | NI | 60 | 0.05 | NI | 0.02 |
| | PHF21A | 0.03 | 0.39 | | NI | 0.02 | NI | 189 | 0.13 | NI | 0.05 |
| | GSE1 | 0.01 | 0.18 | 51 | NI | 0.01 | NI | 50 | 0.05 | NI | 0.02 |
| | HMG20A | 0.01 | 0.18 | 40 | NI | 0.01 | NI | 53 | 0.05 | NI | 0.02 |
| | HMG20B | 0.01 | 0.18 | 50 | NI | 0.01 | NI | 62 | 0.06 | NI | 0.03 |
| | ZMYM2 | 0.02 | 0.26 | NI | NI | 0.01 | NI | 124 | 0.11 | NI | 0.02 |
| | ZMYM3 | | 0.44 | NI | NI | | NI | 255 | | NI | 0.04 |
| NuRD | CHD4 | | 0.63 | 30 | NI | | NI | 170 | | | 0.16 |
| complex | MTA1 | | 0.55 | 15 | NI | 0.02 | NI | 136 | | NI | 0.05 |
| | MTA2 | 0.05 | 0.48 | | NI | 0.02 | NI | | 0.1 | NI | 0.06 |
| | MTA3 | 0.03 | 0.34 | 69 | NI | 0.02 | NI | 87 | 0.1 | NI | 0.05 |
| | MBD2 | NI | | 83 | NI | | NI | 217 | | NI | 0.17 |
| | MBD3 | 0.03 | 0.54 | 59 | NI | 0.02 | NI | 119 | 0.11 | NI | 0.04 |
| | GATAD2A | 0.03 | 0.47 | NI | NI | 0.02 | NI | 183 | 0.12 | NI | 0.06 |
| | GATAD2B | 0.05 | 0.59 | 69 | NI | 0.02 | NI | 106 | 0.1 | NI | 0.07 |
| Sin3 | SIN3A | 0.02 | 0.07 | NI | NI | 0.02 | NI | NI | 0.28 | NI | 0.04 |
| complex | SIN3B | | | NI | NI | 0.03 | NI | NI | 0.43 | NI | 0.04 |
| | SAP30 | | 0.08 | NI | NI | 0.02 | 164 | NI | 0.3 | NI | 0.04 |
| MiDAC | MIDEAS | 0.02 | 0.48 | NI | NI | 0.01 | NI | 246 | 0.18 | NI | 0.04 |
| complex | DNTTIP1 | 0.02 | 0.13 | NI | NI | 0.01 | NI | 271 | 0.15 | NI | 0.03 |
| | CDYL | 0.01 | 0.16 | NI | NI | 0.01 | NI | 257 | 0.1 | NI | 0.03 |
| ELSA | MIER1 | 0.02 | 0.17 | 50 | NI | 0.02 | NI | 73 | 0.18 | NI | 0.06 |
| (ELM-SANT) | MIER2 | | | | | | NI | 99 | 0.11 | NI | |
| domain | MIER3 | 0.01 | 0.16 | 47 | NI | 0.01 | 209 | 34 | 0.07 | NI | 0.02 |
| proteins | RERE | 0.02 | 0.39 | NI | NI | NI | NI | NI | 0.49 | NI | 0.21 |
| | TRERF1 | | | | NI | | | | | | |
| | EHMT2 | 0.02 | | NI | NI | | NI | NI | 0.2 | NI | |
| NCoR | HDAC3 | 0.02 | 0.19 | 7 | 18 | 0.02 | NI | 12 | 0.26 | NI | 0.06 |
| complex | NCOR1 | 0.01 | 0.17 | 7 | 34 | | NI | 21 | 0.19 | NI | 0.04 |
| | NCOR2 | 0.12 | 0.28 | 15 | 26 | 0.04 | NI | 20 | 0.15 | NI | 0.03 |
| | TBL1X | 0.01 | 0.13 | 4 | 20 | 0.02 | NI | 21 | 0.19 | NI | 0.04 |
| | TBL1XR1 | 0.01 | 0.14 | 4 | 20 | 0.01 | NI | 22 | 0.21 | NI | 0.05 |
| | GPS2 | 0.02 | 0.13 | 4 | 37 | 0.02 | 340 | 19 | 0.31 | NI | 0.06 |
| class I HDAC | HDAC8 | NI | 12.38 | NI | NI | NI | NI | NI | NI | 1.85 | NI |
| class II HDAC | HDAC6 | NI | 0.41 | NI | NI | 0.09 | 130 | NI | 1.16 | NI | 0.07 |
| | HDAC10 | NI | 4.43 | NI | NI | 0.07 | 171 | NI | 0.68 | NI | 0.04 |
| selected | BZW2 | NI | 2.25 | NI | NI | 1.71 | 4 | NI | NI | 5.04 | NI |
| HDACi | CBR1 | NI | 1.98 | NI | NI | 0.88 | 19 | 109 | 8.23 | 12.82 | NI |
| off-targets | ALDH1A2 | NI | NI | NI | NI | NI | NI | 230 | NI | NI | NI |
| | ISOC2 | NI | NI | NI | NI | NI | 5 | NI | NI | NI | NI |
| | PPP3CA | NI | 3.5 | NI | NI | 1.51 | 40 | NI | 12.15 | 59.67 | NI |

| | | | | Tacedina- | | | | | | |
|---|---|---|---|---|---|---|---|---|---|---|
| | | | | line | Trichostatin | Valproic | SAHA | | | |
| classification | protein | Romidepsin | Scriptaid | (CI | A (TSA) | acid | (Vorino- | AA-1 | AA-2 | Bufexamac |
| class I HDAC | HDAC1 | 0.01 | 1.03 | 35 | 0.01 | 358 | 0.13 | 134 | NI | NI |
| | HDAC2 | 0.01 | 1.29 | 66 | 0.01 | 471 | 0.12 | 250 | NI | NI |
| CoREST | RCOR1 | 0.01 | 0.74 | 114 | 0.01 | 258 | 0.09 | 408 | NI | NI |
| complex | RCOR2 | 0.01 | 0.69 | 196 | 0.01 | 311 | 0.07 | | NI | NI |
| | RCOR3 | 0.004 | 0.61 | 159 | 0.01 | 274 | 0.07 | NI | NI | NI |
| | PHF21A | 0.02 | 1.09 | | | 438 | | | NI | |
| | GSE1 | 0.01 | 0.73 | 122 | 0.004 | 224 | 0.08 | NI | NI | NI |
| | HMG20A | 0.003 | 0.88 | 139 | | 322 | 0.1 | 258 | NI | NI |
| | HMG20B | 0.004 | 0.77 | 168 | 0.01 | 380 | 0.12 | 293 | NI | NI |
| | ZMYM2 | 0.01 | 0.76 | NI | 0.01 | 700 | 0.11 | NI | NI | 128 |
| | ZMYM3 | | 0.98 | NI | 0.02 | 922 | 0.72 | NI | NI | 172 |
| NuRD | CHD4 | 0.04 | 0.74 | NI | 0.06 | 544 | 0.33 | | NI | 386 |
| complex | MTA1 | | 1.14 | 152 | 0.12 | 528 | 0.17 | NI | NI | 168 |
| | MTA2 | 0.01 | 1.36 | 226 | 0.02 | 997 | 0.15 | NI | NI | 555 |
| | MTA3 | 0.01 | 0.98 | 263 | 0.02 | 667 | 0.15 | 692 | NI | 626 |
| | MBD2 | | 2.41 | NI | | 1302 | | | NI | |
| | MBD3 | 0.01 | 1.2 | 265 | 0.01 | 930 | 0.2 | NI | NI | 409 |
| | GATAD2A | 0.02 | 1.76 | 423 | 0.02 | 1035 | 0.19 | NI | NI | 572 |
| | GATAD2B | 0.01 | 1.46 | 306 | 0.03 | 843 | 0.17 | NI | NI | NI |
| Sin3 | SIN3A | 0.02 | 0.48 | NI | 0.03 | 6485 | 0.07 | NI | NI | 185 |
| complex | SIN3B | | 0.71 | NI | 0.03 | 11999 | 0.11 | NI | NI | NI |
| | SAP30 | | 0.64 | NI | 0.03 | 6651 | 0.09 | NI | NI | 125 |
| MiDAC | MIDEAS | 0.01 | 0.73 | NI | | 611 | 0.07 | NI | NI | 476 |
| complex | DNTTIP1 | 0.01 | 0.66 | NI | 0.02 | 586 | 0.08 | NI | NI | 418 |
| | CDYL | 0.01 | 0.56 | NI | 0.02 | 366 | 0.08 | NI | NI | 151 |
| ELSA | MIER1 | 0.02 | 0.48 | 195 | 0.02 | 507 | 0.11 | | NI | 367 |
| (ELM-SANT) | MIER2 | | 0.81 | 184 | | | | NI | NI | |
| domain | MIER3 | 0.01 | 0.32 | 171 | 0.01 | 344 | 0.05 | 298 | NI | 165 |
| proteins | RERE | 0.09 | 0.79 | NI | 0.05 | 3124 | 0.15 | NI | NI | 33 |
| | TRERF1 | | | | | | *0.24 | | | |
| | EHMT2 | | 1.3 | NI | | 1208 | 0.54 | NI | NI | 105 |
| NCoR | HDAC3 | 0.02 | 3.22 | 18 | 0.03 | 4806 | 0.22 | 31 | 42 | 348 |
| complex | NCOR1 | 0.02 | 1.14 | 60 | 0.02 | 2873 | 0.13 | 37 | 71 | NI |
| | NCOR2 | | 1.83 | 61 | 0.07 | 4326 | 0.09 | 80 | 64 | 418 |
| | TBL1X | | 1.28 | | 0.02 | 2884 | 0.13 | 68 | 75 | 542 |
| | TBL1XR1 | 0.01 | 1.44 | 23 | 0.03 | 1333 | 0.14 | 40 | 57 | 530 |
| | GPS2 | 0.01 | 1.28 | 23 | 0.03 | 2410 | 0.11 | 29 | 43 | 599 |
| class I HDAC | HDAC8 | 0.16 | NI | NI | NI | NI | NI | NI | NI | 243 |
| class II HDAC | HDAC6 | NI | 0.25 | NI | 0.05 | NI | 0.06 | NI | NI | 10 |
| | HDAC10 | NI | NI | NI | NI | NI | 0.78 | NI | NI | 10 |
| selected | BZW2 | NI | 4.84 | NI | NI | NI | 1.97 | NI | NI | 3 |
| HDACi | CBR1 | NI | NI | NI | NI | NI | NI | 540 | NI | 149 |
| off-targets | ALDH1A2 | NI | 5.38 | NI | NI | NI | 0.5 | NI | NI | 3 |
| | ISOC2 | NI | 0.35 | NI | NI | NI | 0.03 | NI | NI | 3 |
| | PPP3CA | NI | NI | NI | NI | NI | NI | NI | NI | 304 |

### REFERENCES OF EXAMPLE 9

1. Kouzarides,T. Chromatin modifications and their function. Cell 128, 693-705 (2007).
2. Choudhary, C. et al. Lysine acetylation targets protein complexes and co-regulates major cellular functions. Science 325, 834-840 (2009).
3. Zhao, S. et al. Regulation of cellular metabolism by protein lysine acetylation. Science 327, 1000-1004 (2010).
4. Karberg, S. Switching on epigenetic therapy. Cell 139, 1029-1031 (2009).
5. Taunton, J., Hassig, C. A., & Schreiber, S. L. A mammalian histone deacetylase related to the yeast transcriptional regulator Rpd3p. Science 272, 408-411 (1996).
6. Gregoretti, I. V., Lee, Y. M., & Goodson, H. V. Molecular evolution of the histone deacetylase family: functional implications of phylogenetic analysis. J. Mol. Biol. 338, 17-31 (2004).
7. Yang, X. J. & Seto, E. The Rpd3/Hda1 family of lysine deacetylases: from bacteria and yeast to mice and men. Nat. Rev. Mol. Cell. Biol. 9, 206-218 (2008).
8. Cunliffe, V. T. Eloquent silence: developmental functions of Class I histone deacetylases. Curr. Opin. Genet. Dev. 18, 404-410 (2008).
9. Lakowski, B., Roelens, I., & Jacob, S. CoREST-like complexes regulate chromatin modification and neuronal gene expression. I Mol. Neurosci. 29, 227-239 (2006).
10. You, A., Tong, J. K., Grozinger, C. M., & Schreiber, S. L. CoREST is an integral component of the CoREST-human histone deacetylase complex. Proc. Natl. Acad. Sci. U. S. A. 98, 1454-1458 (2001).
11. Tong, J. K., Hassig, C. A., Schnitzler, G. R., Kingston, R. E., & Schreiber, S. L. Chromatin deacetylation by an ATP-dependent nucleosome remodelling complex. Nature 395, 917-921 (1998).
12. Zhang, Y., LeRoy, G., Seelig, H. P., Lane, W. S., & Reinberg, D. The dermatomyositis-specific autoantigen Mi2 is a component of a complex containing histone deacetylase and nucleosome remodeling activities. Cell 95, 279-289 (1998).
13. Grzenda, A., Lomberk, G., Zhang, J. S., & Urrutia, R. Sin3: master scaffold and transcriptional corepressor. Biochim. Biophys. Acta 1789, 443-450 (2009).
14. Zhang, Y., Iratni, R., Erdjument-Bromage, H., Tempst, P., & Reinberg, D. Histone deacetylases and SAP18, a novel polypeptide, are components of a human Sin3 complex. Cell 89, 357-364 (1997).
15. Guenther, M. G. et al. A core SMRT corepressor complex containing HDAC3 and TBL1, a WD40-repeat protein linked to deafness. Genes Dev. 14, 1048-1057 (2000).
16. Karagianni,P. & Wong,J. HDAC3: taking the SMRT-N-CoRrect road to repression. Oncogene 26, 5439-5449 (2007).
17. Bradner,J. E. et al. Chemical phylogenetics of histone deacetylases. Nat. Chem. Biol. 6, 238-243 (2010).
18. Lahm,A. et al. Unraveling the hidden catalytic activity of vertebrate class IIa histone deacetylases. Proc. Natl. Acad. Sci. U S. A 104, 17335-17340 (2007).
19. Boyault, C., Sadoul,K., Pabion,M., & Khochbin,S. HDAC6, at the crossroads between cytoskeleton and cell signaling by acetylation and ubiquitination. Oncogene 26, 5468-5476 (2007).
20. Marks,P.A. & Breslow,R. Dimethyl sulfoxide to vorinostat: development of this histone deacetylase inhibitor as an anticancer drug. Nat. Biotechnol. 25, 84-90 (2007).
21. Bolden,J.E., Peart,M.J., & Johnstone,R.W. Anticancer activities of histone deacetylase inhibitors. Nat. Rev. Drug Discov. 5, 769-784 (2006).
22. Zhang,Y. et al. Analysis of the NuRD subunits reveals a histone deacetylase core complex and a connection with DNA methylation. Genes Dev. 13, 1924-1935 (1999).
23. Guenther,M.G., Barak,O., & Lazar,M.A. The SMRT and N-CoR corepressors are activating cofactors for histone deacetylase 3. Mol. Cell. Biol. 21, 6091-6101 (2001).
24. Salisbury, C.M. & Cravatt,B.F. Activity-based probes for proteomic profiling of histone deacetylase complexes. Proc. Natl. Acad. Sci. U S. A 104, 1171-1176 (2007).
25. Bantscheff,M. et al. Quantitative chemical proteomics reveals mechanisms of action of clinical ABL kinase inhibitors. Nat. Biotechnol. 25, 1035-1044 (2007).
26. Ong,S. E. et al. Identifying the proteins to which small-molecule probes and drugs bind in cells. Proc. Natl. Acad. Sci. U. S. A 106, 4617-4622 (2009).
27. Sharma,K. et al. Proteomics strategy for quantitative protein interaction profiling in cell extracts. Nat. Methods 6, 741-744 (2009).
28. Cravatt,B.F., Wright,A.T., & Kozarich,J.W. Activity-based protein profiling: from enzyme chemistry to proteomic chemistry. Annu. Rev. Biochem. 77, 383-414 (2008).
29. Bantscheff,M., Scholten,A., & Heck,A.J. Revealing promiscuous drug-target interactions by chemical proteomics. Drug Discov. Today 14, 1021-1029 (2009).
30. Marks,P.A. & Dokmanovic,M. Histone deacetylase inhibitors: discovery and development as anticancer agents. Expert. Opin. Investig. Drugs 14, 1497-1511 (2005).
31. Bantscheff,M. et al. Robust and sensitive iTRAQ quantification on an LTQ Orbitrap mass spectrometer. Mol. Cell. Proteomics. 7, 1702-1713 (2008).
32. Kruhlak,M. J. et al. Regulation of global acetylation in mitosis through loss of histone acetyltransferases and deacetylases from chromatin. J. Biol. Chem. 276, 38307-38319 (2001).
33. Savitski,M. M. et al. Targeted Data Acquisition for Improved Reproducibility and Robustness of Proteomic Mass Spectrometry Assays. J. Am. Soc. Mass Spectrom. (2010).
34. Wang,Y. et al. LSD1 is a subunit of the NuRD complex and targets the metastasis programs in breast cancer. Cell 138, 660-672 (2009).
35. Wang,L., Charroux,B., Kerridge,S., & Tsai, C.C. Atrophin recruits HDAC1/2 and G9a to modify histone H3K9 and to determine cell fates. EMBO Rep. 9, 555-562 (2008).
36. Trommer,H. et al. Examinations of the antioxidative properties of the topically administered drug bufexamac reveal new insights into its mechanism of action. J. Pharm. Pharmacol. 55, 1379-1388 (2003).
37. Alberts,B. The cell as a collection of protein machines: preparing the next generation of molecular biologists. Cell 92, 291-294 (1998).
38. Gavin,A. C. et al. Proteome survey reveals modularity of the yeast cell machinery. Nature 440, 631-636 (2006).
39. Scheiber,J. et al. Gaining insight into off-target mediated effects of drug candidates with a comprehensive systems chemical biology analysis. J. Chem. Inf. Model. 49, 308-317 (2009).
40. Malovannaya,A. et al. Streamlined analysis schema for high-throughput identification of endogenous protein complexes. Proc. Natl. Acad. Sci. U S. A 107, 2431-2436 (2010).
41. Beckers,T. et al. Distinct pharmacological properties of second generation HDAC inhibitors with the benzamide or hydroxamate head group. Int. J. Cancer 121, 1138-1148 (2007).
42. Blackwell,L., Norris,J., Suto, C.M., & Janzen,W.P. The use of diversity profiling to characterize chemical modulators of the histone deacetylases. Life Sci. 82, 1050-1058 (2008).
43. Khan,N. et al. Determination of the class and isoform selectivity of small-molecule histone deacetylase inhibitors. Biochem. J. 409, 581-589 (2008).
44. Smith,K.T., Martin-Brown,S.A., Florens,L., Washburn,M.P., & Workman,J.L. Deacetylase inhibitors dissociate the histone-targeting ING2 subunit from the Sin3 complex. Chem. Biol. 17, 65-74 (2010).
45. Kubota,T., Maezawa,S., Koiwai,K., Hayano,T., & Koiwai, O. Identification of functional domains in TdIF1 and its inhibitory mechanism for TdT activity. Genes Cells 12, 941-959 (2007).
46. Mulligan,P. et al. CDYL bridges REST and histone methyltransferases for gene repression and suppression of cellular transformation. Mol. Cell. 32, 718-726 (2008).
47. Balasubramanian,S. et al. A novel histone deacetylase 8 (HDAC8)-specific inhibitor PCI-34051 induces apoptosis in T-cell lymphomas. Leukemia 22, 1026-1034 (2008).
48. Wang,Z. et al. Genome-wide mapping of HATs and HDACs reveals distinct functions in active and inactive genes. Cell 138, 1019-1031 (2009).

### Methods

### 1. Reagents.

All reagents were purchased from Sigma unless otherwise noted below. Antibodies were purchased from the following suppliers: sc-7872 (HDAC1), sc-81599 (HDAC2), sc-17795 (HDAC3), sc-11405 (HDAC8), sc-81325 (MTA3), sc-100908 (TBL1XR1), sc-81082 and sc-166296 (DNTTIP1) and sc47778 (□-actin) from Santa Cruz; 05-814 (HDAC2), 05-813 (HDAC3), 07-505 (HDAC8) from Millipore; ab46985 (HDAC1), ab3479 (Sin3A), ab70039 (DNTTIP1); NB100-81655 (TRERF1) and NB100-81654 (TRERF1) from Novus Biologicals; and ab61236 (H4-AcK5) from Abcam; H-3034 (HDAC-3) and T-6793 (Ac-tubulin) from Sigma; H00010013-M01 (HDAC6) from Abnova; #2184 (LSD1) from Cell Signaling Technologies; and 382158 (H3-AcK9/K14) from Calbiochem. Secondary antibodies labelled with IRDye®680 and IRDye®800 were from LICOR, and antibodies labelled with Alexa®480 and Alexa®594 were from Invitrogen. Reference compounds were purchased from the following suppliers: vorinostat (SAHA), belinostat (PXD-101), dacinostat (LAQ-824), panobinostat (LBH-589), PCI-24781, and entinostat (MS-275) from Selleck; trichostatin A, PCI-34051, bufexamac and apicidin from Sigma; scriptaid and tacedinaline (CI-994) from Tocris; MC-1293 and BML-210 from Enzo Life Sciences; MGCD-0103 from Chemietek; romidepsin (FK-228) from ACC Corp; valproic acid from Calbiochem, and SRT1720 from Cayman.

### 2. Cell Culture and Cell-Based Assays.

Jurkat E6.1, HL60, Ramos and HeLa cells were purchased from ATCC; K562 cells were purchased from DSMZ (Braunschweig, Germany). Jurkat E6.1 cells were cultured in RPMI1640 supplemented with 4.5 g/L glucose, 10 mM Hepes, 1 mM sodium pyruvate and 10% Fetal Calf Serum (FCS). Ramos cells were cultured in RPMI1640 containing 10% FCS. K562 cells were cultured in RPMI medium containing 10% FCS. Cells were expanded to maximal 1×10⁶ cells/ml. HeLa cells were cultured in Minimum Essential Media (MEM) supplemented with 1 mM pyruvate, 0.1 mM nonessential amino acids and 10% FCS. For indirect immunofluorescence experiments, the FCS content was reduced to 2%. For cell cycle arrest of HeLa cells in G1/S-phase or mitosis cells were treated for 16 hours with 15 μg/ml aphidicolin (Sigma) or with 0.3 μM nocodazole (Sigma). Control HeLa cells were treated with DMSO for 16 hours. For cell-based protein acetylation assays, HeLa or K562 cells (96-well format, 5×10⁴ cells per well) were treated with compounds for 6 hours. Cells were washed with cold PBS and lysed directly in SDS-sample buffer, followed by denaturation at 95° C. Lysates (10 μl) were resolved on SDS-gels, transferred to PVDF membranes and analyzed for tubulin and histone acetylation by immune-detection using IRDye⁸-labelled secondary antibodies and a LiCOR Odyssey scanner. Data analysis of the quantified bands was performed using Excel and GraphPad Prism. For the IFNα secretion assay, human peripheral blood mononuclear cells were isolated with Histopaque 1077 (Sigma) from fresh human donor blood and were plated at a concentration of 0.5×10⁶ cells/ml. Cells were incubated with test compounds for 45 minutes prior to stimulation with plasmacytoid dendritic cell-specific TLR9 agonist ODN2216 (Invivogen). Interferon (IFN)-α released into cell supernatants was measured in triplicates after 16 hours by Flex-Set IFNalpha (BD Biosciences) by flow cytometry (FACSCalibur, BD Biosciences). For all cell-based assays viability was assessed in parallel (MTT kit, Roche).

### 3. Indirect Immunofluorescence Analysis.

HeLa cells were plated to sub-confluency on polylysine-coated glass chamber slides and treated after recovery with the indicated compounds for 4 hours. Samples were fixed in cold methanol, permeabilized with Triton-X100, blocked in 1% BSA and treated for immuno-detection of acetylated tubulin and acetylated histone H3. Cells were counterstained for nucleic acids using 4′,6-diamidino-2-phenylindole (DAPI). Multichannel fluorescence microscopy was performed on an Olympus IX-70 microscope. Images were acquired using a monochrome CCD camera (CoolSNAP HQ Digital) and analyzed with MetaMorph (Universal Imaging Corporation). The instrument was adjusted to ensure proper comparison of levels of acetylated tubulin and acetylated histone H3 before and after inhibitor treatment.

### 4. Preparation of Cell Lysates.

Frozen cell pellets were homogenized in lysis buffer (50 mM Tris-HCl, 0.8% Igepal-CA630, 5% glycerol, 150 mM NaCl, 1.5 mM MgCl2, 25 mM NaF, 1 mM sodium vanadate, 1 mM DTT, pH 7.5). One complete EDTA-free protease inhibitor tablet (Roche) per 25 ml was added. The sample was dispersed using a Dounce homogenizer, kept rotating for 30 minutes at 4° C. and spun for 10 minutes at 20,000×g at 4° C. The supernatant was spun again for 1 hour at 145,000×g. The protein concentration was determined by Bradford assay (BioRad), and aliquots were snap frozen in liquid nitrogen and stored at −80° C.

### 5. Compound Profiling.

Affinity profiling assays were performed as described previously^{1,2} with minor modifications. Derivatized sepharose beads (35 μl beads per sample) were equilibrated in lysis buffer and incubated with 1 ml (5 mg protein) cell lysate, which had been preincubated with test compound or vehicle, on an end-over-end shaker for 1 hour at 4° C. Beads were transferred to disposable columns (MoBiTec), washed with lysis buffer containing 0.2% detergent and eluted with 50 μl 2×SDS sample buffer. Proteins were alkylated with 200 mg/ml iodoacetamide for 30 minutes, separated on 4-12% NuPAGE (Invitrogen), and stained with colloidal Coomassie.

### 6. Compound Screening.

The compounds for screening was selected either for their potential to be Zinc chelators or based on a similarity search of the compound collections from Asinex Corp. (Winston-Salem, N.C., USA) and Enamine (Mammouth_Jct., NJ, USA), using a training set of 140 known HDAC inhibitors. Competition binding assays using SAHA-beads were performed essentially as described above but adapted to a 96-well format. 1 mg of cell lysate and 5 μl of beads were used per well. Compounds from the screening library including reference compounds as standards were added at 20 μM and 100 μM final concentration from 50×DMSO stocks. Each plate contained 8 positive (TSA 50 μM) and eight negative controls (2% DMSO). Beads were eluted in SDS sample buffer (100 mM Tris pH 7.4, 4% SDS, 20% glycerol, 0.01% bromophenol blue, 50 mM DTT) and spotted in duplicate on nitrocellulose membranes (600 nl/spot) using an automated liquid dispenser (FluidX). After drying, the membranes were rehydrated in 20% ethanol, and processed for detection with specific antibodies as indicated. Spot intensities were quantified using a LiCOR Odyssey scanner and percentage inhibition was calculated using positive and negative controls as 100% and 0% inhibition respectively.

### 7. Quantitative Co-Immunopurification.

Antibodies were tested for suitability in co-IP assays by standard immunoprecipitation (IP)-western procedures³. Western blotting was performed using a LI-COR Odyssey System. Suitable antibodies (40-100 μg) were coupled to 100 μl AminoLink resin (Thermo Fisher Scientific). Cell lysate samples (10 mg) were incubated with pre-washed immuno resin on a shaker for 2 hours at 4° C. Beads were washed in lysis buffer containing 0.4% Igepal-CA630 and lysis buffer without detergent. Bound proteins were eluted in 100 μl 2×SDS sample buffer. Protein samples were reduced, alkylated and separated by SD-PAGE. To provide a specificity control for quantitative LC-MS analysis, IgG from the same species was used in an analogous “mock IP” carried out in parallel from an aliquot of the same lysate sample. Typically, four IP reactions, which were subsequently combined in a single iTRAQ sample for MS/MS analysis, were performed in parallel, two with different antibodies directed against the same (or different) antigen(s) and two “mock IP” samples.

### 8. Mass Spectrometry Sample Preparation.

Gels were cut into slices across the entire separation range and subjected to in-gel digestion. Peptide extracts were labeled with iTRAQ™ reagents (Applied Biosystems) or TMT™ (Thermo-Fisher Scientific) in 40 mM triethylammoniumbicarbonate (TEAB), pH 8.53. After quenching of the reaction with glycin labeled extracts were combined. For compound profiling experiments extracts from vehicle treated samples were labeled with TMT reagent 131, and combined with extracts from compound-treated samples labeled with TMT reagents 126-131, fractionated using reversed phase chromatography at pH 12, dried and acidified prior to LC-MS/MS analysis.

### 9. LC-MS/MS Analysis.

Samples were dried in vacuo and re-suspended in 0.1% formic acid in water and aliquots of the sample were injected into a nano-LC system (Eksigent 1D+) coupled to LTQ-Orbitrap mass spectrometers (Thermo-Finnigan). Peptides were separated on custom 50 cm×75 uM (ID) reversed phase columns (Reprosil) at 40° C. Gradient elution was performed from 2% acetonitrile to 40% acetonitrile in 0.1% formic acid over 2-3 hrs. LTQ-Orbitrap XL and Orbitrap Velos instruments were operated with XCalibur 2.0/2.1 software. Intact peptides were detected in the Orbitrap at 30.000 resolution. Internal calibration was performed using the ion signal from (Si(CH₃)₂O)₆H⁺ at m/z 445.120025⁴. Data dependent tandem mass spectra were generated for up to six peptide precursors using a combined CID/HCD approach⁵ or using HCD at a resolution of 7500 for histone modification data. For CID up to 5000 ions (Orbitrap XL) or up to 3000 ions (Orbitrap Velos) were accumulated in the ion trap within a maximum ion accumulation time of 200 msec. For HCD target ion settings were 50000 (Orbitrap XL) and 25000 (Orbitrap Velos), respectively.

### 10. Peptide and Protein Identification.

Mascot™ 2.0 (Matrix Science) was used for protein identification using 10 ppm mass tolerance for peptide precursors and 0.8 Da (CID) or 20 mDa (HCD) tolerance for fragment ions. Carbamidomethylation of cysteine residues and iTRAQ/TMT modification of lysine residues were set as fixed modifications and S,T,Y phosphorylation, methionine oxidation, N-terminal acetylation of proteins and iTRAQ/TMT modification of peptide N-termini were set as variable modifications. The search data base consisted of a customized version of the IPI protein sequence database combined with a decoy version of this database created using a script supplied by Matrix Science⁶. Unless stated otherwise, we accepted protein identifications as follows: i) For single spectrum to sequence assignments, we required this assignment to be the best match and a minimum Mascot score of 31 and a 10× difference of this assignment over the next best assignment. Based on these criteria, the decoy search results indicated <1% false positive identification rate; ii) For multiple spectrum to sequence assignments and using the same parameters, the decoy search results indicate <0.1% false positive identification rate. For protein quantification a minimum of 2 sequence assignments matching to unique peptides was required. False positive identification rate for quantified proteins was <<0.1%. Localization of post-translational modifications on histone peptides was validated by remapping the de-isotoped and de-convoluted tandem MS spectra to b and y ions expected from the peptide hit within 20 ppm mass accuracy. Only SSMs where fragment ions support localization on only one amino acid were considered for further analysis.

### 11. Peptide and Protein Quantification.

Centroided iTRAQ/TMT reporter ion signals were computed by the XCalibur software operating and extracted from MS data files using customized scripts. Only peptides unique for identified proteins were used for relative protein quantification. Reporter ion intensities were multiplied with the ion accumulation time yielding an area value proportional to the number of reporter ions present in the mass analyzer. For compound competition binding experiments fold changes are reported based on reporter ion areas in comparison to vehicle control and were calculated using a linear model. Fold changes were corrected for isotope purity as described and adjusted for interference caused by co-eluting nearly isobaric peaks as estimated by the s2i measure⁷. Dose-response curves were fitted using R software⁸ and the drc software⁹ as described previously¹. IC₅₀ values were confirmed in replicate experiments using targeted data acquisition⁷ for a subset of proteins. In order to compare selectivities of compounds displaying different absolute potencies relative potencies were calculated as (pIC₅₀−min(pIC₅₀))/(max(pIC₅₀)−min(pIC₅₀)) for each experiment. For the robustness estimation (FIG. 32b), displayed pIC₅₀ values were median normalized. Apparent dissociation constants (K_{D}^{app}) were derived from IC₅₀ values as described¹⁰. For immunoprecipitations, the Enrichment E was calculated as (A(IP)−A(mock IP))/(A(IP)+A(mock IP)) and scales between 0 and 1. ‘A’ represents the summed-up reporter ion response for the protein of interest. Relative enrichment (FIG. 33a) was calculated as RE(IP1)═((A(IP1)−A(mock IP))/(A(IP1)+A(IP2)+A(mock IP)). A RE of 0.5 means that the protein was precipitated in both IPs equally well.

### 12. Heat Map Generation.

Heat maps and t-tests were performed using the R-software package. For unbiased hierarchical clustering of compound profiling data, all quantified proteins identified in at least 10 independent experiments were considered. Protein and compound clustering was based on relative potency using the Euclidean distance measure and the complete linkage method provided in R.

### 13. Synthetic Procedures

13.1 Synthesis of a Methylamino SAHA Analogue N1-(4-(aminomethyl)phenyl)-N-8-hydroxyoctanediamide for Immobilization to Beads

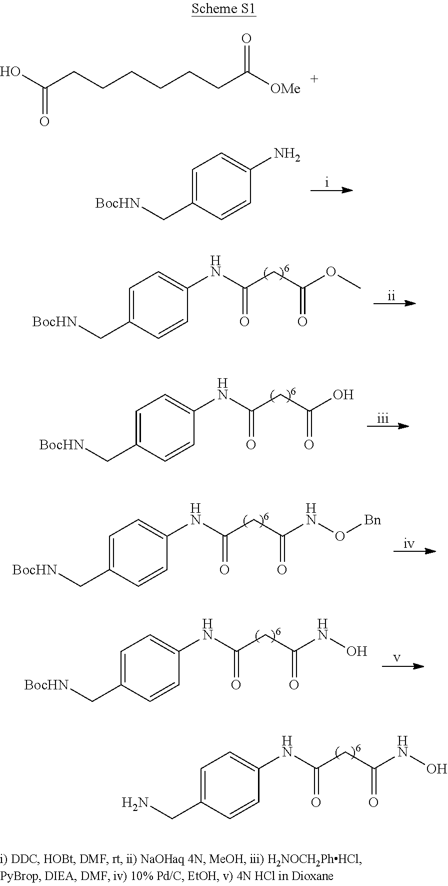

13.2 Synthesis of methyl 8-(4-((tert-butoxycarbonylamino)methyl)phenylamino)-8-oxooctanoate.

To a stirred solution of suberic acid monomethyl ester (3.75 mmol, 1 eq, 0.706 g), 4[N-Boc aminomethyl]aniline (4.5 mmol, 1.2 eq, 1 g), Hydroxybenzotrizol (4.5 mmol, 1.2 eq, 0.608 g) in DMF (20 ml), dicyclocarbodiamide (4.5 mmol, 1.2 eq, 0.928 g) was added. The reaction was stirred at room temperature overnight. The reaction precipitate was filtered. The filtrate was treated with water (20 ml). The resulting precipitate was filtered. The filtrate was then concentrated and purified by Flash Chromatography (hexane/ethyl acetate 1:1) to yield the desired compound as a white solid (m=1.25 g, 88%). LC/MS analysis yielded Rt=2.93 nm, M+H=393, M+Na=415.

13.3 Synthesis of 8-(4-((tert-butoxycarbonylamino)methyl)phenylamino)-8-oxooctanoic acid.

To a stirred solution of 8-(4-((tert-butoxycarbonylamino)methyl)phenylamino)-8-oxooctanoate (0.579 mmol, 0.227 g) in methanol (7 ml) was added 2N aqueous sodium hydroxide (1.16 mmol, 2 eq, 0.580 ml). The reaction was stirred at room temperature overnight and the solvent was removed. The residue was dissolved in water. The pH was raised to 6 by addition of 2N aqueous hydrochloric acid. The resulting precipitate was filtered and dried in a vacuum oven (40° C.) overnight to yield the desired compound as a white solid (m=0.169 g, 77%) LC/MS analysis yielded Rt=2.48 nm M+H=379, M+Na=401.

13.4 Synthesis of tert-butyl 4-(8-(benzylaminooxy)-8-oxooctanamido)benzylcarbamate.

To a stirred solution of 8-(4-((tert-butoxycarbonylamino)methyl)phenylamino)-8-oxooctanoic acid (0.397 mmo, 1 eq, 0.150 g) and O-benzylhydroxylamine hydrochloride (0.397 mmol, 1 eq, 0.063 g) in dimethylformamide (5 ml) and diisopropylethylamine (1.59 mmol, 4 eq, 0.277 ml) was added bromo-tris-pyrrolidino phosphoniumhexafluorophosphate (0.595 mmol, 1.5 eq, 0.277 g). The reaction was stirred at room temperature overnight, diluted with water (10 ml) and extracted with ethyl acetate (2×50 ml). The organic layers were dried over magnesium sulphate, then concentrated and purified by flash chromatography (hexane/ethyl acetate (30-100%) to yield the desired product as an oil (m=0.105 g, 55%) LC/MS analysis yielded rt=2.73 nm, M+H=484, M+Na=506.

13.5 Synthesis of tert-butyl 4-(8-(aminooxy)-8-oxooctanamido)benzylcarbamate.

To a degassed solution of tert-butyl 4-(8-(benzylaminooxy)-8-oxooctanamido)benzylcarbamate (0.787 mmol, 1 eq, 0.380 g) in ethanol (15 ml) was added 10% Pd/C (10%, 38 mg). The reaction was saturated in hydrogen and stirred under hydrogen atmosphere overnight. The catalyst was filtered and the filtrate concentrated to yield the desired compound as a yellow oil (0.309 g, 99%). LC/MS analysis yielded RT=2.18 nm, M+H=394, M+Na=416.

13.6 Synthesis of N-(4-(aminomethyl)phenyl)-8-(aminooxy)-8-oxooctanamide.

To tert-butyl 4-(8-(aminooxy)-8-oxooctanamido)benzylcarbamate (0.22 mmol, 0.150 g) was added 2 ml of hydrochloric acid 4N in dioxane. The mixture was stirred at room temperature 3 hours. The reaction was evaporated and purified by HPLC (high pH) to yield the desired compound as an off white solid (m=51 mg, 46%). LC/MS analysis yielded Rt=1.46 nm 2M+H=587, M+Na=316. ¹NMR (DMSO-d6, 400 MHz): δ=9.81 (s, 1H), 7.51 (d, 2H), 7.23 (d, 2H), 3.64 (s, 2H), 2.27 (d, 2H), 1.93 (d, 2H), 1.56 (m, 2H), 1.48 (m, 2H), 1.26 (m, 4H).

### 13.7 Preparation of the HDAC Affinity Matrix.

Beads were prepared by immobilizing N1-(4-(aminomethyl)phenyl)-N-8-hydroxyoctanediamide (analogue of SAHA) and (6-(aminomethyl)naphthalen-2-yl)methyl (4-(hydroxycarbamoyl)phenyl)carbamate (analogue of ITF2357), on NHS-activated Sepharose 4 beads (GE Healthcare), as described previously^{1,2}. Ligand concentrations in the coupling reaction were 2 and 3 mmol/mL beads for the SAHA and ITF2357 analogs, respectively, and the completion of the coupling reaction was monitored by HPLC. Beads were washed with 10 ml of DMSO and isopropanol and were stored in isopropanol at −20° C.

13.8 Synthesis of a Methylamino ITF2357 Analogue (6-(aminomethyl)naphthalen-2-yl)methyl (4-(hydroxycarbamoyl)phenyl)carbamate for Immobilization to Beads.

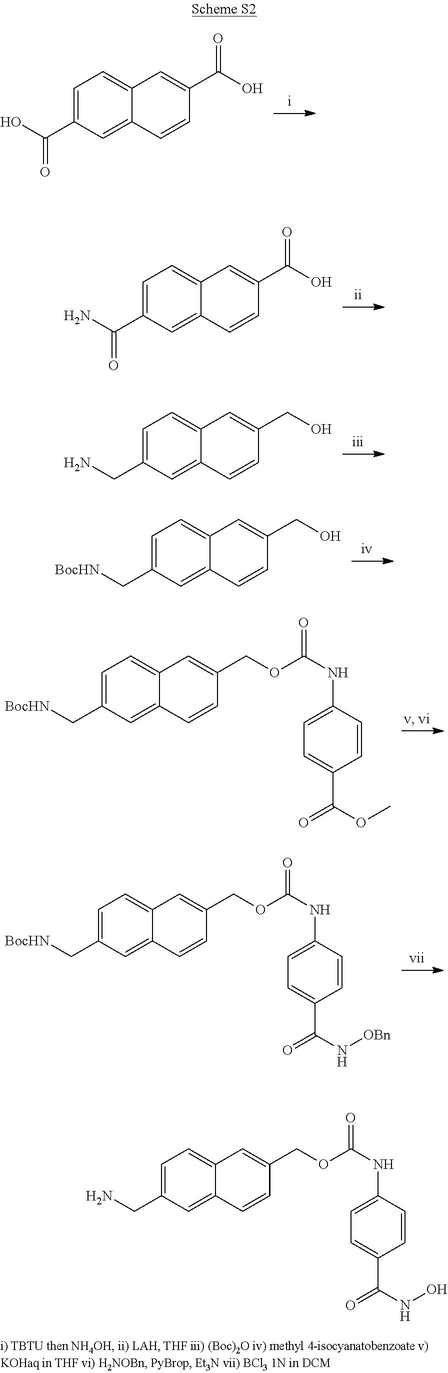

13.9 Synthesis of 6-carbamoyl-2-naphthoic acid.

To a solution of naphthalene-2,6-dicarboxylic acid (55.6 mmol, 12 g) in DMF (11) under nitrogen N-methyl morpholine (83.3 mmol, 93.1 ml) was added followed by TBTU (55.6 mmol, 17.8 g). The mixture was stirred at room temperature for 2 hours. Ammonium hydroxide (85% weight, 166.7 mmol, 9 ml) was then added and the reaction stirred at room temperaturet overnight. The reaction was filtered, the filtrate partitioned between saturated NaHCO₃ and Ethyl acetate (11). The aqueous layer was separated and acidified with 5N HCl. The resulting solid was separated, washed with water then with Ethyl acetate. The solid was dried to yield the desired compound (10.9 g) as a mixture of desired product and starting material (2:1) which was carried to the next step without further purification. LCMS Rt=2.73 nm M+H=216.

13.10 Synthesis of (6-(aminomethyl)naphthalen-2-yl)methanol.

To a stirred suspension of LiAlH₄ (203 mmol, 7.7 g) in THF (300 ml) was added slowly (over 1 hour) 6-carbamoyl-2-naphthoic acid (mixture 2:1 of desired product/di acid from the previous step). The reaction was stirred for 6 days. LiAlH₄ (50.7 mmol, 2 g) was added and the reaction refluxed overnight. The reaction was poured on ice/water. The solid was filtered and the organic layer separated. The organic layer was treated with 1N HCl, the solid removed, the aqueous layer basified, the solid removed and the filtrate extracted with DCM to yield the desired compound as a white solid (1.2 g). ¹NMR (DMSO-d6, 400 MHz): δ=7.9-7.7 (m, 4H), 7.6-7.5 (q, 2H), 4.6 (s, 2H), 3.85 (s, 2H).

13.11 Synthesis of tert-butyl ((6-(hydroxymethyl)naphthalen-2-yl) methyl)carbamate.

(Boc)₂O (7.1 mmol, 1.54 g) in DCM (10 ml) was added slowly to a solution of (6-(aminomethyl)naphthalen-2-yl)methanol (6.4 mmol, 1.2 g) in DCM (200 ml) at 0° C. The reaction was then stirred at room temperature overnight, then was washed with water (50 ml), and the organic layer dried over Na₂SO₄. Purification by flash chromatography (10 to 50% EtOAc in Pet Ether) yielded the desired compound as a white solid (0.53 mg, 29% yield). LCMS Rt=3.77 nm M+H=288.

13.12 Synthesis of methyl 4-((((6-(((tert-butoxycarbonyl)amino)methyl)naphthalen-2-yl)methoxy)carbonyl)amino)benzoate.

To a solution of tert-butyl ((6-(hydroxymethyl)naphthalen-2-yl)methyl)carbamate (1.25 mmol, 0.360 g) in toluene (50 ml) under nitrogen was added slowly methyl 4-isocyanatobenzoate (3.14 mmol, 0.55 mg). The reaction was stirred at 50° C. overnight, then cooled at room temperature. The resulting precipitate was filtered, dissolved in DCM and extracted 5 times with water. The organic layer was dried over MgSO4, and evaporated to yield the desired compound as a white solid (0.420 g, 72% yield). LCMS: Rt=4.47 nm M−H=463.

13.13 Synthesis of tert-butyl ((6-((((4-((benzyloxy)carbamoyl)phenyl)carbamoyl)oxy)-methyl)naphthalen-2-yl)methyl)-carbamate.

To a solution of methyl 4-((((6-(((tert-butoxycarbonyl)amino)methyl)naphthalen-2-yl)methoxy)carbonyl)amino)benzoate (0.58 mmol, 0.27 g) in THF (20 ml) a solution of 0.1M KOH in water (1.16 mmol, 11.6 ml) was added. The reaction was stirred at 50° C. overnight, then evaporated to give a white solid. The solid was dissolved in DMF (70 ml) followed by triethylamine (5.4 mmol, 0.75 ml), O-benzylhydroxylamine (1.16 mmol, 0.185 g), and PyBrop (1.16 mmol, 0.600 g). The reaction was stirred at room temperature overnight. The solvent was removed and the residue triturated in DCM. The solid was filtered to yield the desired compound as a white solid (0.180 g, 54% yield). LCMS: Rt=4.29 nm M−H=554.

13.14 Synthesis of (6-(aminomethyl)naphthalen-2-yl)methyl (4-(hydroxycarbamoyl)phenyl)-carbamate.

BCl₃ 1N in DCM (1.8 mmol, 1.8 ml) was added to a solution of tert-butyl ((6-((((4-((benzyloxy)carbamoyl)phenyl)carbamoyl)oxy)methyl)naphthalen-2-yl)methyl)carbamate (0.18 mmol, 0.100 g) in THF (25 ml) at 0° C. The reaction was then refluxed for 1 h. After cooling on ice, 1N HCl (20 ml) was added. The reaction was then evaporated and loaded on a SCX column in methanol. The column was then eluted with 5%, then 10%, then 20% ammonia in methanol. The combined eluates were evaporated to yield the desired compound as a white solid (0.35 mg, 53% yield). LCMS (method C): Rt=2.44 nm M−H=364. ¹NMR (DMSO-d6, 400 MHz): δ=10 (s, 1H), 7.9 (m, 4H), 7.7 (m, 2H), 7.55 (0, 4H), 5.3 (s. 2H), 3.9 (s, 2H).

### REFERENCES OF THE METHOD SECTION OF EXAMPLE 9

1. Bantscheff,M. et al. Quantitative chemical proteomics reveals mechanisms of action of clinical ABL kinase inhibitors. Nat. Biotechnol. 25, 1035-1044 (2007)
2. Drewes,G. et al. Process for the identification of novel enzyme interacting compounds. Patent WO 2006/134056 A1.
3. Harlow,E. & Lane,D. Antibodies: a Laboratory Manual. 1988. New York, Cold Spring Harbor Laboratory Publications.
4. Olsen,J. V. et al. Parts per million mass accuracy on an Orbitrap mass spectrometer via lock mass injection into a C-trap. Mol. Cell. Proteomics. 4, 2010-2021 (2005).
5. Köcher,T. et al. High precision quantitative proteomics using iTRAQ on an LTQ Orbitrap: a new mass spectrometric method combining the benefits of all. J. Proteome. Res. 8, 4743-4752 (2009).
6. Elias,J.E., Haas,W., Faherty,B.K., & Gygi,S.P. Comparative evaluation of mass spectrometry platforms used in large-scale proteomics investigations. Nat. Methods 2, 667-675 (2005).
7. Savitski,M. M. et al. Targeted Data Acquisition for Improved Reproducibility and Robustness of Proteomic Mass Spectrometry Assays. J. Am. Soc. Mass Spectrom.(2010).
8. R Development Core Team R: A language and environment for statistical computing. Vienna, Austria, 2007).
9. Ritz, C. & Streibig,J.C. Bioassay Analysis using R. J. Statist. Software 12, (2007).
10. Sharma,K. et al. Proteomics strategy for quantitative protein interaction profiling in cell extracts. Nat. Methods 6, 741-744 (2009).

1. A method for the identification of a histone deacetylase (HDAC) interacting compound, comprising the steps of
a) providing an HDAC protein complex containing protein preparation derived from a cell endogenously expressing an HDAC,
b) contacting the protein preparation with a non-proteinaceous HDAC ligand immobilized on a solid support and with a given compound thereby allowing the reversible binding of said ligand to said HDAC protein complex, and
c) determining to what extent in step b) a binding between the ligand and the protein complex has occurred.

2. A method for the identification of a histone deacetylase (HDAC) interacting compound, comprising the steps of
a) providing two aliquots of an HDAC protein complex containing protein preparation derived from a cell endogenously expressing an HDAC,
b) contacting one aliquot with a non-proteinaceous HDAC ligand immobilized on a solid support thereby allowing the reversible binding of said ligand to said HDAC protein complex,
c) contacting the other aliquot with a non-proteinaceous HDAC ligand immobilized on a solid support and with a given compound thereby allowing the reversible binding of said ligand to said HDAC protein complex, and
d) determining to what extent in steps b) and c) a binding between the ligand and the protein complex has occurred.

3. A method for the identification of a histone deacetylase (HDAC) interacting compound, comprising the steps of:
a) providing two aliquots of a cell preparation each comprising at least one cell endogenously expressing an HDAC,
b) incubating one aliquot with a given compound,
c) harvesting the cells of each aliquot,
d) lysing the cells of each aliquot in order to obtain protein preparations containing an HDAC protein complex,
e) contacting the protein preparations with a non proteinaceous HDAC ligand immobilized on a solid support thereby allowing the reversible binding of said ligand to said HDAC protein complex, and
f) determining to what extent in step e) a binding between the ligand and the protein complex has occurred.

4. The method of any of claims 1 to 3, wherein the HDAC is selected from the group consisting of HDAC1, HDAC2, HDAC3, HDAC6, HDAC8, and HDAC10.

5. The method of any of claims 2 to 4, wherein a reduced binding observed for the aliquot incubated with the compound in comparison to the aliquot not incubated with the compound indicates that HDAC is a target of the compound.

6. The method of any of claims 1 to 5, wherein the extent of the binding is determined by separating the HDAC protein complex from the ligand and subsequent detection of a component of the separated HDAC protein complex or subsequent determination of the amount of a component of the separated HDAC protein complex.

7. The method of claim 6, wherein said detection or said determination is performed by mass spectrometry or immunodetection methods, preferably with an antibody directed against the component.

8. The method of any of claims 1 to 7, wherein said given compound is selected from the group consisting of synthetic compounds, or organic synthetic drugs, more preferably small molecule organic drugs, and natural small molecule compounds.

9. The method of any of claims 1 to 8, wherein the given compound is an HDAC inhibitor.

10. The method of any of claims 1 to 9, wherein the provision of a protein preparation includes the steps of harvesting at least one cell endogenously expressing an HDAC and lysing the cell.

11. The method of any of claims 1 to 10, wherein the binding of the ligand to the HDAC protein complex steps is performed under essentially physiological conditions.

12. The method of any of claims 1 to 11, performed as an high-throughput-method.

13. A method for the identification of a histone deacetylase (HDAC) interacting compound, comprising the steps of
a) providing at least two aliquots of an HDAC containing protein preparation derived from a cell endogenously expressing an HDAC,
b) contacting one aliquot with a non-proteinaceous HDAC ligand immobilized on a solid support thereby allowing the reversible binding of said ligand to HDAC,
c) contacting the remaining aliquots with an HDAC ligand immobilized on a solid support and with various concentrations of a compound thereby allowing the reversible binding of said ligand to HDAC,
d) separating the HDAC from the ligand, and
e) determining by mass spectrometry or immunodetection the amount of HDAC eluted from the ligand, thereby determining to what extent in steps b) and c) a binding between the ligand and the protein has occurred.

14. The method of claim 13, with the features as defined in any of claims 4, 5, and 8 to 11.

15. The method of any of claims 1 to 14, wherein the ligand has a molecular weight of less than 1000 Da.
